(19) United States
(12) Patent Application Publication
(10) Pub. No.: US 2007/0185055 A1
(43) Pub. Date: Aug. 9, 2007

(54) Method for treating cachexia with retinoid ligands

(76) Inventors: Guang Liang Jiang, Lake Forest, CA (US); Yang-Dar Yuan, Irvine, CA (US); Roshantha A. Chandraratna, Laguna Hills, CA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/348,520

(22) Filed: Feb. 6, 2006

(51) Int. Cl. A61K 31/695; A61K 31/382; A61K 31/353; A61K 31/192; A61K 31/215

(52) U.S. Cl. 514/63; 514/569; 514/531; 514/456; 514/432

(57) ABSTRACT

The present invention relates to a method of treatment of cachexia in a subject in need of treatment. More specifically, the present invention relates to the use of retinoid compounds that act on retinoid X receptors (RXRs) for the treatment of cachexia in a subject in need of treatment. The cachexia is associated with, in other words a complication of, a primary disease, condition or disorder. Primary diseases, conditions and disorders include, but are not limited to, cancer, AIDS, liver cirrhosis, diabetes mellitus, chronic renal failure, chronic obstructive pulmonary disease, chronic cardiac failure, immune system diseases (e.g., rheumatoid arthritis and systemic lupus erythematosus), tuberculosis, cystic fibrosis, gastrointestinal disorders (e.g., irritable bowel syndrome and inflammatory bowel disease), Parkinson's disease, anorexia nervosa, dementia, major depression, an aged condition and sarcopenia.

### RELATED APPLICATIONS

This application is a continuation of International Application No. PCT/2004/025564, which designated the United States and was filed on Aug. 6, 2004, published in English, which claims the benefit of U.S. Provisional Application No. 60/493,138, filed on Aug. 7, 2003 and U.S. Provisional Application No. 60/533,734, filed on Dec. 31, 2003. The entire teachings of the above applications are incorporated herein by reference.

### BACKGROUND OF THE INVENTION

Cachexia, which literally means ‘bad condition’, refers to involuntary weight loss, anorexia (loss of appetite), loss of protein and fat mass, gain in the proportion of body-water, and a variety of metabolic changes, which are associated with a primary disease, condition or disorder. Diseases, conditions or disorders which are typically associated with cachexia include, but are not limited to, cancer, AIDS, liver cirrhosis, diabetes mellitus, chronic renal failure, chronic obstructive pulmonary disease, chronic cardiac failure, immune system diseases (e.g., rheumatoid arthritis and systemic lupus erythematosus), tuberculosis, cystic fibrosis, gastrointestinal disorders (e.g., irritable bowel syndrome and inflammatory bowel disease), Parkinson's disease, dementia, anorexia nervosa, major depression, an aged condition and sarcopenia. Cachexia is a strong independent risk factor for morbidity and mortality. Cancer cachexia occurs in about half of all cancer patients.

The fact that a large proportion of cancer patients have cachexia, coupled with the demonstrated relationship between cachexia and mortality has provided impetus for the search into underlying mechanisms and therapies that might prevent or reverse cachexia. However, this need has gone largely unmet.

### SUMMARY OF THE INVENTION

The present invention relates to a method of treating of cachexia in a subject in need of treatment. More specifically, the present invention relates to the use of retinoid compounds that act on retinoid X receptors (RXRs) for the treating of cachexia in a subject in need of treatment. The cachexia is associated with, in other words a complication of, a primary disease, condition or disorder. Primary diseases, conditions and disorders include, but are not limited to, cancer, AIDS, liver cirrhosis, diabetes mellitus, chronic renal failure, chronic obstructive pulmonary disease, chronic cardiac failure, immune system diseases (e.g., rheumatoid arthritis and systemic lupus erythematosus), tuberculosis, cystic fibrosis, gastrointestinal disorders (e.g., irritable bowel syndrome and inflammatory bowel disease), Parkinson's disease, dementia, major depression, anorexia nervosa, an aged condition and sarcopenia. In one embodiment, the cachexia is associated with one or more of AIDS, liver cirrhosis, diabetes mellitus, chronic renal failure, chronic obstructive pulmonary disease, chronic cardiac failure, immune system diseases, tuberculosis, cystic fibrosis, gastrointestinal disorders, an aged condition and sarcopenia. In another embodiment, the cachexia is associated with one or more of cancer, AIDS, liver cirrhosis, chronic renal failure, chronic obstructive pulmonary disease, chronic cardiac failure, immune system diseases, tuberculosis, cystic fibrosis, gastrointestinal disorders, an aged condition and sarcopenia. In yet another embodiment, the cachexia is associated with one or more of AIDS, liver cirrhosis, chronic renal failure, chronic obstructive pulmonary disease, chronic cardiac failure, immune system diseases, tuberculosis, cystic fibrosis, gastrointestinal disorders, an aged condition and sarcopenia. In a specific embodiment, the cachexia is associated with cancer. In another specific embodiment, the cachexia is associated with AIDS.

In one embodiment, the method of treating cachexia in a subject in need thereof comprises administering to the subject a therapeutically effect amount of a compound represented by Structural Formula (I):

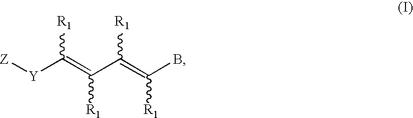

where:

Z is represented by Structural Formula (II) or Structural Formula (III)

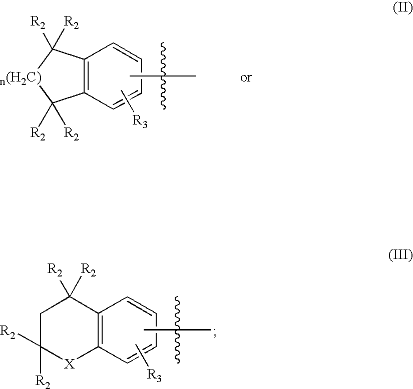

Y is cycloalkyl of 3 to 8 carbons or cycloalkenyl of 5 to 8 carbons optionally substituted with one or two R₄ groups, or Y is selected from phenyl, pyridyl, thienyl, furyl, pyrrolyl, pyridazinyl, pyrimidinyl, pyrazinyl, thiazolyl, oxazolyl, and imidazolyl, said groups being optionally substituted with one or two R₄ groups, and wherein Y is substituted by the Z and —CR₁═CR₁—CR₁═CR₁— groups on adjacent carbons;

X is S, O, or NR₅;

n is 1 or 2;

R₁ and R₂ independently are —H, lower alkyl or fluoroalkyl;

R₃ is hydrogen, lower alkyl, alkylamino, dialkylamino, cyano, —Cl or —Br;

R₄ is lower alkyl, fluoroalkyl or halogen;

R₅ is H or lower alkyl;

B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl;

R₇ is an alkyl, cycloalkyl or alkenyl group containing 1 to 5 carbons;

R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl;

R₉ and R₁₀ independently are hydrogen, an alkyl group of 1 to 10 carbons, or a cycloalkyl group of 5 to 10 carbons, or phenyl or lower alkylphenyl;

R₁₁ is lower alkyl, phenyl or lower alkylphenyl;

R₁₂ is lower alkyl; and

R₁₃ is divalent alkyl radical of 2 to 5 carbons.

In a particular embodiment, Z is represented by Structural Formula (II) or (III); Y is selected from pyridyl, pyrrolyl, pyridazinyl, pyrimidinyl, pyrazinyl, thiazolyl, oxazolyl, and imidazolyl, said groups being optionally substituted with one or two R₄ groups, and wherein Y is substituted by the Z and —CR₁═CR₁—CR₁═CR₁— groups on adjacent carbons; X is NR₅; n is 1 or 2; R₁ and R₂ independently are —H, lower alkyl or fluoroalkyl; R₃ is hydrogen, lower alkyl, —Cl or —Br; R₄ is lower alkyl, fluoroalkyl or halogen; R₅ is —H or lower alkyl; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR,₂)₂, —CHOR₁₃O, —COR₇, —CR₇(OR₁₂)₂, —CR₇OR₁₃O, or tri(lower alkyl)silyl; R₇ is an alkyl, cycloalkyl or alkenyl group containing 1 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is a divalent alkyl radical of 2 to 5 carbons.

In another particular embodiment, Z is represented by Structural Formula (III); Y is thienyl or furyl, said thienyl or furyl groups being optionally substituted with one or two R₄ groups, and wherein Y is substituted by the Z and —CR₁═CR₁—CR₁═CR₁— groups on adjacent carbons; X is NR₅; n is 1 or 2; R₁ and R₂ independently are —H, lower alkyl or fluoroalkyl; R₃ is hydrogen, lower alkyl, —Cl or —Br; R₄ is lower alkyl, fluoroalkyl or halogen; R₅ is H or lower alkyl; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃, —COR₇, —CR₇(OR₁₂)₂, —CR₇OR₁₃O, or tri(lower alkyl)silyl; R₇ is an alkyl, cycloalkyl or alkenyl group containing 1 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is a divalent alkyl radical of 2 to 5 carbons.

In yet another particular embodiment, Z is represented by Strucutural Formula (III); Y is cycloalkyl of 3 to 8 carbons or cycloalkenyl of 5 to 8 carbons optionally substituted with one or two R₄ groups, or Y is selected from phenyl, pyridyl, thienyl, furyl, pyrrolyl, pyridazinyl, pyrimidinyl, pyrazinyl, thiazolyl, oxazolyl, and imidazolyl, said groups being optionally substituted with one or two R₄ groups, and wherein Y is substituted by the Z and —CR₁═CR₁—CR₁═CR₁— groups on adjacent carbons; X is S or O; n is 1 or 2; R₁ and R₂ independently are H, lower alkyl or fluoroalkyl; R₃ is hydrogen, lower alkyl, Cl or Br; R₄ is lower alkyl, fluoroalkyl or halogen; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl; R₇ is an alkyl, cycloalkyl or alkenyl group containing 1 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is divalent alkyl radical of 2 to 5 carbons.

In a further particular embodiment of compounds represented by Structural Formula (I), Z is represented by Structural Formula (II); Y is selected from thienyl or furyl, said groups being optionally substituted with one or two R₄ groups, and wherein Y is substituted by the Z and —CR₁═CR₁—CR₁═CR₁— groups on adjacent carbons; n is 1 or 2; R₁ and R₂ independently are H, lower alkyl or fluoroalkyl; R₃ is hydrogen, lower alkyl, alkylamino, dialkylamino, cyano, Cl or Br; R₄ is lower alkyl, fluoroalkyl or halogen; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl; R₇ is an alkyl, cycloalkyl or alkenyl group containing 1 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is divalent alkyl radical of 2 to 5 carbons.

Another group of compounds encompassed by Structural Formula (I) include those where Z is represented by Structural Formula (III); Y is cycloalkyl of 3 to 8 carbons or cycloalkenyl of 5 to 8 carbons optionally substituted with one or two R₄ groups, or Y is phenyl, said groups being optionally substituted with one or two R₄ groups, and wherein Y is substituted by the Z and —CR₁═CR₁—CR₁═CR₁— groups on adjacent carbons; X is NR₅; R₁ and R₂ independently are H, lower alkyl or fluoroalkyl; R₃ is hydrogen, lower alkyl, alkylamino, dialkylamino, cyano, Cl or Br; R₄ is lower alkyl, fluoroalkyl or halogen; R₅ is —H or lower alkyl; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl; R₇ is an alkyl, cycloalkyl or alkenyl group containing 1 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is divalent alkyl radical of 2 to 5 carbons.

Yet another group of compounds encompassed by Structural Formula (I) include those where Z is represented by Structural Formula (III); Y is cyclopropyl, said Y group being optionally substituted with one or two R₄ groups, and wherein Y is substituted by the Z and —CR₁═CR₁—CR1═CR₁— groups on adjacent carbons; X is NR₅; R₁ and R₂ independently are H, lower alkyl or fluoroalkyl; R₃ is hydrogen, lower alkyl, alkylamino, dialkylamino, cyano, Cl or Br; R₄ is lower alkyl, fluoroalkyl or halogen; R₅ is —H or lower alkyl; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl; R₇ is an alkyl of 1 to 5 carbons, cycloalkyl of 3 to 5 carbons or alkenyl group containing 2 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is divalent alkyl radical of 2 to 5 carbons.

In another embodiment, the invention includes a method of treating cachexia in a subject in need of treatment comprising administering a therapeutically effect amount of a compound represented by Structural Formula (IV):

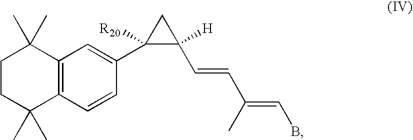

where R₂₀ is alkyl of 1 to 6 carbons, and B is —COOH, or —COOR₂, where R₂₁ is alkyl of 1 to 6 carbons, or a pharmaceutically acceptable salt of said compound.

Another aspect of the invention is where a therapeutically effect amount of a compound represented by Structural Formula (V) is used in a method of treating cachexia in a subject in need of treatment therefor:

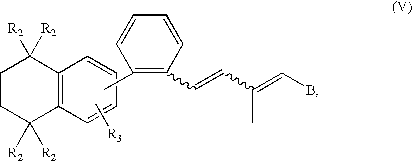

where:

R₂ is hydrogen or lower alkyl;

R₃ is hydrogen or lower alkyl;

B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri-lower alkylsilyl;

R₇ is an alkyl, cycloalkyl or alkenyl group containing 1 to 5 carbons;

R₈ is an alkyl group of 1 to 10 carbons, a cycloalkyl group of 5 to 10 carbons or trimethylsilylalkyl where the alkyl group has 1 to 10 carbons, or R₈ is phenyl or lower alkylphenyl;

R₉ and R₁₀ independently are hydrogen, an alkyl group of 1 to 10 carbons, or a cycloalkyl group of 5 to 10 carbons, or phenyl or lower alkylphenyl;

R₁₁ is lower alkyl, phenyl or lower alkylphenyl;

R₁₂ is lower alkyl; and

R₁₃ is divalent alkyl radical of 2 to 5 carbons.

The invention further includes a method of treating a subject in need thereof for cachexia, comprising administering a therapeutically effective amount of a compound represented by Structural Formula (VI):

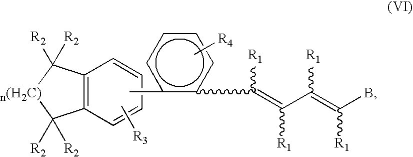

where:

n is 1 or 2;

R₁ and R₂ independently are —H, lower alkyl or fluoroalkyl;

R₃ is hydrogen, lower alkyl, —Cl or —Br;

R₄ is H, lower alkyl, fluoroalkyl or halogen;

B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri-lower alkylsilyl;

R₇ is an alkyl, cycloalkyl or alkenyl group containing 1 to 5 carbons;

R₈ is an alkyl group of 1 to 10 carbons, a cycloalkyl group of 5 to 10 carbons or trimethylsilylalkyl where the alkyl group has 1 to 10 carbons, or R₈ is phenyl or lower alkylphenyl;

R₉ and R₁₀ independently are hydrogen, an alkyl group of 1 to 10 carbons, or a cycloalkyl group of 5 to 10 carbons, or phenyl or lower alkylphenyl;

R₁₁ is lower alkyl, phenyl or lower alkylphenyl;

R₁₂ is lower alkyl; and

R₁₃ is divalent alkyl radical of 2 to 5 carbons.

In another embodiment, the method of treating cachexia in a subject in need thereof includes administering a therapeutically effective amount of a compound represented by Structural Formula (VII):

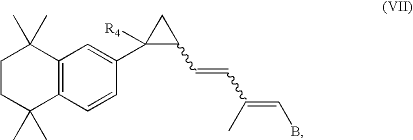

where:

R₄ is lower alkyl of 1 to 6 carbons;

B is —COOH or —COOR₈; and

R₈ is lower alkyl of 1 to 6 carbons; and

the configuration about the cyclopropane ring is cis, and the configuration about the double bonds in the pentadienoic acid or ester chain attached to the cyclopropane ring is trans in each of said double bonds, or a pharmaceutically acceptable salt thereof.

In yet another embodiment, the compounds administered for treating cachexia in a subject in need thereof are represented by Structural Formula (VIII):

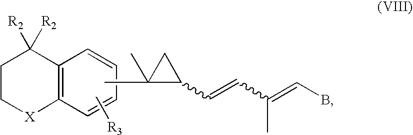

wherein:

X is S or O; alternatively, X is NR₅;

R₂ is hydrogen or lower alkyl;

R₃ is hydrogen or lower alkyl;

R₅ is hydrogen or lower alkyl;

B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri-lower alkylsilyl;

R₇ is an alkyl, cycloalkyl or alkenyl group containing 1 to 5 carbons, such as an alkyl of 1 to 5 carbons, a cycloalkyl of 3 to 5 carbons or an alkenyl group containing 2 to 5 carbons;

R₈ is an alkyl group of 1 to 10 carbons, a cycloalkyl group of 5 to 10 carbons or trimethylsilylalkyl where the alkyl group has 1 to 10 carbons, or R₈ is phenyl or lower alkylphenyl;

R₉ and R₁₀ independently are hydrogen, an alkyl group of 1 to 10 carbons, or a cycloalkyl group of 5 to 10 carbons, or phenyl or lower alkylphenyl;

R₁₁ is lower alkyl, phenyl or lower alkylphenyl;

R₁₂ is lower alkyl; and

R₁₃ is divalent alkyl radical of 2 to 5 carbons.

In a preferred embodiment, compounds of Structural Formula (I) for treating cachexia are represented by Structural Formulas (IX), (X) and (XI):

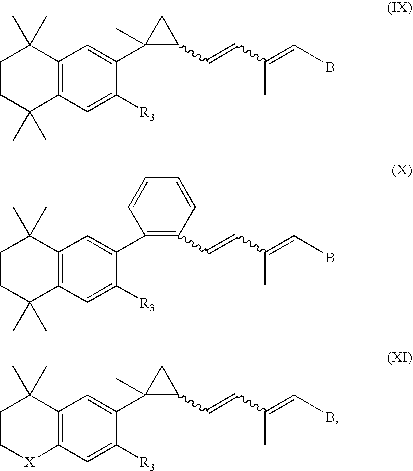

where:

B is —COOH or —COOR₈;

R₃ is hydrogen, lower alkyl, —Cl or —Br;

R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; and

X is S or O.

Another aspect of the invention involves treating cachexia in a subject in need thereof comprising administering an effective amount of a compound represented by any one of Structural Formulas (XIII), (XIV) or (XV):

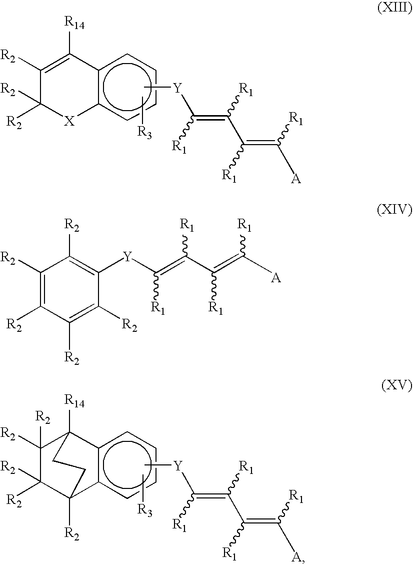

where:

X is O, S, or (CR₁R₁)ₙ;

n is 0, 1 or 2;

Y is a bivalent radical having Structural Formula (XVI) or Structural Formula (XVII) where p is an integer from 1 to 4:

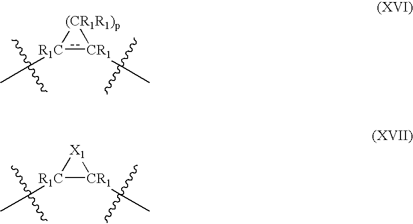

or Y is a bivalent aryl or 5 or 6 membered heteroaryl radical having 1 to 3 heteroatoms selected from N, S and O, said aryl or heteroaryl groups being unsubstituted, or substituted with 1 to 3 C₁₋₆ alkyl or with 1 to 3 C₁₋₆ fluoroalkyl groups;

X is O, S or NH;

R₁ is independently —H, lower alkyl of 1 to 6 carbons, or lower fluoroalkyl of 1 to 6 carbons;

R₂ is independently —H, lower alkyl of 1 to 6 carbons, —OR₁, 1-adamantyl, or lower fluoroalkyl of 1 to 6 carbons, or the two R₂ groups jointly represent an oxo group;

R₃ is hydrogen, lower alkyl of 1 to 6 carbons, —OR₁, fluoro substituted lower alkyl of 1 to 6 carbons or halogen, —NO₂, —NH₂, —NHCO(C₁-C₆)alkyl, or —NHCO(C₁-C₆)alkenyl;

A is hydrogen, COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CH(OR₁₃O), —COR₇, —CR₇(OR₁₂)₂, —CR₇(OR₁₃O), or Si(C₁₋₆ alkyl)₃;

R₇ is an alkyl, cycloalkyl or alkenyl group containing 1 to 5 carbons;

R₈ is an alkyl group of 1 to 10 carbons or (trimethylsilyl)alkyl where the alkyl group has 1 to 10 carbons, or a cycloalkyl group of 5 to 10 carbons, or R₈ is phenyl or lower alkylphenyl;

R₉ and R₁₀ independently are hydrogen, an alkyl group of 1 to 10 carbons, or a cycloalkyl group of 5-10 carbons, or phenyl, hydroxyphenyl or lower alkylphenyl;

R₁₁ is lower alkyl, phenyl or lower alkylphenyl;

R₁₂ is lower alkyl;

R₁₃ is divalent alkyl radical of 2-5 carbons; and

R₁₄ is alkyl of 1 to 10 carbons, fluoro-substituted alkyl of 1 to 10 carbons, alkenyl of 2 to 10 carbons and having 1 to 3 double bonds, alkynyl having 2 to 10 carbons and 1 to 3 triple bonds, carbocyclic aryl selected from the group consisting of phenyl, C₁-C₁₀-alkylphenyl, naphthyl, C₁-C₁₀-alkylnaphthyl, phenyl-C₁-C₁₀ alkyl, naphthyl-C₁-C₁₀ alkyl, C₁-C₁₀-alkenylphenyl having 1 to 3 double bonds, C₁-C₁₀-alkynylphenyl having 1 to 3 triple bonds, phenyl-C₁-C₁₀ alkenyl having 1 to 3 double bonds, phenyl-C₁-C₁₀ alkynyl having 1 to 3 triple bonds, hydroxy alkyl of 1 to 10 carbons, hydroxyalkenyl having 2 to 10 carbons and 1 to 3 double bonds, hydroxyalkynyl having 2 to 10 carbons and 1 to 3 triple bonds, acyloxyalkyl of 1 to 10 carbons, acyloxyalkenyl having 2 to 10 carbons and 1 to 3 double bonds, or acyloxyalkynyl of 2 to 10 carbons and 1 to 3 triple bonds where the acyl group is represented by COR₈, or R₁₄ is a 5 or 6 membered heteroaryl group having 1 to 3 heteroatoms, said heteroatoms being selected from a group consisting of O, S, and N, said heteroaryl group being unsubstituted or substituted with a C₁ to C₁₀ alkyl group, with a C₁ to C₁₀ fluoroalkyl group, or with halogen, and the dashed line in Structural Formula (XVI) represents a bond or absence of a bond.

A further aspect of the invention is a method of treating cachexia in a subject in need thereof comprising administering a therapeutically effective amount of a compound represented by Structural Formula (XVIII):

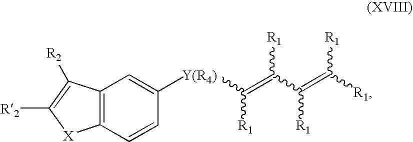

wherein:

X is O, NR′ or S;

R′ is alkyl of 1 to 6 carbons;

Y is a bivalent cyclopropyl radical optionally substituted with one or two R₄ groups, or Y is a bivalent aryl or 5 or 6 membered heteroaryl radical having 1 to 3 heteroatoms selected from N, S and O, said aryl or heteroaryl groups optionally substituted with 1 to 4 R₄ groups;

R₁ is independently —H, alkyl of 1 to 6 carbons, or fluoroalkyl of 1 to 6 carbons;

R₂ is alkyl of 1 to 8 carbons, or fluoroalkyl of 1 to 8 carbons;

R′₂ is alkyl of 1 to 8 carbons, or fluoroalkyl of 1 to 8 carbons;

R₃ is hydrogen, alkyl of 1 to 6 carbons, fluoro substituted alkyl of 1 to 6 carbons, halogen, alkoxy of 1 to 8 carbons, or alkylthio of 1 to 6 carbons, —NO₂, —NH₂, —NHCO(C₁-C₆)alkyl, —NHCO(C₁-C₆)alkenyl, —NR₁H or —N(R₁)₂, benzyloxy or C₁-C₆ alkyl-substituted benzyloxy;

R₄ is —H or alkyl of 1 to 6 carbons, or fluoro substituted alkyl of 1 to 6 carbons;

m is an integer having the values of 0 to 3, and

B is —COOH or a pharmaceutically acceptable salt thereof, —COOR₈, —COOCH₂COR₇, —CONR₉R₁₀, —CH₂OH, —CH₂OR₁₁, —CH₂OCOR₁₁, —CHO, —CH(OR₁₂)₂, —CH(OR₁₃O), —COR₇, —CR₇(OR₁₂)₂, —CR₇(OR₁₃O),

R₇ is an alkyl, cycloalkyl or alkenyl group containing 1 to 5 carbons;

R₈ is an alkyl group of 1 to 10 carbons or (trimethylsilyl)alkyl where the alkyl group has 1 to 10 carbons, or a group of 5 to 10 phenyl or lower alkylphenyl;

R₉ and R₁₀ independently are hydrogen, an alkyl group of 1 to 10 carbons, or a cycloalkyl group of 5-10 carbons, or phenyl, hydroxyphenyl or lower alkylphenyl;

R₁₁ is lower alkyl, phenyl or lower alkylphenyl;

R₁₂ is lower alkyl; and

R₁₃ is divalent alkyl radical of 2-5 carbons.

Yet another aspect of the invention is a method of treating cachexia in a subject in need thereof with a therapeutically effective amount of a compound represented by Structural Formula (XIX):

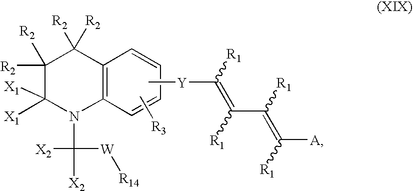

wherein:

Y is a bivalent radical having Formula (a) or Formula (b):

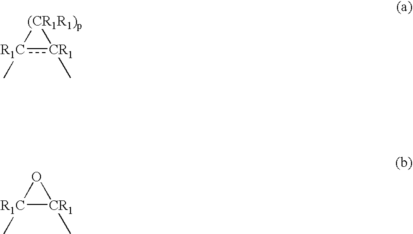

or Y is a bivalent aryl or 5 or 6 membered heteroaryl radical having 1 to 3 heteroatoms selected from N, S and O, said aryl or heteroaryl groups being unsubstituted, or substituted with 1 to 3 C₁₋₆ alkyl or with 1 to 3 C₁₋₆ fluoroalkyl groups;

p is an integer from 1 to 4;

the two X₁ groups jointly represent an oxo or thione function, or X₁ is independently selected from —H or alkyl of 1 to 6 carbons;

the two X₂ groups jointly represent an oxo or a thione function, or X₂ is independently selected from —H or alkyl of 1 to 6 carbons, with the proviso that one of the joint X₁ grouping or of the joint X₂ grouping represents an oxo or a thione function;

W is —H, —O—, —C(R₁)₂—, phenyl, naphthyl, or 5 or 6 membered heteroaryl group having 1 to 3 heteroatoms, said heteroatoms being selected from a group consisting of O, S, and N, said phenyl, naphthyl or heteroaryl groups being unsubstituted or substituted with a C₁ to C₁₀ alkyl group, with a C₁ to C₁₀ fluoroalkyl group, or with halogen;

R₁ is independently —H, lower alkyl of 1 to 6 carbons, or lower fluoroalkyl of 1 to 6 carbons;

R₂ is independently —H, lower alkyl of 1 to 6 carbons, or lower fluoroalkyl of 1 to 6 carbons;

R₃ is hydrogen, lower alkyl of 1 to 6 carbons, —OR₁, fluoro substituted lower alkyl of 1 to 6 carbons or halogen, —NO₂, —NH₂, —NHCO(C₁-C₆ alkyl, or —NHCO(C₁-C₆)alkenyl;

A is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CH(OR₁₃O), —COR₇, —CR₇(OR₁₂)₂, —CR₇(OR₁₃O), or —Si(C₁₋₆ alkyl)₃;

R₇ is an alkyl, cycloalkyl or alkenyl group containing 1 to 5 carbons,

R₈ is an alkyl group of 1 to 10 carbons or (trimethylsilyl)alkyl where the alkyl group has 1 to 10 carbons, or a cycloalkyl group of 5 to 10 carbons, or R₈ is phenyl or lower alkylphenyl;

R₉ and R₁₀ independently are hydrogen, an alkyl group of 1 to 10 carbons, or a cycloalkyl group of 5-10 carbons, or phenyl, hydroxyphenyl or lower alkylphenyl;

R₁₁ is lower alkyl, phenyl or lower alkylphenyl;

R₁₂ is lower alkyl;

R₁₃ is divalent alkyl radical of 2-5 carbons;

R₁₄ is —H, alkyl of 1 to 10 carbons, fluoro-substituted alkyl of 1 to 10 carbons, alkenyl of 2 to 10 carbons and having 1 to 3 double bonds, alkynyl having 2 to 10 carbons and 1 to 3 triple bonds, carbocyclic aryl selected from the group consisting of phenyl, C₁-C₁₀-alkylphenyl, naphthyl, C₁-C₁₀-alkylnaphthyl, phenyl-C₁-C₁₀ alkyl, naphthyl-C₁-C₁₀-alkyl, C₁-C₁₀-alkenylphenyl having 1 to 3 double bonds, C₁-C₁₀-alkynylphenyl having 1 to 3 triple bonds, phenyl-C₁-C₁₀ alkenyl having 1 to 3 double bonds, phenyl-C₁-C₁₀ alkynyl having 1 to 3 triple bonds, hydroxy alkyl of 1 to 10 carbons, hydroxyalkenyl having 2 to 10 carbons and 1 to 3 double bonds, hydroxyalkynyl having 2 to 10 carbons and 1 to 3 triple bonds, acyloxyalkyl of 1 to 10 carbons, acyloxyalkenyl having 2 to 10 carbons and 1 to 3 double bonds, or acyloxyalkynyl of 2 to 10 carbons and 1 to 3 triple bonds where the acyl group is represented by COR₈, or R₁₄ is a 5 or 6 membered heteroaryl group having 1 to 3 heteroatoms, said heteroatoms being selected from a group consisting of O, S, and N, said carbocyclic aryl and heteroaryl groups being unsubstituted or substituted with a C₁ to C₁₀ alkyl group, with a C₁ to C₁₀ fluoroalkyl group, or with halogen;

and the dashed line in Formula (a) represents a bond or absence of a bond, provided that when the dashed line represents a bond then there are no R₁ substituents on the carbons connected by said bond.

In another embodiment, the invention is a method of treating cachexia in a subject in need thereof comprising administering a therapeutically effective amount of a compound represented by Structural Formula (XX):

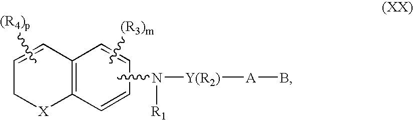

wherein:

X is O, S, or C(R)₂;

R is —H or alkyl of 1 to 6 carbons;

R₁ is —H, alkyl of 1 to 10 carbons, alkenyl of 2 to 6 carbons, phenyl-C₁-C₆ alkyl, or C₁-C₆-alkylphenyl;

R₂ is H, alkyl of 1 to 6 carbons, —F, —Cl, —Br, —I, —CF₃, fluoro substituted alkyl of 1 to 6 carbons, alkoxy of 1 to 6 carbons, or alkylthio of 1 to 6 carbons;

R₃ is independently alkyl of 1 to 6 carbons, —F, —Cl, —Br, —I, —CF₃, fluoro substituted alkyl of 1 to 6 carbons, —OH, —SH, alkoxy of 1 to 6 carbons, fluoroalkoxy of 1 to 6 carbons, alkylthio of 1 to 6 carbons, benxyloxy, C₁-C₆ alkyl substituted benzyloxy, halogen substituted benzyloxy, phenyloxy, C₁-C₆ alkyl substituted phenyloxy, or halogen substituted phenyloxy;

R₄ is independently —H, alkyl of 1 to 6 carbons, or —F;

Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups; m is an integer having the values 0 to 3;

p is an integer having the values 0 to 4;

A is —(CH₂)_{q}— where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;

B is hydrogen, —COOH, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri-lower alkylsilyl;

R₇ is an alkyl, cycloalkyl or alkenyl group containing 1 to 5 carbons,

R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl;

R₉ and R₁₀ independently are hydrogen, an alkyl group of 1 to 10 carbons, or a cycloalkyl group of 5-10 carbons, or phenyl or lower alkylphenyl;

R₁₁ is lower alkyl, phenyl or lower alkylphenyl;

R₁₂ is lower alkyl; and

R₁₃ is divalent alkyl radical of 2-5 carbons, and pharmaceutically acceptable salts thereof.

In a further embodiment, the invention is a method of treating cachexia in a subject in need thereof comprising administering a therapeutically effective amount of a compound represented by any one of Structural Formula (XXI), (XXII), (XXIII), (XXIV), (XXV), (XXXVI), (XXXVII), (XXVIIa) or (XXVIIb):

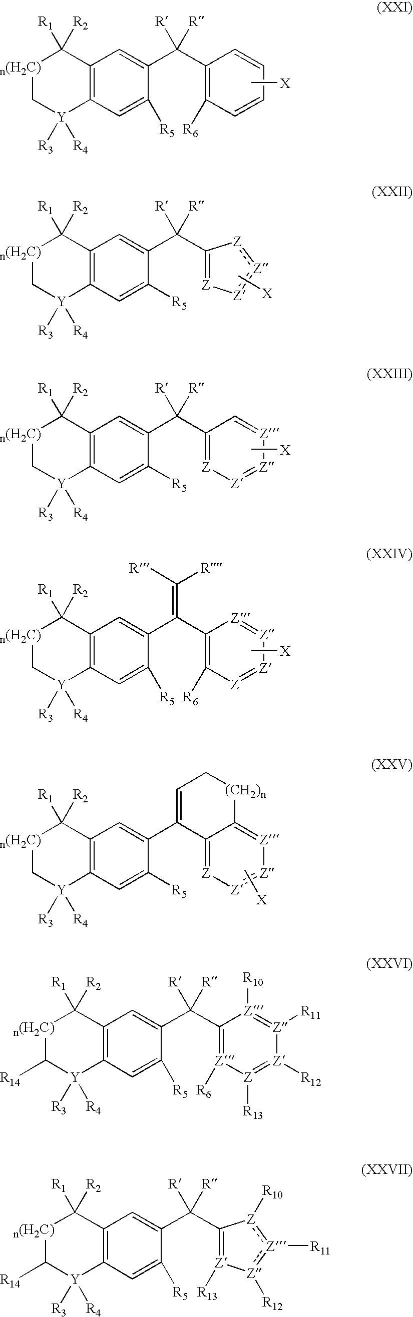

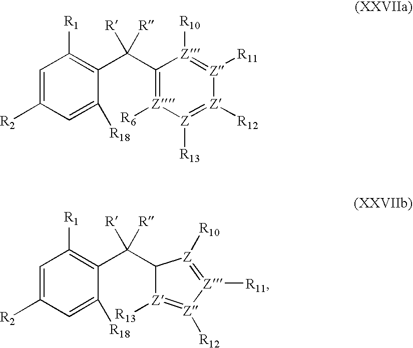

wherein:

R₁ and R₂ each independently is hydrogen or lower alkyl or acyl having 1-4 carbon atoms;

Y is C, O, S, N, CHOH, CO, SO, SO₂, or a pharmaceutically acceptable salt;

R₃ is hydrogen or lower alkyl having 1-4 carbon atoms where Y is C or N;

R₄ is hydrogen or lower alkyl having 1-4 carbon atoms when Y is C, R₄ does not exist if Y is N, or neither R₃ or R₄ exist if Y is S, O, CHOH, CO, SO, or SO₂;

R′ and R″ are hydrogen, lower alkyl or acyl having 1-4 carbon atoms, —OH, alkoxy having 1-4 carbon atoms, thiol or thioether, or amino, or R′ or R″ taken together form an oxo(keto), methano, thioketo, HO—N═, NC—N═, (R₇R₈)N—N═, R₁₇O—N═, R₁₇N═, epoxy, cyclopropyl, or cycloalkyl group and wherein the epoxy, cyclopropyl, and cycloalkyl groups are optionally substituted with lower alkyl having 1-4 carbons or halogen;

R′″ and R″″ are hydrogen, halogen, lower alkyl or acyl having 1-4 carbon atoms, alkylamino, or R′″ and R″″ taken together form a cycloalkyl group having 3-10 carbons, and wherein the cycloalkyl group can be substituted with lower alkyl having 1-4 carbons or halogen;

R₅ is hydrogen, a lower alkyl having 1-4 carbons, halogen, nitro, —OR₇, —SR₇, —NR₇R₈, or —(CF)ₙCF₃, but R₅ is not hydrogen if R₆, R₁₀, R₁₁, R₁₂ and R₁₃ are all hydrogen, Z, Z′, Z″, Z′″, and Z″″ are all carbon, and R′ and R″ represent —H, —OH, C₁-C₄ alkoxy or C₁-C₄ acyloxy or R′ and R″ taken together form an oxo, methano, or hydroxyimino group;

R₆, R₁₀, R₁₁, R₁₂ and R₁₃ each independently represent hydrogen, a lower alkyl having 1-4 carbons, halogen, nitro, —OR₇, —SR₇, —NR₇R₈ or —(CF)ₙCF₃, and exist only if the Z, Z′, Z″, Z′″, or Z″″ from which R₆, R₁₀, R₁₁, R₁₂ or R₁₃ originates is C, or R₆, R₁₀, R₁₁, R₁₂ and R₁₃ each independently represent hydrogen or a lower alkyl having 1-4 carbons if the Z, Z′, Z″, Z′″, or Z″″ from which R₆, R₁₀, R₁₁, R₁₂ or R₁₃ originates is N, and where one of R₆, R₁₀, R₁₁, R₁₂ or R₁₃ is X;

R₇ represents hydrogen or a lower alkyl having 1-6 carbons;

R₈ represents hydrogen or a lower alkyl having 1-6 carbons;

R₉ represents a lower alkyl having 1-4 carbons, phenyl, aromatic alkyl, or q-hydroxyphenyl, q-bromophenyl, q-chlorophenyl, q-fluorophenyl, or q-iodophenyl, where q=2-4;

R₁₄ represents hydrogen, a lower alkyl having 1-4 carbons, oxo, hydroxy, acyl having 1-4 carbons, halogen, thiol, or thioketone;

R₁₇ is hydrogen, lower alkyl having 1-8 carbons, alkenyl optionally substituted with halogen, acyl, —OR₇ or —SR₇, —R₉, alkyl carboxylic acid optionally substituted with halogen, acyl, —OR₇ or —SR₇, alkenyl carboxylic acid optionally substituted with halogen, acyl, —OR₇ or —SR₇, alkyl amine optionally substituted with halogen, acyl, —OR₇ or —SR₇, or alkenyl amine optionally substituted with halogen, acryl, —OR₇ or —SR₇;

R₁₈ represents hydrogen, a lower alkyl having 1-4 carbons, halogen, nitro, —OR₇, —SR₇, —NR₇R₈, or —CF)ₙCF₃;

X is —COOH, tetrazole, —PO₃H, —SO₃H, —CHO, —CH₂OH, —CONH₂, —COSH, —COOR₉, —COSR₉, —CONHR₉, or —COOW where W is a pharmaceutically acceptable salt, and wherein X can originate from any C or N on the ring;

Z, Z′, Z″, Z′″ and Z″″ each independently is C, S, O, N, or a pharmaceutically acceptable salt, provided that one or more of Z, Z′, Z″, Z′″ and Z″″ are not O or S if Z, Z′, Z″, Z′″ or Z″″ is attached by a double bond to one of Z, Z′, Z″, Z′″ or Z″″ or if one or more of Z, Z′, Z″, Z′″ or Z″″ is attached to one of Z, Z′, Z″, Z′″ or Z″″ that is O or S, and provided that one or more of Z, Z′, Z″, Z′″ and Z″″ are not N if one of Z, Z′, Z″, Z′″ and Z″″ is attached by a single bond to one of Z, Z′, Z″, Z′″ and Z″″ that is N;

n is 0 to 3; and

the dashed lines are optional double bonds.

The invention also includes the use of the compounds disclosed (e.g., RXR agonists) herein for the manufacture of a medicament for treating cachexia associated with one or more of the diseases, disorders or conditions named above.

The invention further includes pharmaceutical compositions for treating cachexia comprising a compound (e.g., an RXR agonist) disclosed herein.

### BRIEF DESCRIPTION OF THE DRAWINGS

FIG. 1 is a graph showing the actual body weight (in grams) of nude mice bearing H292 xenografts versus days post tumor transplant, with and without treatment by an RXR agonist compound in accordance with the invention.

FIG. 2 is a graph showing the percentage of survival of nude mice bearing H292 xenografts versus days post tumor transplant, with and without treatment by an RXR agonist compound in accordance with the invention.

FIG. 3 is a graph showing the actual body weight of severe combined immunodeficiency (SCID) mice bearing metastatic H446 tumors versus days post transplant, with and without treatment by an RXR agonist compound in accordance with the invention.

FIG. 4 is a graph showing the weight of the right gastrocnemius muscle of mice bearing H292 tumor xenograft 62 days after transplantation, with and without treatment by an RXR agonist compound in accordance with the invention.

FIG. 5 is a graph showing the average food intake of nude mice with and without H292 xenografts, and with and without treatment by an RXR agonist compound (Compound 1) in accordance with the invention.

FIG. 6 is a graph showing the actual body weight (in grams) of nude mice bearing H292 xenografts versus days post tumor transplant, with and without treatment by a RXR agonist compound (Compound 2) in accordance with the invention.

FIG. 7 is a graph showing the average food intake of nude mice bearing H292 xenografts with and without treatment by an RXR agonist compound (Compound 2) in accordance with the invention.

### DETAILED DESCRIPTION OF THE INVENTION

### Cachexia

Cachexia, which literally means ‘bad condition’, refers to involuntary weight loss, anorexia (loss of appetite), loss of protein and fat mass, gain in the proportion of body-water, and a variety of metabolic changes, which are associated with a primary disease, condition or disorder. The metabolic changes that can occur with cachexia include, for example, an elevation of resting energy expenditures (REEs) (Ann. Surg., 197: 152 (1983)), glucose intolerance and insulin resistance (Cancer Res., 44: 1718 (1984)), an increase in fat oxidation rates (Metabolism, 35: 304 (1986)) and whole body protein turnover (Cancer Res., 82: 42 (1998)). The pattern of weight loss in cachexia is different from normal starvation. For example, the normal adaptive response to nutrient deprivation is to draw on energy-dense lipid while sparing protein, resulting in loss of fat and relative preservation of lean body mass. In contrast, cachectic patients experience severe and incapacitating muscle wasting with relative sparing of adipose tissue.

Disease, conditions or disorders that are typically associated with cachexia include, but are not limited to, cancer, AIDS, liver cirrhosis, diabetes mellitus, chronic renal failure, chronic obstructive pulmonary disease, chronic cardiac failure, immune system diseases (e.g., rheumatoid arthritis and systemic lupus erythematosus), tuberculosis, cystic fibrosis, gastrointestinal disorders (e.g., irritable bowel syndrome and inflammatory bowel disease), Parkinson's disease, dementia, major depression, anorexia nervosa, an aged condition and sarcopenia. More typically, the disease, conditions or disorders that are associated with cachexia include, but are not limited to, cancer, AIDS, liver cirrhosis, chronic renal failure, chronic obstructive pulmonary disease, chronic cardiac failure, immune system diseases (e.g., rheumatoid arthritis and systemic lupus erythematosus), tuberculosis, cystic fibrosis, gastrointestinal disorders (e.g., irritable bowel syndrome and inflammatory bowel disease), Parkinson's disease, dementia, major depression, anorexia nervosa, an aged condition and sarcopenia. Cachexia is a strong independent risk factor for morbidity and mortality. For example, cancer cachexia occurs in about half of all cancer patients and is more common in patients with lung and upper gastronintestinal cancers (for a more detailed description see the publications: Nature Reviews Cancer, 2: 862 (2002); Proc. Natl. Acad. Sci. USA, 100: 5384 (2003); CA Cancer J. Clin., 52: 72 (2002)). Cancer patients with an involuntary 5% weight loss have a shorter median survival rate than patients with stable weight. Cancer patients with weight loss can respond poorly to chemotherapy and also can require increased chemotherapy treatments (Am. J. Med., 69: 491 (1980)). The fact that a large proportion of cancer patients have cachexia, coupled with the demonstrated relationship between cachexia and mortality, has provided impetus for the search into underlying mechanisms and therapies that might prevent or reverse cachexia and provide a model for identifying additional therapies.

Studies indicate that deregulation of neuroendocrine hormones, particularly catecholamines, glucagon, corticosterone, leptin and growth hormone are involved in the induction of cachexia (for reviews see Int. J. Cardiol., 85: 111 (2002); J. Nutrition, 129: 290S (1999)). More importantly, inappropriate production and release of cytokines such as TNF-α, interleukin-1, interleukin-6, interferon-γ, leukemia inhibitory factor, and ciliary neurotrophic factor, either alone or in combination, are able to cause the metabolic changes associated with cachexia and finally to induce wasting (for reviews see Drug Discov. Today, 8: 838 (2003); Int. J. Cardiol., 85: 73 (2002)). Recent studies indicate that the ubiquitin-proteasome proteolytic pathway plays a role in wasting of skeletal muscle and the intracellular events and transcription factors are also involved (Nature-Review-Cancer, 2:862-871 (2002)).

A variety of strategies have been tried to achieve these aims, which include (1) use of nutritional supplementation with improved diet, (2) administration of agents that can reduce energy expenditures, e.g., β-adrenergic blockers and nonsteroidal anti-inflammatory drugs such as COX inhibitors, (3) appetite stimulants, e.g., progesterone and cannabinoids, (4) anabolic stimulants, e.g., testosterone and IGF-1, (5) anticytokines, e.g., β-2 agonist such as clenbuterol and analogues, omega-3 fatty acids, melatonin, and thalidomide, and (5) miscellaneous agents, e.g., Ghrelin, anadamide, ponalrestat, ATP, cyclic plasma perfusion, IL-1 receptor agonist A, IL-15 and decoy nuclear factor κB (Current Oncology Reports, 4:264-274 (2002)). There are currently four approved drug products for the treatment of wasting and some of them are used for AIDS-related cachexia: Oxandrolone, Dronabinol, Megestrol acetate and growth hormone. (for a review, see J. Nutrition, 129: 303S (1999)).

Oxandrolone is an anabolic steroid being a synthetic derivative of testosterone. The indications for Oxandrolone include use as an adjunctive therapy to promote weight gain following weight loss after extensive surgery, chronic infections, or severe trauma; for patients with unexplained weight loss; and to offset protein catabolism associated with prolonged corticosteroid use. Dronabinol is an orally active cannabinoid first approved for the treatment of nausea and vomiting and were extended in 1992 to the treatment of anorexia associated with AIDS. The third drug approved for a wasting related indication was megestrol acetate, a synthetic progesterone derivative. It is approved for the treatment of anorexia, cachexia or weight loss in patients with AIDS and hormone-sensitive malignancies. Growth hormone has been approved for the treatment of AIDS wasting and cachexia. This drug received accelerated approval for wasting based on a positive change in lean body mass.

Despite of the numerous efforts in developing treatments for cachexia, few efficacious therapeutic solutions are known. In randomized clinical trials, dietary counseling and use of nutritional supplements have failed to ameliorate the symptoms of cachexia in chronically ill, nonmalignant patients (for reviews, see Am. J. Clin. Nutr., 74: 6 (2001); J. Nutrition, 129: S290 (1999)). Furthermore, artificial and aggressive feeding does not appear to have an impact on the overall survival of advanced cancer patients (J. Clin. Oncol., 2: 534 (1984)) and the global quality of life remains unaffected. Drugs that enhance appetite and anabolic therapies, despite the demonstrated efficacy in randomized clinical trials, do not have a major long-term impact on the vast majority of patients. For example, Dronabinol treatment was associated with improved appetite but had no effect on mood and body weight improvement (J. Clin. Oncol., 20: 567 (2002)). On the other hand, Oxandrolone treatment resulted in a moderate increase of body weight that might have represented primary edema (Proc. Am. Soc. Clin. Oncol., 21: 363a (2002)). Megestrol acetate treatment resulted in body weight gain of at least five pounds in AIDS as well as cancer patients (AIDS Res. Hum. Retrov., 13: 305 (1997); J. Clin. Oncol., 11: 762 (1993); Annals Oncol., 12: 289 (2001)). However, the primary body component that increased was fat, but not lean body mass.

Therefore, taken together, it is difficult to determine the actual clinical relevance, e.g., impact on morbidity, mortality, or quality of life, of the pharmacological therapies in cachectic patients. As such, there is a need for improved methods for the treatment of cachexia. In a preferred embodiment of the invention, the cachexia being treated is associated with one or more diseases, conditions and disorders selected from the group consisting of cancer, AIDS, liver cirrhosis, diabetes mellitus, chronic renal failure, chronic obstructive pulmonary disease, chronic cardiac failure, immune system diseases, tuberculosis, cystic fibrosis, gastrointestinal disorders, an aged condition and sarcopenia. In one particularly preferred embodiment, the cachexia is associated with one or more of AIDS, liver cirrhosis, diabetes mellitus, chronic renal failure, chronic obstructive pulmonary disease, chronic cardiac failure, immune system diseases, tuberculosis, cystic fibrosis, gastrointestinal disorders, an aged condition and sarcopenia. In another particularly preferred embodiment, the cachexia is associated with one or more of cancer, AIDS, liver cirrhosis, chronic renal failure, chronic obstructive pulmonary disease, chronic cardiac failure, immune system diseases, tuberculosis, cystic fibrosis, gastrointestinal disorders, an aged condition and sarcopenia. In yet another preferred embodiment, the cachexia is associated with one or more of AIDS, liver cirrhosis, chronic renal failure, chronic obstructive pulmonary disease, chronic cardiac failure, immune system diseases, tuberculosis, cystic fibrosis, gastrointestinal disorders, an aged condition and sarcopenia. In a specific embodiment, the cachexia is associated with cancer. In another specific embodiment, the cachexia is associated with AIDS.

### Cancer

As used herein, cancer refers to tumors, neoplasms, carcinomas, sarcomas, leukemias, lymphomas and the like. For example, cancers include, but are not limited to, leukemias and lymphomas such as cutaneous T-cell lymphoma (CTCL), non-cutaneous peripheral T-cell lymphoma, lymphomas associated with human T-cell lymphotropic virus (HTLV), for example, adult T-cell leukemia/lymphoma (ATLL), acute lymphocytic leukemia, acute nonlymphocytic leukemias, chronic lymphocytic leukemia, chronic myelogenous leukemia, Hodgkin's Disease, non-Hodgkin's lymphomas, and multiple myeloma, childhood solid tumors such as brain tumors, neuroblastoma, retinoblastoma, Wilms' Tumor, bone tumors, and soft-tissue sarcomas, common solid tumors of adults such as head and neck cancers (e.g., oral, laryngeal and esophageal), genitourinary cancers (e.g., prostate, bladder, renal, uterine, ovarian, testicular, rectal and colon), lung cancer, breast cancer, pancreatic cancer, melanoma and other skin cancers, stomach cancer, brain tumors, liver cancer, biliary cancer, gastrointestinal cancers (e.g., small intestinal, gastric) and thyroid cancer.

### Retinoid X Receptor (RXR) Agonists

There are two main types of retinoid receptors that have been identified in mammals (and other organisms). The two main types or families of receptors are respectively designated the Retinoid Acid Receptors (RARs) and Retinoid X Receptors (RXRs).

The Retinoid X Receptor (RXR) is a member of the nuclear hormone receptor family of proteins. RXR contains two signature domains of nuclear receptor family proteins, the DNA-binding domain and ligand binding domain (LBD). RXR is a ligand-dependent transcription factor. The endogenous ligand for RXR is 9-cis retinoic acid. RXR plays an important role in many fundamental biological processes such as reproduction, cellular differentiation, bone development, hematopoiesis and pattern formation during embryogenesis (Mangelsdorf, D. J. et al., Cell, 83: 841-850 (1995)). RXR is also implicated in some pathological conditions as neoplastic formation and it is a potential target for cancer therapy (Nagy, L., et al., Cell Death and Diff., 5: 11-19 (1998)).

The mammalian RXR includes at least three distinct genes, RXR_{α}, RXR_{β} and RXR_{γ} (RXR alpha, beta and gamma) which give rise to a large number of protein products through differential promoter usage and alternative splicing. Compounds useful in treating cachexia can be agonists for the RXR_{α}, RXR_{β} or RXR_{γ} receptor. Besides acting as a homodimer, RXR plays a central role in regulating the activity of other nuclear hormone receptors by acting as a partner for heterodimers. RXR forms a functional heterodimer with retinoic acid receptor (RAR), thyroid hormone receptor, vitamin D receptor, NGFI-B and many other nuclear receptors. The different binding partners of the RXR render a different DNA-binding specificity of the heterodimer.

As used herein, RXR refers to naturally occurring RXRs (e.g., mammalian RXRs (e.g., human (Homo sapien) RXRs, murine (e.g., rat, mouse) RXRs) and to proteins having an amino acid sequence which is the same as that of a corresponding naturally occurring RXR (e.g., recombinant proteins). The term includes naturally occurring variants, such as polymorphic or allelic variants and splice variants.

As used herein, the term an RXR agonist refers to a substance (e.g., a molecule, a compound) which promotes (induces or enhances) at least one function characteristic of an RXR. In one embodiment, the RXR agonist binds the RXR. In certain embodiments, the agonist is a partial agonist. Partial agonist, as used herein, refers to an agonist which no matter how high of a concentration is used, is unable to produce maximal activation of the RXR. Some RXR agonists may have mixed agonist-antagonist activity.

An RXR agonist can be identified and activity assessed by any suitable method. For example a chimeric receptor transactivation assay that tests for agonist-like activity in the RAR_{α}, RAR_{β}, RAR_{γ}, RXR_{α} receptor subtypes, and that is based on work published by Feigner P. L. and Holm M. Focus, 112, (1989), is described in detail in U.S. Pat. No. 5,455,265, which is hereby incorporated by reference. In addition, a holoreceptor transactivation assay and a ligand binding assay that measure the antagonist/agonist like activity of the compounds of the invention, or their ability to bind to the several retinoid receptor subtypes, respectively, are described in WO 93/11755 (particularly on pages 30-33 and 37-41) published on Jun. 24, 1993, the content of which is also incorporated herein by reference. A detailed experimental procedure for holoreceptor transactivations has been described by Heyman et al., Cell 68: 397-406, (1992); Allegretto et al., J. Biol. Chem, 268: 26625-26633, and Mangelsdorf et al., The Retinoids: Biology, Chemistry and Medicine, pp 319-349, Raven Press Ltd., New York, which are incorporated herein by reference. The results obtained in this assay and the chimeric receptor transactivation assay, are expressed as EC₅₀ values. Still another transactivation assay, the “PGR assay” is described in Klein et al., J. Biol. Chem. 271: 22692-22696 (1996), which is incorporated herein by reference.

In a particular embodiment, the RXR agonists are described, for example, in U.S. Pat. Nos. 6,403,638; 6,388,105; 6,313,163; 6,147,224; 6,114,533; 6,048,873; 6,048,873; 6,034,242; 5,917,082; 5,817,836; 5,780,647; 5,675,033; 5,663,367; 6,320,074; 6,162,815; 5,977,125; 5,801,253; 6,326,397 and 6,043,279 the entire contents of which are expressly incorporated herein by reference. RXR agonist compounds that can be administered in accordance with the present invention are also described, for example, in the following PCT Published Patent Applications: WO 97/12853; WO 01/19770; WO 00/53562; WO 01/70668 and WO/02/071827, the entire contents of which are expressly incorporated herein by reference.

Preferably, RXR agonists having the structures described in U.S. Pat. Nos. 5,675,033, 5,917,082 and 6,320,074 are used in the pharmaceutical compositions and methods of the present invention. Even more preferably, RXR agonist compounds of U.S. Pat. Nos. 5,675,033 and 5,917,082 are used.

Examples of RXR agonist compounds disclosed in U.S. Pat. Nos. 5,675,033 and 5,917,082 are represented by Structural Formula (I):

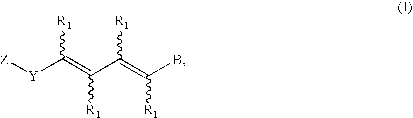

where:

Z is represented by Structural Formula (II) or Structural Formula (III)

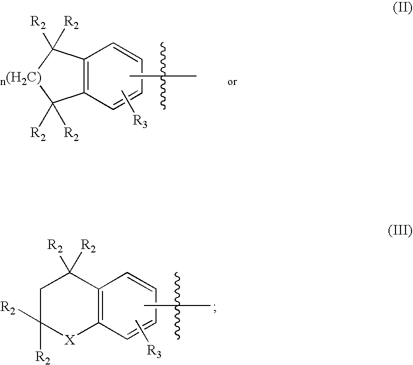

Y is cycloalkyl of 3 to 8 carbons or cycloalkenyl of 5 to 8 carbons optionally substituted with one or two R₄ groups, or Y is selected from phenyl, pyridyl, thienyl, furyl, pyrrolyl, pyridazinyl, pyrimidinyl, pyrazinyl, thiazolyl, oxazolyl, and imidazolyl, said groups being optionally substituted with one or two R₄ groups, and wherein Y is substituted by the Z and —CR₁═CR₁—CR₁═CR₁— groups on adjacent carbons; preferably, Y is cyclopropyl, phenyl, pyridyl, thienyl or furyl; more preferably, Y is cyclopropyl or phenyl; and even more preferably, Y is a cyclopropyl substituted with a methyl group at the carbon atom nearest to Z, thereby forming a quaternary carbon;

X is S, O, or NR₅;

n is 1 or 2;

R₁ and R₂ independently are H, lower alkyl or fluoroalkyl; preferably, R₁ is H or methyl;

R₃ is hydrogen, lower alkyl, alkylamino, dialkylamino, cyano, Cl or Br;

R₄ is lower alkyl, fluoroalkyl or halogen;

R₅ is H or lower alkyl;

B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl; preferably, B is —COOH or a pharmaceutically acceptable salt thereof, —COOR₈ or —CONR₉R₁₀;

R₇ is an alkyl, cycloalkyl or alkenyl group containing 1 to 5 carbons;

R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl;

R₉ and R₁₀ independently are hydrogen, an alkyl group of 1 to 10 carbons, or a cycloalkyl group of 5 to 10 carbons, or phenyl or lower alkylphenyl;

R₁₁ is lower alkyl, phenyl or lower alkylphenyl;

R₁₂ is lower alkyl; and

R₁₃ is divalent alkyl radical of 2 to 5 carbons.

In one preferred embodiment, Z is represented by Structural Formula (II) and n is 2. In another preferred embodiment, Z is represented by Structural Formula (III) and X is S or O.

In a particular embodiment, Z is represented by Structural Formula (II) or (III); Y is selected from pyridyl, pyrrolyl, pyridazinyl, pyrimidinyl, pyrazinyl, thiazolyl, oxazolyl, and imidazolyl, said groups being optionally substituted with one or two R₄ groups, and wherein Y is substituted by the Z and —CR₁═CR₁—CR₁═CR₁— groups on adjacent carbons; X is NR₅; n is 1 or 2; R₁ and R₂ independently are —H, lower alkyl or fluoroalkyl; R₃ is hydrogen, lower alkyl, alkylamino, dialkylamino, cyano, —Cl or —Br; R₄ is lower alkyl, fluoroalkyl or halogen; R₅ is —H or lower alkyl; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl; R₇ is an alkyl, cycloalkyl or alkenyl group containing 1 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is a divalent alkyl radical of 2 to 5 carbons.

In another particular embodiment, Z is represented by Structural Formula (III); Y is thienyl or furyl, said thienyl or furyl groups being optionally substituted with one or two R₄ groups, and wherein Y is substituted by the Z and —CR₁═CR₁—CR₁═CR₁— groups on adjacent carbons; X is NR₅; n is 1 or 2; R₁ and R₂ independently are —H, lower alkyl or fluoroalkyl; R₃ is hydrogen, lower alkyl, alkylamino, dialkylamino, cyano, —Cl or —Br; R₄ is lower alkyl, fluoroalkyl or halogen; R₅ is H or lower alkyl; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl; R₇ is an alkyl, cycloalkyl or alkenyl group containing 1 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is a divalent alkyl radical of 2 to 5 carbons.

In yet another particular embodiment, Z is represented by Strucutural Formula (III); Y is cycloalkyl of 3 to 8 carbons or cycloalkenyl of 5 to 8 carbons optionally substituted with one or two R₄ groups, or Y is selected from phenyl, pyridyl, thienyl, furyl, pyrrolyl, pyridazinyl, pyrimidinyl, pyrazinyl, thiazolyl, oxazolyl, and imidazolyl, said groups being optionally substituted with one or two R₄ groups, and wherein Y is substituted by the Z and —CR₁═CR₁—CR₁═CR₁— groups on adjacent carbons; X is S or O; n is 1 or 2; R₁ and R₂ independently are H, lower alkyl or fluoroalkyl; R₃ is hydrogen, lower alkyl, alkylamino, dialkylamino, cyano, Cl or Br; R₄ is lower alkyl, fluoroalkyl or halogen; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl; R₇ is an alkyl, cycloalkyl or alkenyl group containing 1 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is divalent alkyl radical of 2 to 5 carbons.

In a further particular embodiment of compounds represented by Structural Formula (I), Z is represented by Structural Formula (II); Y is selected from thienyl or furyl, said groups being optionally substituted with one or two R₄ groups, and wherein Y is substituted by the Z and —CR₁═CR₁—CR₁═CR₁— groups on adjacent carbons; n is 1 or 2; R₁ and R₂ independently are H, lower alkyl or fluoroalkyl; R₃ is hydrogen, lower alkyl, alkylamino, dialkylamino, cyano, Cl or Br; R₄ is lower alkyl, fluoroalkyl or halogen; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl; R₇ is an alkyl, cycloalkyl or alkenyl group containing 1 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is divalent alkyl radical of 2 to 5 carbons.

Another group of compounds represented by Structural Formula (I) include those where Z is represented by Structural Formula (III); Y is cycloalkyl of 3 to 8 carbons or cycloalkenyl of 5 to 8 carbons optionally substituted with one or two R₄ groups, or Y is phenyl, said groups being optionally substituted with one or two R₄ groups, and wherein Y is substituted by the Z and —CR₁═CR₁—CR₁═CR₁— groups on adjacent carbons; X is NR₅; R₁ and R₂ independently are —H, lower alkyl or fluoroalkyl; R₃ is hydrogen, lower alkyl, alkylamino, dialkylamino, cyano, —Cl or —Br; R₄ is lower alkyl, fluoroalkyl or halogen; R₅ is —H or lower alkyl; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl; R₇ is an alkyl, cycloalkyl or alkenyl group containing 1 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is divalent alkyl radical of 2 to 5 carbons.

Yet another group of compounds represented by Structural Formula (I) include those where Z is represented by Structural Formula (III); Y is cyclopropyl, said Y group being optionally substituted with one or two R₄ groups, and wherein Y is substituted by the Z and —CR₁═CR₁—CR₁═CR₁— groups on adjacent carbons; X is NR₅; R₁ and R₂ independently are H, lower alkyl or fluoroalkyl; R₃ is hydrogen, lower alkyl, alkylamino, dialkylamino, cyano, —Cl or —Br; R₄ is lower alkyl, fluoroalkyl or halogen; R₅ is —H or lower alkyl; B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri(lower alkyl)silyl; R₇ is an alkyl of 1 to 5 carbons, cycloalkyl of 3 to 5 carbons or alkenyl group containing 2 to 5 carbons; R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; R₉ and R₁₀ independently are hydrogen, an alkyl group of 1 to 10 carbons, or a cycloalkyl group of 5 to 10 carbons, or phenyl or lower alkylphenyl; R₁₁ is lower alkyl, phenyl or lower alkylphenyl; R₁₂ is lower alkyl; and R₁₃ is divalent alkyl radical of 2 to 5 carbons.

Still more preferably, compounds of the general structure shown by Structural Formula (IV) are used:

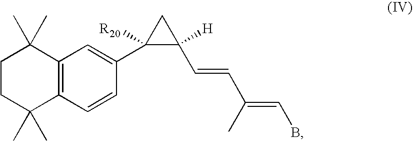

where R₂₀ is alkyl of 1 to 6 carbons, and B is —COOH, or —COOR₂₁ where R₂₁ is alkyl of 1 to 6 carbons, or a pharmaceutically acceptable salt of said compound.

Compounds 1, 2 and 3, the chemical formulas of which are shown below, are specific examples of RXR agonists that can be used, either as a free acid or as a pharmaceutically acceptable salt, in accordance with the present invention to treat mammals, including human beings, to prevent, inhibit or reduce (partially or completely) cachexia. Among all RXR agonists, Compounds 1 and 2 are presently the most preferred to be used in the present invention. Compounds 1 and 2 are within the scope of Structural Formula (IV).

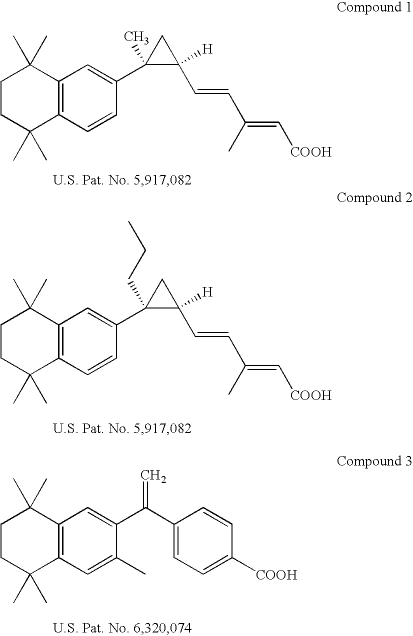

Compounds 1 and 2 can be obtained in accordance with the synthetic procedures described in U.S. Pat. No. 5,917,082. Compound 3 can be obtained in accordance with the synthetic procedure described in U.S. Pat. No. 6,320,714. The entire contents of both of these patents are expressly incorporated herein by reference.

Further preferred compounds disclosed by U.S. Pat. No. 5,917,082 are represented by Structural Formula (V):

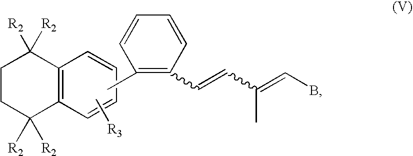

where:

R₂ is hydrogen or lower alkyl;

R₃ is hydrogen or lower alkyl;

B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri-lower alkylsilyl;

R₇ is an alkyl, cycloalkyl or alkenyl group containing 1 to 5 carbons;

R₈ is an alkyl group of 1 to 10 carbons, a cycloalkyl group of 5 to 10 carbons or trimethylsilylalkyl where the alkyl group has 1 to 10 carbons, or R₈ is phenyl or lower alkylphenyl;

R₉ and R₁₀ independently are hydrogen, an alkyl group of 1 to 10 carbons, or a cycloalkyl group of 5 to 10 carbons, or phenyl or lower alkylphenyl;

R₁₁ is lower alkyl, phenyl or lower alkylphenyl;

R₁₂ is lower alkyl; and

R₁₃ is divalent alkyl radical of 2 to 5 carbons.

Other preferred compounds encompassed by U.S. Pat. No. 5,917,082 are represented by Structural Formula (VI):

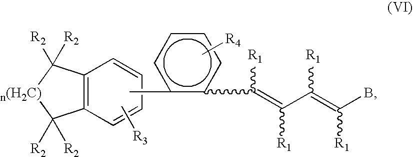

where:

n is 1 or 2;

R₁ and R₂ independently are H, lower alkyl or fluoroalkyl;

R₃ is hydrogen, lower alkyl, —Cl or —Br;

R₄ is H, lower alkyl, fluoroalkyl or halogen;

B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri-lower alkylsilyl;

R₇ is an alkyl, cycloalkyl or alkenyl group containing 1 to 5 carbons;

R₈ is an alkyl group of 1 to 10 carbons, a cycloalkyl group of 5 to 10 carbons or trimethylsilylalkyl where the alkyl group has 1 to 10 carbons, or R₈ is phenyl or lower alkylphenyl;

R₉ and R₁₀ independently are hydrogen, an alkyl group of 1 to 10 carbons, or a cycloalkyl group of 5 to 10 carbons, or phenyl or lower alkylphenyl;

R₁₁ is lower alkyl, phenyl or lower alkylphenyl;

R₁₂ is lower alkyl; and

R₁₃ is divalent alkyl radical of 2 to 5 carbons.

Another group of preferred compounds disclosed by U.S. Pat. No. 5,917,082 is represented by Structural Formula (VII):

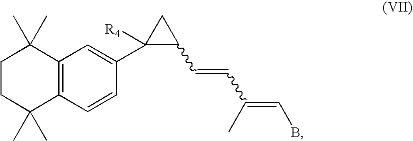

where:

R₄ is lower alkyl of 1 to 6 carbons;

B is —COOH or —COOR₈; and

R₈ is lower alkyl of 1 to 6 carbons; and the configuration about the cyclopropane ring is cis, and the configuration about the double bonds in the pentadienoic acid or ester chain attached to the cyclopropane ring is trans in each of said double bonds, and pharmaceutically acceptable salts thereof.

Yet another group of preferred compounds disclosed by U.S. Pat. No. 5,917,082 is represented by Structural Formula (VIII):

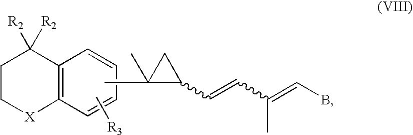

wherein:

X is S or O; alternatively, X is NR₅;

R₂ is hydrogen or lower alkyl;

R₃ is hydrogen or lower alkyl;

R₅ is hydrogen or lower alkyl;

B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri-lower alkylsilyl;

R₇ is an alkyl, cycloalkyl or alkenyl group containing 1 to 5 carbons, such as an alkyl of 1 to 5 carbons, a cycloalkyl of 3 to 5 carbons or an alkenyl group containing 2 to 5 carbons;

R₈ is an alkyl group of 1 to 10 carbons, a cycloalkyl group of 5 to 10 carbons or trimethylsilylalkyl where the alkyl group has 1 to 10 carbons, or R₈ is phenyl or lower alkylphenyl;

R₉ and R₁₀ independently are hydrogen, an alkyl group of 1 to 10 carbons, or a cycloalkyl group of 5 to 10 carbons, or phenyl or lower alkylphenyl;

R₁₁ is lower alkyl, phenyl or lower alkylphenyl;

R₁₂ is lower alkyl; and

R₁₃ is divalent alkyl radical of 2 to 5 carbons.

Particularly preferred compounds encompassed by Structural Formula (I) are represented by Structural Formulas (IX), (X) and (XI):

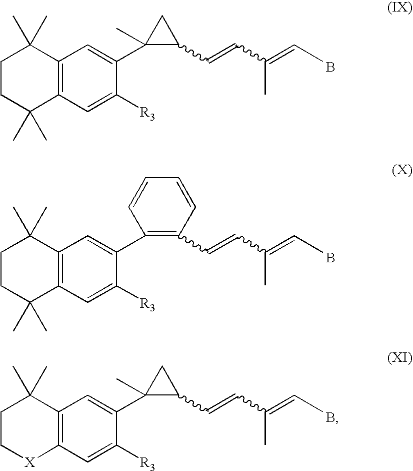

where:

B is —COOH or —COOR₈;

R₃ is hydrogen, lower alkyl, —Cl or —Br;

R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl; and

X is S or O.

When the compound is represented by Structural Formula (IX), R₃ is preferably H or methyl and B is preferably —COOH or —COOCH₂CH₃. Particularly preferred compounds are represented by Structural Formula (IX), wherein R₃ is —H, B is —COOH or —COOR, and R is lower alkyl of 1 to 6 carbons, and pharmaceutically acceptable salts thereof.

When the compound is represented by Structural Formula (X), it is preferred that R₃ is —H and B is —COOH or —COOCH₂CH₃.

When the compound is represented by Structural Formula (XI), it is preferred that R₃ is —H, B is —COOH or —COOCH₂CH₃ and X is O or S.

Additional compounds useful for treating cachexia, without limitation to the disease, disorder or condition with the cachexia is associated, are shown below.

One group of compounds useful in treating cachexia is represented by Structural Formulas (XIII), (XIV) or (XV):

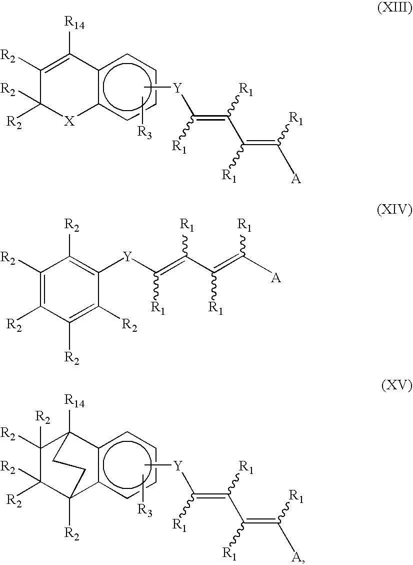

where:

X is O, S, or (CR₁R₁)ₙ;

n is 0, 1 or 2;

Y is a bivalent radical having Structural Formula (XVI) or Structural Formula (XVII) where p is an integer from 1 to 4:

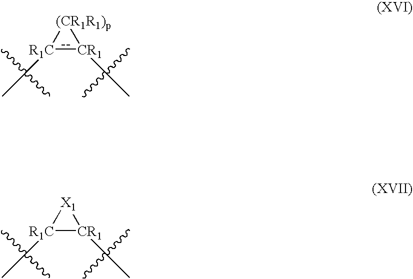

or Y is a bivalent aryl or 5 or 6 membered heteroaryl radical having 1 to 3 heteroatoms selected from N, S and O, said aryl or heteroaryl groups being unsubstituted, or substituted with 1 to 3 C₁₋₆ alkyl or with 1 to 3 C₁₋₆ fluoroalkyl groups;

X is O, S or NH;

R₁ is independently —H, lower alkyl of 1 to 6 carbons, or lower fluoroalkyl of 1 to 6 carbons;

R₂ is independently —H, lower alkyl of 1 to 6 carbons, —OR₁, 1-adamantyl, or lower fluoroalkyl of 1 to 6 carbons, or the two R₂ groups jointly represent an oxo group;

R₃ is hydrogen, lower alkyl of 1 to 6 carbons, —OR₁, fluoro substituted lower alkyl of 1 to 6 carbons or halogen, —NO₂, —NH₂, —NHCO(C₁-C₆)alkyl, or —NHCO(C₁-C₆)alkenyl;

A is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CH(OR₁₃O), —COR₇, —CR₇(OR₁₂)₂, —CR₇(OR₁₃O), or —Si(C₁₋₆ alkyl)₃;

R₇ is an alkyl, cycloalkyl or alkenyl group containing 1 to 5 carbons;

R₈ is an alkyl group of 1 to 10 carbons or (trimethylsilyl)alkyl where the alkyl group has 1 to 10 carbons, or a cycloalkyl group of 5 to 10 carbons, or R₈ is phenyl or lower alkylphenyl;

R₉ and R₁₀ independently are hydrogen, an alkyl group of 1 to 10 carbons, or a cycloalkyl group of 5-10 carbons, or phenyl, hydroxyphenyl or lower alkylphenyl;

R₁₁ is lower alkyl, phenyl or lower alkylphenyl;

R₁₂ is lower alkyl;

R₁₃ is divalent alkyl radical of 2-5 carbons; and

R₁₄ is alkyl of 1 to 10 carbons, fluoro-substituted alkyl of 1 to 10 carbons, alkenyl of 2 to 10 carbons and having 1 to 3 double bonds, alkynyl having 2 to 10 carbons and 1 to 3 triple bonds, carbocyclic aryl selected from the group consisting of phenyl, C₁-C₁₀-alkylphenyl, naphthyl, C₁-C₁₀-alkylnaphthyl, phenyl-C₁-C₁₀ alkyl, naphthyl-C₁-C₁₀ alkyl, C₁-C₁₀-alkenylphenyl having 1 to 3 double bonds, C₁-C₁₀-alkynylphenyl having 1 to 3 triple bonds, phenyl-C₁-C₁₀ alkenyl having 1 to 3 double bonds, phenyl-C₁-C₁₀ alkynyl having 1 to 3 triple bonds, hydroxy alkyl of 1 to 10 carbons, hydroxyalkenyl having 2 to 10 carbons and 1 to 3 double bonds, hydroxyalkynyl having 2 to 10 carbons and 1 to 3 triple bonds, acyloxyalkyl of 1 to 10 carbons, acyloxyalkenyl having 2 to 10 carbons and 1 to 3 double bonds, or acyloxyalkynyl of 2 to 10 carbons and 1 to 3 triple bonds where the acyl group is represented by COR₈, or R₁₄ is a 5 or 6 membered heteroaryl group having 1 to 3 heteroatoms, said heteroatoms being selected from a group consisting of O, S, and N, said heteroaryl group being unsubstituted or substituted with a C₁ to C₁₀ alkyl group, with a C₁ to C₁₀ fluoroalkyl group, or with halogen, and the dashed line in Structural Formula (XVI) represents a bond or absence of a bond.

Another group of compounds suitable for treating cachexia is represented by Structural Formula (XVIII):

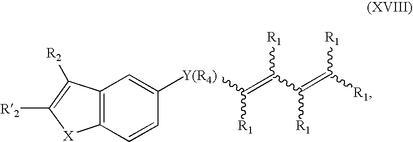

wherein:

X is O, NR′ or S;

R′ is alkyl of 1 to 6 carbons;

Y is a bivalent cyclopropyl radical optionally substituted with one or two R₄ groups, or Y is a bivalent aryl or 5 or 6 membered heteroaryl radical having 1 to 3 heteroatoms selected from N, S and O, said aryl or heteroaryl groups optionally substituted with 1 to 4 R₄ groups;

R₁ is independently —H, alkyl of 1 to 6 carbons, or fluoroalkyl of 1 to 6 carbons;

R₂ is alkyl of 1 to 8 carbons, or fluoroalkyl of 1 to 8 carbons;

R′₂ is alkyl of 1 to 8 carbons, or fluoroalkyl of 1 to 8 carbons;

R₃ is hydrogen, alkyl of 1 to 6 carbons, fluoro substituted alkyl of 1 to 6 carbons, halogen, alkoxy of 1 to 8 carbons, or alkylthio of 1 to 6 carbons, —NO₂, —NH₂, —NHCO(C₁-C₆)alkyl, —NHCO(C₁-C₆)alkenyl, —NR₁H or —N(R₁)₂, benzyloxy or C₁-C₆ alkyl-substituted benzyloxy;

R₄ is —H or alkyl of 1 to 6 carbons, or fluoro substituted alkyl of 1 to 6 carbons;

m is an integer having the values of 0 to 3, and

B is —COOH or a pharmaceutically acceptable salt thereof, —COOR₈, —COOCH₂COR₇, —CONR₉R₁₀, —CH₂OH, —CH₂OR₁₁, —CH₂OCOR₁₁, —CHO, —CH(OR₁₂)₂, —CH(OR₁₃O), —COR₇, —CR₇(OR₁₂)₂, —CR₇(OR₁₃O),

R₇ is an alkyl, cycloalkyl or alkenyl group containing 1 to 5 carbons;

R₈ is an alkyl group of 1 to 10 carbons or (trimethylsilyl)alkyl where the alkyl group has 1 to 10 carbons, or a group of 5 to 10 phenyl or lower alkylphenyl;

R₉ and R₁₀ independently are hydrogen, an alkyl group of 1 to 10 carbons, or a cycloalkyl group of 5-10 carbons, or phenyl, hydroxyphenyl or lower alkylphenyl;

R₁₁ is lower alkyl, phenyl or lower alkylphenyl;

R₁₂ is lower alkyl; and

R₁₃ is divalent alkyl radical of 2-5 carbons.

Yet another group of compounds useful for treating cachexia is represented by Structural Formula (XIX):

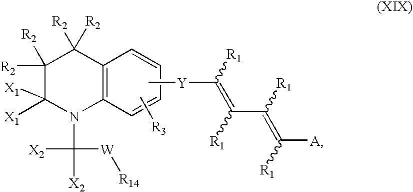

wherein:

Y is a bivalent radical having Formula (a) or Formula (b):

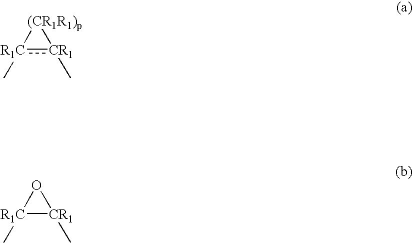

or Y is a bivalent aryl or 5 or 6 membered heteroaryl radical having 1 to 3 heteroatoms selected from N, S and O, said aryl or heteroaryl groups being unsubstituted, or substituted with 1 to 3 C₁₋₆ alkyl or with 1 to 3 C₁₋₆ fluoroalkyl groups;

p is an integer from 1 to 4;

the two X₁ groups jointly represent an oxo or thione function, or X₁ is independently selected from H or alkyl of 1 to 6 carbons;

the two X₂ groups jointly represent an oxo or a thione function, or X₂ is independently selected from H or alkyl of 1 to 6 carbons, with the proviso that one of the joint X₁ grouping or of the joint X₂ grouping represents an oxo or a thione function;

W is H, O, C(R₁)₂, phenyl, naphthyl, or 5 or 6 membered heteroaryl group having 1 to 3 heteroatoms, said heteroatoms being selected from a group consisting of O, S, and N, said phenyl, naphthyl or heteroaryl groups being unsubstituted or substituted with a C₁ to C₁₀ alkyl group, with a C₁ to C₁₀ fluoroalkyl group, or with halogen;

R₁ is independently —H, lower alkyl of 1 to 6 carbons, or lower fluoroalkyl of 1 to 6 carbons;

R₂ is independently —H, lower alkyl of 1 to 6 carbons, or lower fluoroalkyl of 1 to 6 carbons;

R₃ is hydrogen, lower alkyl of 1 to 6 carbons, —OR₁, fluoro substituted lower alkyl of 1 to 6 carbons or halogen, —NO₂, —NH₂, —NHCO(C₁-C₆ alkyl, or vNHCO(C₁-C₆)alkenyl;

A is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CH(OR₁₃O), —COR₇, —CR₇(OR₁₂)₂, —CR₇(OR₁₃O), or —Si(C₁₋₆ alkyl)₃;

R₇ is an alkyl, cycloalkyl or alkenyl group containing 1 to 5 carbons,

R₈ is an alkyl group of 1 to 10 carbons or (trimethylsilyl)alkyl where the alkyl group has 1 to 10 carbons, or a cycloalkyl group of 5 to 10 carbons, or R₈ is phenyl or lower alkylphenyl;

R₉ and R₁₀ independently are hydrogen, an alkyl group of 1 to 10 carbons, or a cycloalkyl group of 5-10 carbons, or phenyl, hydroxyphenyl or lower alkylphenyl;

R₁₁ is lower alkyl, phenyl or lower alkylphenyl;

R₁₂ is lower alkyl;

R₁₃ is divalent alkyl radical of 2-5 carbons;

R₁₄ is H, alkyl of 1 to 10 carbons, fluoro-substituted alkyl of 1 to 10 carbons, alkenyl of 2 to 10 carbons and having 1 to 3 double bonds, alkynyl having 2 to 10 carbons and 1 to 3 triple bonds, carbocyclic aryl selected from the group consisting of phenyl, C₁-C₁₀-alkylphenyl, naphthyl, C₁-C₁₀-alkylnaphthyl, phenyl-C₁-C₁₀ alkyl, naphthyl-C₁-C₁₀-alkyl, C₁-C₁₀-alkenylphenyl having 1 to 3 double bonds, C₁-C₁₀-alkynylphenyl having 1 to 3 triple bonds, phenyl-C₁-C₁₀ alkenyl having 1 to 3 double bonds, phenyl-C₁-C₁₀ alkynyl having 1 to 3 triple bonds, hydroxy alkyl of 1 to 10 carbons, hydroxyalkenyl having 2 to 10 carbons and 1 to 3 double bonds, hydroxyalkynyl having 2 to 10 carbons and 1 to 3 triple bonds, acyloxyalkyl of 1 to 10 carbons, acyloxyalkenyl having 2 to 10 carbons and 1 to 3 double bonds, or acyloxyalkynyl of 2 to 10 carbons and 1 to 3 triple bonds where the acyl group is represented by COR₈, or R₁₄ is a 5 or 6 membered heteroaryl group having 1 to 3 heteroatoms, said heteroatoms being selected from a group consisting of O, S, and N, said carbocyclic aryl and heteroaryl groups being unsubstituted or substituted with a C₁ to C₁₀ alkyl group, with a C₁ to C₁₀ fluoroalkyl group, or with halogen;

and the dashed line in Formula (a) represents a bond or absence of a bond, provided that when the dashed line represents a bond then there are no R₁ substituents on the carbons connected by said bond.

A further group of compounds suitable for treating cachexia is represented by Structural Formula (XX):

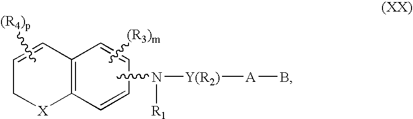

wherein:

X is O, S, or C(R)₂;

R is —H or alkyl of 1 to 6 carbons;

R₁ is —H, alkyl of 1 to 10 carbons, alkenyl of 2 to 6 carbons, phenyl-C₁-C₆ alkyl, or C₁-C₆-alkylphenyl;

R₂ is —H, alkyl of 1 to 6 carbons, —F, —Cl, —Br, —I, —CF₃, fluoro substituted alkyl of 1 to 6 carbons, alkoxy of 1 to 6 carbons, or alkylthio of 1 to 6 carbons;

R₃ is independently alkyl of 1 to 6 carbons, —F, —Cl, —Br, —I, —CF₃, fluoro substituted alkyl of 1 to 6 carbons, —OH, —SH, alkoxy of 1 to 6 carbons, fluoroalkoxy of 1 to 6 carbons, alkylthio of 1 to 6 carbons, benxyloxy, C₁-C₆ alkyl substituted benzyloxy, halogen substituted benzyloxy, phenyloxy, C₁-C₆ alkyl substituted phenyloxy, or halogen substituted phenyloxy;

R₄ is independently —H, alkyl of 1 to 6 carbons, or —F;

Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups; m is an integer having the values 0 to 3;

p is an integer having the values 0 to 4;

A is —(CH₂)_{q}— where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;

B is hydrogen, —COOH, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri-lower alkylsilyl;

R₇ is an alkyl, cycloalkyl or alkenyl group containing 1 to 5 carbons,

R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl;

R₉ and R₁₀ independently are hydrogen, an alkyl group of 1 to 10 carbons, or a cycloalkyl group of 5-10 carbons, or phenyl or lower alkylphenyl;

R₁₁ is lower alkyl, phenyl or lower alkylphenyl;

R₁₂ is lower alkyl; and

R₁₃ is divalent alkyl radical of 2-5 carbons, and pharmaceutically acceptable salts thereof.

Another group of compounds for treating cachexia is represented by Structural Formulas (XXI), (XXII), (XXIII), (XXIV), (XXV), (XXVI), (XXVII), (XXVIIa) or (XXVIIIb):

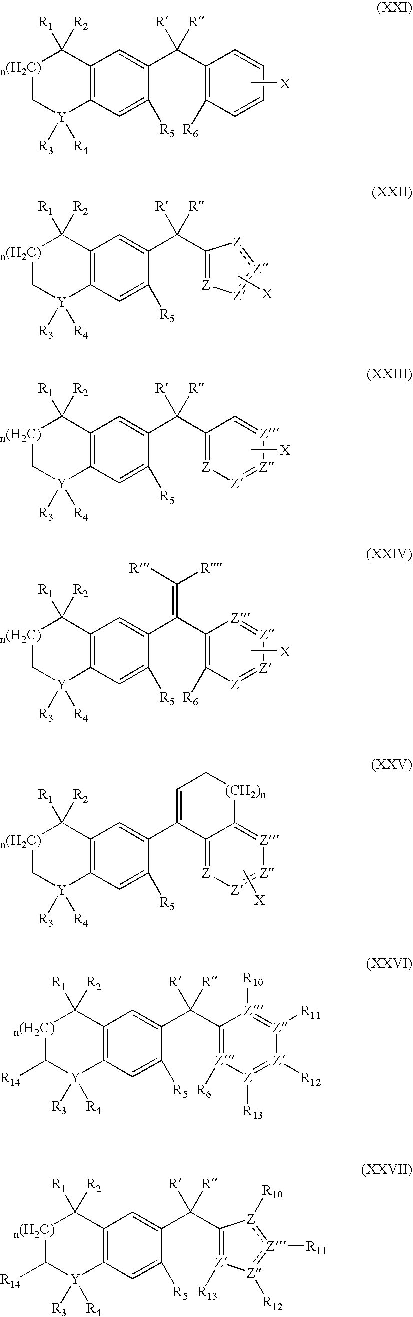

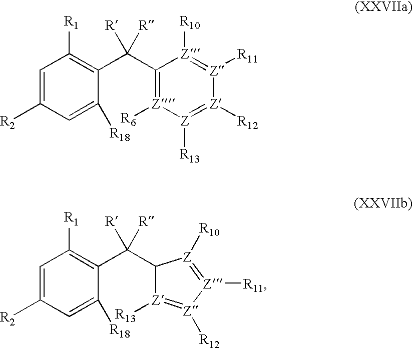

wherein:

R₁ and R₂ each independently is hydrogen or lower alkyl or acyl having 1-4 carbon atoms;

Y is C, O, S, N, CHOH, CO, SO, SO₂, or a pharmaceutically acceptable salt;

R₃ is hydrogen or lower alkyl having 1-4 carbon atoms where Y is C or N;

R₄ is hydrogen or lower alkyl having 1-4 carbon atoms when Y is C, R₄ does not exist if Y is N, or neither R₃ or R₄ exist if Y is S, O, CHOH, CO, SO, or SO₂;

R′ and R″ are hydrogen, lower alkyl or acyl having 1-4 carbon atoms, —OH, alkoxy having 1-4 carbon atoms, thiol or thioether, or amino, or R′ or R″ taken together form an oxo(keto), methano, thioketo, HO—N═, NC—N═, (R₇R₈)N—N═, R₁₇O—N═, R₁₇N═, epoxy, cyclopropyl, or cycloalkyl group and wherein the epoxy, cyclopropyl, and cycloalkyl groups are optionally substituted with lower alkyl having 1-4 carbons or halogen;

R′″ and R″″ are hydrogen, halogen, lower alkyl or acyl having 1-4 carbon atoms, alkylamino, or R′″ and R″″ taken together form a cycloalkyl group having 3-10 carbons, and wherein the cycloalkyl group can be substituted with lower alkyl having 1-4 carbons or halogen;

R₅ is hydrogen, a lower alkyl having 1-4 carbons, halogen, nitro, —OR₇, —SR₇, —NR₇R₈, or —(CF)ₙCF₃, but R₅ is not hydrogen if R₆ , R₁₀, R₁₁, R₁₂ and R₁₃ are all hydrogen, Z, Z′, Z″, Z′″, and Z″″ are all carbon, and R′ and R″ represent H, OH, C₁-C₄ alkoxy or C₁-C₄ acyloxy or R′ and R″ taken together form an oxo, methano, or hydroxyimino group;

R₆, R₁₀, R₁₁, R₁₂ and R₁₃ each independently represent hydrogen, a lower alkyl having 1-4 carbons, halogen, nitro, —OR₇, —SR₇, —NR₇R₈ or —(CF)ₙCF₃, and exist only if the Z, Z′, Z″, Z′″, or Z″″ from which R₆, R₁₀, R₁₁, R₁₂ or R₁₃ originates is C, or R₆, R₁₀, R₁₁, R₁₂ and R₁₃ each independently represent hydrogen or a lower alkyl having 1-4 carbons if the Z, Z′, Z″, Z′″, or Z″″ from which R₆, R₁₀, R₁₁, R₁₂, or R₁₃ originates is N, and where one of R₆, R₁₀, R₁₁, R₁₂ or R₁₃ is X;

R₇ represents hydrogen or a lower alkyl having 1-6 carbons;

R₈ represents hydrogen or a lower alkyl having 1-6 carbons;

R₉ represents a lower alkyl having 1-4 carbons, phenyl, aromatic alkyl, or q-hydroxyphenyl, q-bromophenyl, q-chlorophenyl, q-florophenyl, or q-iodophenyl, where q=2-4;

R₁₄ represents hydrogen, a lower alkyl having 1-4 carbons, oxo, hydroxy, acyl having 1-4 carbons, halogen, thiol, or thioketone;

R₁₇ is hydrogen, lower alkyl having 1-8 carbons, alkenyl optionally substituted with halogen, acyl, —OR₇ or —SR₇, —R₉, alkyl carboxylic acid optionally substituted with halogen, acyl, —OR₇ or —SR₇ substituted, alkenyl carboxylic acid optionally substituted with halogen, acyl, —OR₇ or —SR₇, alkyl amine optionally substituted with halogen, acyl, —OR₇ or —SR₇, or alkenyl amine optionally substituted with halogen, acryl, —OR₇ or —SR₇;

R₁₈ represents hydrogen, a lower alkyl having 1-4 carbons, halogen, nitro, —OR₇, —SR₇, —NR₇R₈, or —CF)ₙCF₃;

X is —COOH, tetrazole, —PO₃H, —SO₃H, —CHO, —CH₂OH, —CONH₂, —COSH, —COOR₉, —COSR₉, —CONHR₉, or —COOW where W is a pharmaceutically acceptable salt, and wherein X can originate from any C or N on the ring;

Z, Z′, Z″, Z′″ and Z″″ each independently is C, S, O, N, or a pharmaceutically acceptable salt, provided that one or more of Z, Z′, Z″, Z′″ and Z″″ are not O or S if Z, Z′, Z″, Z′″ or Z″″ is attached by a double bond to one of Z, Z′, Z″, Z′″ or Z″″ or if one or more of Z, Z′, Z″, Z′″ or Z″″ is attached to one of Z, Z′, Z″, Z′″ or Z″″ that is O or S, and provided that one or more of Z, Z′, Z″, Z′″ and Z″″ are not N if one of Z, Z′, Z″, Z′″ and Z″″ is attached by a single bond to one of Z, Z′, Z″, Z′″ and Z″″ that is N;

n is 0 to 3; and

the dashed lines are optional double bonds.

In a particular embodiment, compounds of Structural Formula (XXI)-(XXVII) are administered to subjects having cachexia associated with one or more diseases, disorders or conditions selected from the group consisting of cancer, AIDS, liver cirrhosis, chronic renal failure, chronic obstructive pulmonary disease, chronic cardiac failure, immune system diseases, tuberculosis, cystic fibrosis, gastrointestinal disorders, an aged condition and sarcopenia.

Described below are additional groups of compounds that can be used in treating cachexia, without limitation as to the primary disease, disorder or condition with which the cachexia is associated.

A first group of compounds useful in treating cachexia is represented by Structural Formula (XXVIII):

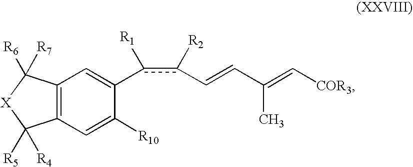

where:

the dotted bond is optional, provided that when:
a) the dotted bond is present, R₁ is lower alkyl and R₂ is halogen, or R₁ and R₂ taken together with the carbon atoms to which they are attached form a 5 to 8 membered carbocyclic ring or a 5 to 8 membered heterocyclic ring containing one sulfur, oxygen or nitrogen atom, wherein when said ring is aromatic, the dotted bond is part of a mesomeric system, and
b) the dotted bond is absent, R₁ and R₂ taken together are methylene, thereby forming a cis-substituted cyclopropyl ring;

R₃ is hydroxy or lower alkoxy;

R₄, R₅, R₆ and R₇ are, independently, hydrogen or lower alkyl;

X is (>CR₈R₉)ₙ;

n is 1, 2 or 3;

R₈ and R₉ are, independently, hydrogen or lower alkyl; and

R₁₀ is hydrogen, alkyl or alkoxy;

and pharmaceutically acceptable salts of carboxylic acids of Structural Formula (XXVIII).

A second group of compounds useful in treating cachexia is represented by Structural Formula (XXIX):

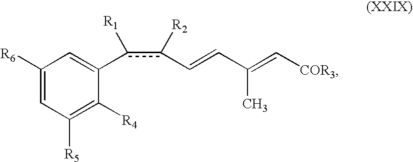

where:

the dotted bond is either hydrogenated or forms a double bond, provided that:
a) when the dotted bond forms a double bond, R₁ is lower alkyl and R₂ is hydrogen; and
b) when the dotted bond is hydrogenated, R₁ and R₂ taken together are methylene to form a cis-substituted cyclopropyl ring;

R₃ is hydroxy or lower alkoxy;

R₄ is alkyl or alkoxy; and

R₅ and R₆ are, independently, a C₄₋₁₂ alkyl or a C₅₋₁₂ cycloalkyl substituent containing from 1-3 rings which are either unsubstituted or substituted with from 1-3 lower alkyl groups, with the carbon atom of R₅ and R₆ being linked to the remainder of the molecule to form a quaternary carbon atom; or

R₅ and R₆ are independently a C₄₋₁₂ alkyl group or a mono- or polycyclic C₅₋₁₂ hydrocarbon group that are linked to the phenyl ring through a quaternary carbon atom, and pharmaceutically acceptable salts thereof.

A third group of compounds useful for treating cachexia are represented by Structural Formula (XXX):

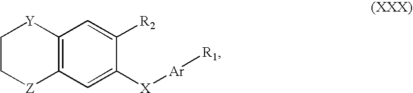

wherein:

R₁ is a hydrogen atom, a —CH₃ radical, a —CH₂OR₃ radical, a —CH₂OCOR₄ radical, an —OR₅ radical, an —O(CH₂)ₘ(CO)ₙR₆ radical, a —COR₇ radical, a —COOR₈ radical or an —S(O)ₚR₉ radical;

R₂ is a hydrogen atom or a halogen atom, a lower alkyl radical, an —NO₂ radical, an —OCOR₄ radical, an —OR₉ radical or a —NR₉R₁₀ radical;

Ar is a radical selected from among those of the following formulae (a)-(e):

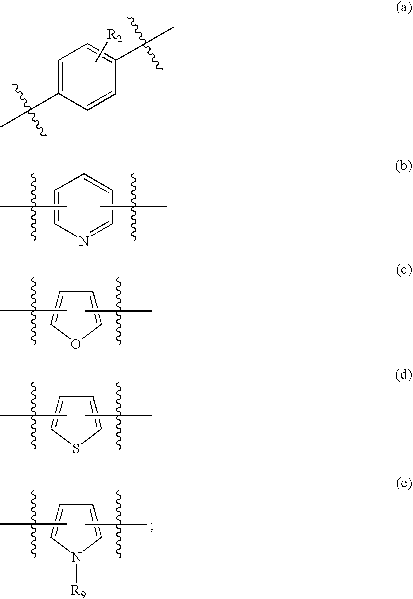

X is —O—, —S(O)ₜ— or an —NR₉— radical;

Y and Z are each —O—, —S(O)ₜ— or a radical —CR₁₁R₁₂;

m is an integer equal to 1, 2 or 3;

n is an integer equal to 0 or 1;

p is an integer equal to 0, 1, 2 or 3;

t is an integer equal to 0, 1 or 2;

R₃ is a hydrogen atom or a lower alkyl radical;

R₄ is a lower alkyl radical;

R₅ is a hydrogen atom or a lower alkyl radical;

R₆ is a lower alkyl radical or a heterocycle;

R₇ is a hydrogen atom, a lower alkyl radical or an —NR′R″ radical;

R′ and R″ are identical or different, and are each a hydrogen atom, a lower alkyl radical, a mono- or polyhydroxyalkyl radical, an optionally substituted aryl radical, or an amino acid or peptide or sugar residue, or R′ and R″ together form, with the nitrogen atom from which they depend, a nitrogen-containing heterocycle;

R₈ is a hydrogen atom, a linear or branched alkyl radical having from 1 to 20 carbon atoms, an alkenyl radical, a mono- or polyhydroxyalkyl radical, an optionally substituted aryl or aralkyl radical, or a sugar residue or an amino acid or peptide residue;

R₉ is a hydrogen atom or a lower alkyl radical;

R₁₀ is a hydrogen atom or a lower alkyl radical;

R₁₁ is a hydrogen atom or a lower alkyl radical;

R₁₂ is a hydrogen atom or a lower alkyl radical, with the proviso that Y and Z are not simultaneously each an oxygen atom or an —S(O)ₜ— radical.

A fourth group of compounds useful for treating cachexia are represented by Structural Formula (XXXI):

Z—(CR³═CR²)ₙ—COOR¹ (XXXI)

where:

R¹ is hydrogen or a carboxyl-protecting group;

R² and R³ are each independently hydrogen atom, halogen, linear lower alkyl, branched lower alkyl, linear lower alkoxy, branched lower alkoxy or aryl;

n is an integer of 1 to 3;

nR²'s or nR³'s are the same or different from one another; and

Z is a group represented by one of the following formulas:

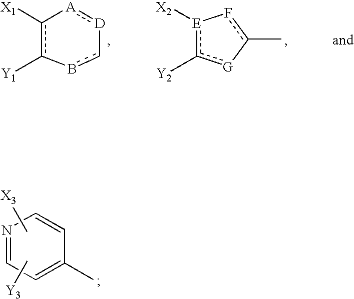

A, B and D are each carbon, nitrogen, sulfur or oxygen, where the carbon or nitrogen atoms are optionally substituted;

X₁ and Y₁ are each independently hydrogen, —NR⁴R⁵, —CR⁶R⁷R⁸, —OR⁹, —SR¹⁰, —S(O)R¹¹ or —S(O)2R12, or alternatively X₁ and Y₁ together with the carbon atoms to which they are bonded form an optionally substituted, saturated or unsaturated ring optionally containing oxygen, sulfur and/or nitrogen, and the substituents on the saturated or unsaturated ring are optionally united to form a saturated or unsaturated ring optionally containing oxygen, sulfur and/or nitrogen;

R⁴ and R⁵ are each independently hydrogen, linear lower alkyl, branched lower alkyl or cycloalkyl, or optionally when A or B is a carbon atom optionally bearing a substituent, R⁴ or R⁵ together with the substituent of A or B form a ring;

R⁶, R⁷ and R⁸ are each independently hydrogen, linear lower alkyl or branched lower alkyl; and

R⁹, R¹⁰, R¹¹ and R¹² are each independently hydrogen, linear lower alkyl or branched lower alkyl;

E is a carbon or nitrogen;

F and G are each independently carbon, nitrogen, sulfur or oxygen, where the carbon or nitrogen atoms are optionally substituted;

X₂ and Y₂ are each independently hydrogen, —NR¹³R¹⁴, —CR¹⁵R¹⁶R¹⁷, —OR¹⁸, —SR¹⁹, —S(O)R²⁰ or —S(O)₂R²¹, or alternatively X² and Y² taken together form an optionally substituted, saturated or unsaturated ring optionally containing oxygen, sulfur and/or nitrogen;

R¹³ and R¹⁴ are each independently hydrogen, linear lower alkyl, branched lower alkyl or cycloalkyl;

R¹⁵, R¹⁶ and R¹⁷ are each independently hydrogen, linear lower alkyl or branched lower alkyl;

R¹⁸, R¹⁹, R²⁰ and R²¹ are each independently hydrogen, linear lower alkyl or branched lower alkyl;

X³ and Y³ are each independently hydrogen, linear or branched lower alkyl, linear or branched lower alkoxy, cycloalkyl, aryl, heteroaryl, fluoroalkyl or halogeno; and

the symbol [symbol] represents a single bond or a double bond, with the proviso that where Z is not

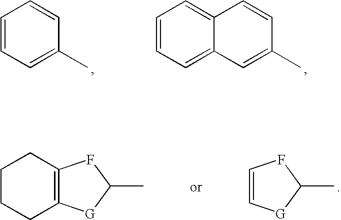

A fifth group of compounds suitable for treating cachexia are represented by Structural Formula (XXXII):

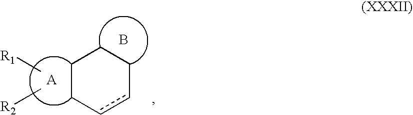

where:

R₁ and R₂ are each independently hydrogen, lower alkyl, alkenylalkyl, alkynylalkyl, cycloalkyl, cycloalkylalkyl, lower alkoxyalkyl, aryl, heteroaryl or arylalkyl, or alternatively R₁ and R₂ are united to form a 5- to 7-membered cycloalkyl group which is substituted with a lower alkyl group and optionally contains sulfur, oxygen, sulfinyl, sulfonyl or NR₃;

R₃ is hydrogen or lower alkyl;

the broken line moiety represents a single bond or a double bond;

A represents

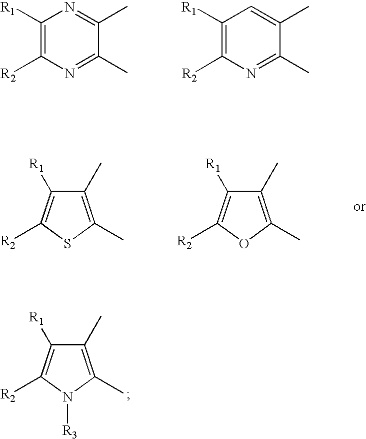

B represents

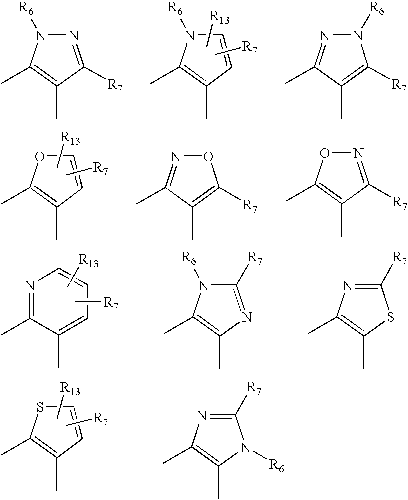

R₆ is hydrogen, lower alkyl, alkenylalkyl, alkynylalkyl, cycloalkyl, cycloalkylalkyl, lower alkoxyalkyl, aryl, heteroaryl, arylalkyl or heteroarylalkyl;

R₁₃ is hydrogen, lower alkyl or lower alkoxy;

R₇ is -E-C(═O)R₈;

E is aryl, heteroaryl or

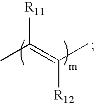

R₁₁ and R₁₂ are each hydrogen or lower alkyl;

m is an integer of 1 to 3;

R₈ is hydrogen, hydroxyl, lower alkoxy or —NR₉R₁₀; and

R₉ and R₁₀ are each independently hydrogen, hydroxyl, lower alkyl, lower alkoxy, hydroxyalkyl, aryl, hydroxyaryl or heteroaryl, or alternatively R₉ and R₁₀ together with the nitrogen atom to which they are bonded may form a ring optionally containing nitrogen, oxygen or sulfur.

Additional compounds useful for the treatment of cachexia are represented by Structural Formulas (XXXIII)-(XXXVII):

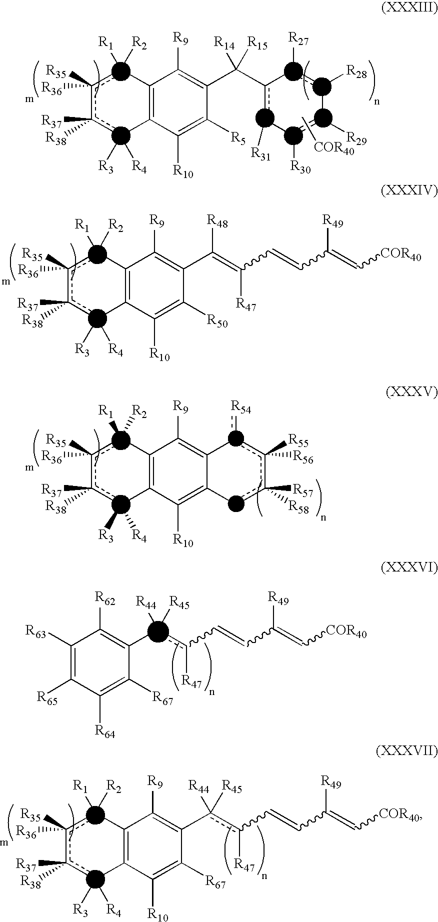

where:

R₁ through R₄ each independently are hydrogen, a C₁-C₆ alkyl or a C₇-C₁₅ arylalkyl or heteroarylalkyl;

R₅ is a C₅-C₁₀ alkyl, heteroalkyl, aryl, heteroaryl, a C₇-C₁₅ arylalkyl or heteroarylalkyl, —NR₆R₇, or —OR₈, where R₆ and R₇ each independently are a C₇-C₁₀ alkyl, heteroalkyl, a C₇-C₁₅ arylalkyl or heteroarylalkyl, a C₃-C₁₀ acyl, provided that only one of R₆ or R₇ is acyl, or R₆ and R₇ taken together are C₃-C₆ cycloalkyl, and where R₉ is a C₇-C₁₀ alkyl, heteroalkyl, aryl, heteroaryl, or a C₇-C₁₅ arylalkyl or heteroarylalkyl;

R₉ and R₁₀ each independently are hydrogen, a C₁-C₁₀ alkyl, halogen, heteroarylalkyl, —NR₁₁R₁₂, —NO₂ or —OR₁₃, where R₁₁ and R₁₂ each independently are hydrogen, a C₁-C₁₀ alkyl, heteroalkyl, a C₇-C₁₅ arylalkyl or heteroarylalkyl, a C₁-C₈ acyl, provided that only one of R₁₁ or R₁₂ is acyl, or R₁₁ and R₁₂ taken together are a C₃-C₆ cycloalkyl, and where R₁₃ is hydrogen or a C₁-C₁₀ alkyl, heteroalkyl or a C₇-C₁₅ arylalkyl or heteroarylalkyl;

R₁₄ and R₁₅ each independently are hydrogen, a C₁-C₁₀ alkyl, a C₁-C₈ acyl, or OR₁₆ where R₁₆ is hydrogen or a C₁-C₁₀ alkyl; or R₁₄ and R₁₅ taken together are keto, methano, optionally substituted oxime, optionally substituted hydrazine, optionally substituted epoxy, 1,3-dioxolane, 1,3-dioxane, 1,3-dithiolane, 1,3-dithiane, oxazolidine or:

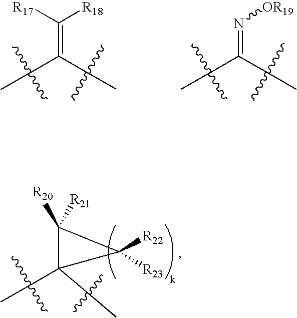

where the dashed lines crossing the bonds indicate the attachment bonds to the rings adjacent to R₁₄ and R₁₅;

R₁₇ and R₁₈ each independently are hydrogen, a C₁-C₁₀ alkyl, heteroalkyl, aryl, a C₇-C₁₅ arylalkyl or heteroarylalkyl or R₁₇ and R₁₈ taken together are a C₃-C₆ cycloalkyl;

R₁₉ is hydrogen, a C₁-C₁₀ alkyl, heteroalkyl, aryl, heteroaryl, a C₇-C₁₅ arylalkyl or heteroarylalkyl;

R₂₀ through R₂₃ each independently are hydrogen, halogen, a C₁-C₁₀ alkyl, heteroalkyl, aryl, heteroaryl, a C₇-C₁₅ arylalkyl or heteroarylalkyl, —NR₂₄R₂₅, —NO₂, or —OR₂₆, where R₂₄ and R₂₅ each independently are hydrogen, a C₁-C₁₀ alkyl, heteroalkyl, a C₇-C₁₅ arylalkyl or heteroarylalkyl or a C₁-C₈ acyl, provided that only one of R₂₄ or R₂₅ is acyl, and where R₂₆ is hydrogen or a C₁-C₁₀ alkyl, heteroalkyl, aryl, heteroaryl, or a C₇-C₁₅ arylalkyl or heteroarylalkyl;

R₂₇ through R₃₁ each independently are hydrogen, a C₁-C₁₀ alkyl, heteroalkyl, halogen, —NR₃₂R₃₃, —NO₂ or —OR₃₄, where R₃₂ and R₃₃ each independently are hydrogen, a C₁-C₁₀ alkyl, a C₇-C₁₅ arylalkyl or heteroarylalkyl, a C₁-C₈ acyl, provided that only one of R₃₂ or R₃₃ is acyl, or R₃₂ and R₃₃ taken together are a C₃-C₆ cycloalkyl, and where R₃₄ is hydrogen or a C₁-C₁₀ alkyl, heteroalkyl or a C₇-C₁₅ arylalkyl or heteroarylalkyl and exist only when W is C;

R₃₅ through R₃₈ each independently are hydrogen, a C₁-C₂ alkyl or —OR₃₉ where R₃₉ is hydrogen or a C₁-C₁₀ alkyl, or R₃₅ and R₃₆ or R₃₇ and R₃₈ taken together are keto, or R₃₅ and R₃₆, R₃₇ and R₃₈, R₃₅ and R₃₇ or R₃₆ and R₃₈ taken together are epoxy;

COR₄₀ can originate from any W when the originating W is C, and R₄₀ is —OR₄₁ or —NR₄₂R₄₃, with R₄₁ being hydrogen, a C₁-C₆ alkyl or a C₇-C₁₅ arylalkyl or heteroarylalkyl, and with R₄₂ and R₄₃ each independently being hydrogen, a C₁-C₆ alkyl, a C₇-C₁₅ arylalkyl or heteroarylalkyl, aryl, ortho-, meta, or para-substituted hydroxyarl, or taken together are a C₃-C₆ cycloalkyl;

R₄₄ and R₄₅ each independently are hydrogen, a C₁-C₄ alkyl or —CH₂OR₄₆, where R₄₆ is hydrogen or a C₁-C₆ alkyl, or R₄₄ and R₄₅ taken together are a C₃-C₆ cycloalkyl or cycloheteroalkyl;

R₄₇ is hydrogen, a C₁-C₄ alkyl, or when n=1, R₄₇ taken together with R₄₄ or R₄₅ is a C₃-C₆ cycloalkyl or cycloheteroalkyl;

R₄₈ and R₄₉ each independently are C₁-C₄ alkyl;

R₅₀ is a C₄-C₁₀ alkyl, keteroalkyl, aryl, heteroaryl, a C₇-C₁₅ arylalkyl or heteroarylalkyl, —NR₅₁R₅₂, or —OR₅₃, where R₅₁ and R₅₂ each independently are a C₂-C₁₀ alkyl, heteroalkyl, a C₇-C₁₅ arylalkyl or heteroarylalkyl, a C₃-C₁₀ acyl, provided that only one of R₅₁ or R₅₂ is acyl, or R₅₁ and R₅₂ taken together are C₃-C₆ cycloalkyl, and where R₅₃ is a C₇-C₁₀ alkyl, heteroalkyl, aryl, heteroaryl, a C₃-C₆ alkyl, heteroalkyl, aryl or heteroalkyl or a C₇-C₁₅ arylalkyl or heteroarylalkyl;

R₅₄ represents:

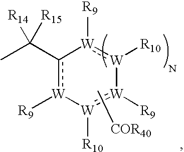

where R₉, R₁₀, R₁₄, R₁₅ and R₄₀ have the definitions given above;

R₅₅ through R₅₈ each independently are hydrogen, halogen, a C₁-C₁₀ alkyl, heteroalkyl, aryl, heteroaryl, a C₇-C₁₅ arylalkyl or heteroarylalkyl, —NR₅₉R₆₀ or —OR₆₁, where R₅₉ and R₆₀ each independently are hydrogen, a C₁-C₁₀ alkyl or heteroalkyl, a C₇-C₁₅ arylalkyl or heteroarylalkyl, a C₁-C₈ acyl, provided that only one of R₅₉ or R₆₀ is acyl, or R₅₉ and R₆₀ taken together are C₃-C₆ cycloalkyl, and where R₆₁ is hydrogen or a C₁-C₁₀ alkyl, heteroalkyl, aryl, heteroaryl, or a C₇-C₁₅ arylalkyl or heteroarylalkyl, or where R₅₅ and R₅₆ or R₅₇ and R₅₈ taken together are keto, methano, a C₁-C₁₀ alkyl methylene, a C₁-C₁₀ dialkylmethylene, C₇-C₁₅ arylalkyl or heteroarylalkylmethylene, oxime, O-alkyl oxime, hydrazone, 1,3-dioxolane, 1,3-dioxane, 1,3-dithiolane, 1,3-dithiane, oxazolidine, or R₅₅ and R₅₇ or R₅₆ and R₅₈ taken together are epoxy;

R₆₂ through R₆₄ each independently are hydrogen, aryl, heteroaryl, —CF₃, a C₂-C₆ alkyl, C₂-C₆ heteroalkyl or —NR₅₁R₅₂, where R₅₁ and R₅₂ have the definitions given above;

R₆₅ is hydrogen, a C₁-C₂ alkyl or —OR₆₆, where R₆₆ is a C₁-C₂ alkyl;

R₆₇ is a C₄-C₁₀ alkyl, heteroalkyl, aryl, heteroaryl, a C₇-C₁₅ arylalkyl or heteroarylalkyl, —NR₅₁R₅₂, or —OR₆₈, where R₅₁ and R₅₂ have the definitions described above, and where R₆₈ is a C₃-C₁₀ alkyl, heteroalkyl, aryl, heteroaryl, or a C₇-C₁₅ arylalkyl or heteroarylalkyl;

X and Y each independently represent C, O, S, N, SO or SO₂, provided, however, that when X or Y are O, S, SO or SO₂, then either R₁ and R₂ or R₃ and R₄, respectively do not exist, and further provided, that when X or Y is N, then one each of R₁ and R₂ or R₃ and R₄, respectively, does not exist;

M is N or C;

Q is N or C;

Z is O, S, SO, SO₂, CR₆₉R₇₀ or NR₇₁, where R₆₉ through R₇₁ each independently are hydrogen or a C₁-C₁₀ alkyl, heteroalkyl, aryl, heteroaryl, a C₇-C₁₅ arylalkyl or heteroarylalkyl, or R₆₉ and R₇₀ each independently are —OR₇₁, or R₆₉ and R₇₀ taken together are a cycloalkyl;

each W is independently C, N, S or O, or a pharmaceutically acceptable salt, but is not O or S if attached by a double bond to another W or if attached to another such W which is O or S, and is not N if attached by a single bond to another such W which is N;

m is 0, 1 or 2 carbon atoms;

n is 0 or 1 carbon atoms;

k is 1 to 5 carbon atoms;

the dashed lines in the structures, other than at R₁₄ and R₁₅, represent optional double bonds, provided, however, that the double bonds are not contiguous, and further provided that when such optional double bonds exist then the substitution patterns around such bonds cannot violate double bond valency; and the wavy lines represent olefin geometry that is either cis (Z) or trans (E), and unless otherwise indicated, for substituents R₁ through R₇₁, all olefin geometric isomers (i.e., cis (Z) or trans (E)) of the above compounds are included.

Yet another group of compounds suitable for treating cachexia is represented by Structural Formula (XXXVIII):

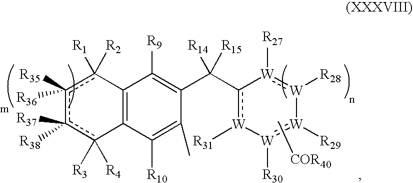

where:

all variables in the structures are as defined above for Structural Formulas (XXX)-(XXXIV), with the exception of new variable R₇₂, which is a C₃-C₁₀ alkyl, heteroalkyl, aryl, heteroaryl, a C₇-C₁₅ arylalkyl or heteroarylalkyl, NR₇₃R₇₄, or OR₇₅, where R₇₃ and R₇₄ each independently are a C₇-C₁₀ alkyl, heteroalkyl, a C₇-C₁₅ arylalkyl or heteroarylalkyl, a C₃-C₁₀ acyl, provided that only one of R₇₃ or R₇₄ is acyl, or R₇₃ and R₇₄ taken together are C₃-C₆ cycloalkyl, and where R₇₅ is a C₂-C₁₀ alkyl, heteroalkyl, aryl, heteroaryl, or a C₇-C₁₅ arylalkyl or heteroarylalkyl.

A further group of compounds useful for treating cachexia are represented by Structural Formula (XXXIX):

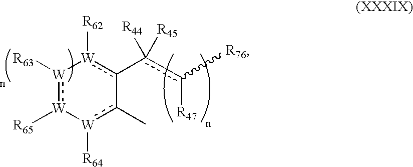

where:

R₄₄ through R₄₇ and R₆₂ through R₆₈, M, W and n each have the definitions given above for Structural Formulas (XXXIII)-(XXXVII), or R₆₂ and R₆₃, R₆₃ and R₆₅, or R₆₅ and R₆₄ taken together are:

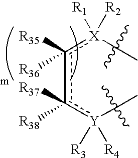

where R₁ through R₄, R₃₅ through R₃₉, X, Y and m have the definitions given above for Structural Formulas (XXXIII)-(XXXVII) and the dashed lines crossing the bonds adjacent to X and Y indicate the points of attachment at R₆₂ and R₆₃, R₆₃ and R₆₅, or R₆₅ and R₆₄;

R₇₆ is:

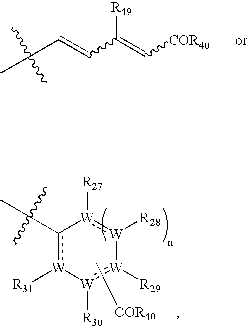

where R₂₇ through R₃₄, R₄₀ through R₄₃, R₄₉, W and n have the same definitions given above for Structural Formulas (XXXIII)-(XXXVII) and the dashed lines crossing the bonds adjacent to R₄₉ and R₂₇/R₃₁ indicate the points of attachment at R₇₆;
other than as indicated above for points of attachment, the dashed lines in the structures represent optional double bonds, provided, however, that the double bonds cannot be contiguous, and further provided that when such optional double bonds exist then the substitution patterns around such bonds cannot violate double bond valency; and the wavy lines represent olefin geometry that is either cis (Z) or trans (E), and unless otherwise indicated, for substituents R₁ through R₇₆, all olefin geometric isomers (i.e., cis (Z) or trans (E)) of the above compounds are included.

Yet another group of compounds useful in treating cachexia are represented by Structural Formulas (LX) and (LXI):

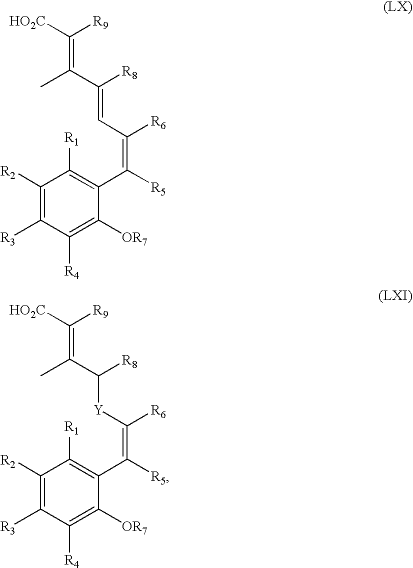

where:

R₁ is selected from the group of hydrogen, —F, —Cl, —Br, —I, C₁-C₃ alkyl, C₁-C₃ haloalkyl, C₂-C₃ alkenyl, C₂-C₃ haloalkenyl, C₂-C₃ alkynyl, C₂-C₃ haloalkynyl, and C₁-C₃ alkoxy, wherein said alkyl, haloalkyl, alkenyl, haloalkenyl, alkynyl, haloalkynyl, and alkoxy groups are optionally substituted;

R₂ and R₄ are independently selected from the group of hydrogen, —NR₁₀R₁₁, C₁-C₆ alkyl, C₁-C₆ haloalkyl, C₃-C₈ cycloalkyl, C₂-C₆ alkenyl, C₂-C₆ haloalkenyl, C₂-C₆ alkynyl, C₂-C₆ haloalkynyl, aryl, heteroaryl, C₁-C₆ alkoxy, and aryloxy, wherein said alkyl, haloalkyl, cycloalkyl, alkenyl, haloalkenyl, alkynyl, haloalkynyl, aryl, heteroaryl, alkoxy, aryloxy groups are optionally substituted;

R₃ is selected from the group of hydrogen, C₁-C₆ alkyl, C₁-C₆ haloalkyl, C₃-C₈ cycloalkyl, C₂-C₆ alkenyl, C₂-C₆ haloalkenyl, C₂-C₆ alkynyl, C₂-C₆ haloalkynyl, aryl, heteroaryl, C₁-C₆ alkoxy, and aryloxy, wherein said alkyl, haloalkyl, cycloalkyl, alkenyl, haloalkenyl, alkynyl, haloalkynyl, aryl, heteroaryl, alkoxy, aryloxy groups are optionally substituted;

R₅ and R₆ are independently selected from the group of hydrogen, —F, —Cl, —Br, —I, —CN, —NH₂, —OH, —SH, C₁-C₆ alkyl, C₁-C₆ haloalkyl, C₂-C₆ alkenyl, C₁-C₆ haloalkenyl, C₁-C₆ alkoxy, and aryloxy wherein said alkyl, haloalkyl, alkenyl, haloalkenyl, alkoxy and aryloxy groups are optionally substituted; or

R₅ and R₆ taken together form a three- to eight-membered carbocyclic ring, a three- to eight-membered heterocyclic ring, an aryl group or a heteroaryl group, wherein said carbocyclic ring, heterocyclic ring, aryl and heteroaryl groups are optionally substituted;

R₇ is selected from the group of C₂-C₆ alkyl, C₂-C₆ alkenyl, and C₂-C₆ haloalkyl, wherein said alkyl, alkenyl, and haloalkyl groups are optionally substituted;

R₈ is selected from the group of hydrogen, —F, —Cl, —Br, —I, —CN, C₁-C₆ alkyl, C₁-C₆ haloalkyl, C₂-C₆ alkenyl, C₂-C₆ haloalkenyl, C₂-C₆ alkynyl, C₁-C₆ alkoxy, and aryloxy, wherein said alkyl, haloalkyl, alkenyl, haloalkenyl, alkynyl, alkoxy, and aryloxy groups are optionally substituted;

R₉ is selected from the group of hydrogen, —F, —Cl, —Br, —I, methyl, and optionally substituted methyl;

R₁₀ and R₁₁ each independently is hydrogen or optionally substituted C₁-C₆ alkyl; or

R₁₀ and R₁₁ taken together with nitrogen form an optionally substituted five- or six-membered heterocyclic ring;

Y is selected from the group of NR₁₂, O and S; and

R₁₂ is selected from the group of hydrogen, optionally substituted C₁-C₆ alkyl, and optionally substituted C₁-C₆ haloalkyl; and pharmaceutically acceptable salts thereof.

Additional compounds suitable for treating cachexia are represented by Structural Formula (LXII), including pharmaceutically acceptable salts, solvates and hydrates thereof:

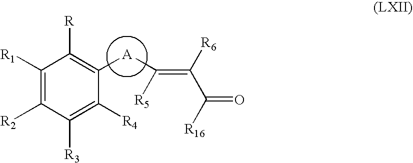

In Structural Formula (LXII), R is selected from the group of hydrogen, —F, —Cl, —Br, —I, C₁-C₃ alkyl, C₁-C₃ haloalkyl, C₂-C₃ alkenyl, C₂-C₃ haloalkenyl, C₂-C₃ alkynyl, C₂-C₃ haloalkynyl, and C₁-C₃ alkoxy, wherein said alkyl, haloalkyl, alkenyl, haloalkenyl, alkynyl, haloalkynyl, and alkoxy groups are optionally substituted;

R₁ and R₂ are each, independently, —H, a halo, a C₁-C₁₀ alkyl, a C₃-C₁₀ cycloalkyl, a C₅-C₁₀ cycloalkenyl, a 6 to 10 membered aryl, a 5 to 10 membered heteroaryl, an aryl-C₁-C₆-alkyl, or an amino group represented by the formula —NR₁₄R₁₅, wherein the alkyl, cycloalkyl, cycloalkenyl, aryl, heteroaryl and arylalkyl are optionally substituted with one or more halo, C₁-C₃ alkyl, C₁-C₃ haloalkyl or C₁-C₃ alkoxy; or R₁ and R₂ taken together with the carbon atoms to which they are attached form a five or six membered carbocyclic ring which is optionally substituted with one or more halo or C₁-C₆ alkyl groups. R₁₄ and R₁₅ are each, independently, H, a C₁-C₆ alkyl, or taken together with the nitrogen they are attached to can form a 5 to 8 heterocycle.

Alternatively, R and R₁ taken together with the carbon atoms to which they are attached form an aryl, a heteroaryl, a C₅-C₈ cycloalkyl or C₅-C₈ cycloalkenyl ring in which the aryl, heteroaryl, C₅-C₈ cycloalkyl or C₅-C₈ cyclolkenyl are optionally substituted with one or more halo, C₁-C₃ alkyl, C₁-C₃ haloalkyl or C₁-C₃ alkoxy substituents. Preferably, when R and R₁ together with the carbon atoms to which they are attached form an aryl or a heteroaryl, the aryl and heteroaryl have from five to six atoms.

R₃ is —H, a halo, a C₁-C₁₀ alkyl, a C₃-C₁₀ cycloalkyl, C₅-C₁₀ cycloalkenyl, a 6 to 10 membered aryl, a 5 to 10 membered heteroaryl, an aryl-C₁-C₆-alkyl, or an amino group represented by the formula NR₁₄R₁₅, wherein the alkyl, cycloalkyl, cycloalkenyl, aryl, heteroaryl and arylalkyl are optionally substituted with one or more halo, C₁-C₃ alkyl, C₁-C₃ haloalkyl or C₁-C₃ alkoxy.

R₄ is —H, a halo, an aryl-C₁-C₆-alkyl, a C₁-C₁₀ alkyl or a C₁-C₁₀ alkoxy group wherein the arylalkyl, alkyl, and alkoxy are optionally substituted with one or more substituents selected from halo, C₁-C₆ alkyl, aryl, heteroaryl, a C₁-C₆ alkoxy, an amino group represented by the formula —NR₁₄R₁₅. Preferably, the aryl and the heteroaryl substituents each, independently, have from five to ten atoms.

Alternatively, R₃ and R₄ taken together with the carbon atoms to which they are attached form an aryl, a heteroaryl, a C₅-C₈ cycloalkyl or a C₅-C₈ cycloalkenyl ring wherein the aryl, heteroaryl, cycloalkyl and cycloalkenyl are optionally substituted with one or more halo, C₁-C₃ alkyl, C₁-C₃ haloalkyl or C₁-C₃ alkoxy substituents. Preferably, when R₃ and R₄ together with the carbon atoms to which they are attached form an aryl or a heteroaryl, the aryl and heteroaryl have from five to ten atoms.

R₅ is —H, a halo, or a C₁-C₃ alkyl group which is optionally substituted with one or more halo.

R₆ is —H or halo.

R₁₆ is —OR₁₇, —OCH(R₁₇)OC(O)R₁₈, —NR₁₉R₂₀, or an aminoalkyl.

R₁₇, R₁₉ and R₂₀ are each, independently, —H or a C₁-C₆ alkyl.

R₁₈ is a C₁-C₆ alkyl.

Ring A is a heteroaryl group represented by the following structural formula:

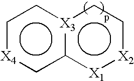

In ring A, X₁ and X₂ are each, independently, O, S, N, NH, or CH.

X₃ is N or C.

X₄ is CH or N.

p is 0 or 1.

However, when X₁ is O or S, then X₂ is CH or N and p is 0.

Ring A is optionally substituted with one or more substituents selected from a halo, a C₁-C₆ alkyl, or a C₁-C₆ alkoxy.

Additional compounds for use in treating cachexia, without limitation as to the disease, disorder or condition with which it is associated, are disclosed in the following documents: U.S. Pat. Nos. 5,770,378, 5,770,382, 5,770,383, 5,917,082, 6,048,873, 6,093,838, 6,403,638, 6,534,545 and 6,624,154; U.S. Patent Application Publication No. 20030166932; Published International Applications WO 93/21146, WO 94/12880, WO 94/17796, WO 97/12853; WO 98/22423, WO 99/06036, WO 99/58486, WO 99/58487, WO 00/020370; WO 01/070662, WO 02/071827 and WO 03/027090; and European Patent Application No. 947496, the contents of which are incorporated herein by reference. Also, the following documents disclose compounds for use in treating cachexia: V. R. Atigadda, et al. Abstracts of Papers, 226th ACS National Meeting, New York, N.Y., United States, Sep. 7-11, 2003 (2003); P. Y. Michellys, et al., Journal of Medicinal Chemistry (2003), 46(13), 2683-2696; L. J. Farmer, et al., Bioorganic & Medicinal Chemistry Letters (2003), 13(2), 261-264; B. Dominguez, et al., Bioorganic & Medicinal Chemistry Letters (2002), 12(18), 2607-2609; B. M. Forman, et al., Journal of Biological Chemistry (2002), 277(15), 12503-12506; M. I. Dawson, et al., Current Medicinal Chemistry (2002), 9(6), 623-637; V. R. Atigadda, et al., Abstracts of Papers, 223rd ACS National Meeting, Orlando, Fla., United States, Apr. 7-11, 2002 (2002); A. M. Standeven, et al., Biochemical Pharmacology (2001), 62(11), 1501-1509; M. M. Faul, et al., Abstracts of Papers, 222nd ACS National Meeting, Chicago, Ill., United States, Aug. 26-30, 2001 (2001); V. Vuligonda, et al., Journal of Medicinal Chemistry (2001), 44(14), 2298-2303; M. Ebisawa, et al., Chemical & Pharmaceutical Bulletin (2001), 49(4), 501-503; K. Ohta, et al., Chemical & Pharmaceutical Bulletin (2000), 48(10), 1504-1513; M. I. Dawson, Bioorganic & Medicinal Chemistry Letters (2000), 10(12), 1311-1313; S. S. Koch, et al., Journal of Medicinal Chemistry (1999), 42(4), 742-750; S. Hibi, et al., Journal of Medicinal Chemistry (1998), 41(17), 3245-3252; L. J. Farmer, et al., Bioorganic & Medicinal Chemistry Letters (1997), 7(21), 2747-2752; L. J. Farmer, et al., Bioorganic & Medicinal Chemistry Letters (1997), 7(18), 2393-2398; A. M. Standeven, et al., Biochemical Pharmacology (1997), 54(4), 517-524; R. L. Beard, et al., Journal of Medicinal Chemistry (1996), 39(18), 3556-3563; V. Vuligonda, et al., Bioorganic & Medicinal Chemistry Letters (1996), 6(2), 213-18; Y. Katsuta, et al., Chemical & Pharmaceutical Bulletin (1994), 42(12), 2659-61; M. F. Boehm, et al., Journal of Medicinal Chemistry (1994), 37(18), 2930-41; and M. F. Boehm, et al., Journal of Medicinal Chemistry (1995), 38(16), 3146-55, the contents of which are incorporated herein by reference.

Examples of compounds disclosed in the documents listed in the above paragraph include:

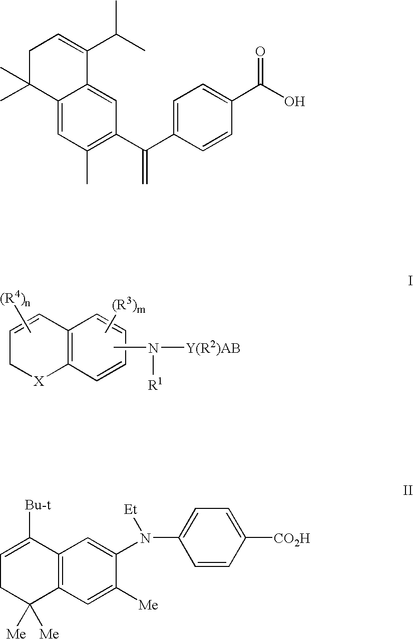

where:

X is O, S, or C(R)₂;

R is H or alkyl of 1 to 6 carbons;

R¹ is H, alkyl of 1 to 10 carbons, alkenyl of 2 to 6 carbons, phenyl-C₁-C₆ alkyl, or C₁-C₆-alkylphenyl;

R² is H, alkyl of 1 to 6 carbons, —F, —Cl, —Br, —I, —CF₃, fluoro substituted alkyl of 1 to 6 carbons, alkoxy of 1 to 6 carbons, or alkylthio of 1 to 6 carbons;

R³ is independently alkyl of 1 to 6 carbons, —F, —Cl, —Br, —I, —CF₃, fluoro substituted alkyl of 1 to 6 carbons, —OH, —SH, alkoxy of 1 to 6 carbons, fluoroalkoxy of 1 to 6 carbons, alkylthio of 1 to 6 carbons; benxyloxy, C₁-C₆ alkyl substituted benzyloxy, halogen substituted benzyloxy, phenyloxy, C₁-C₆ alkyl substituted phenyloxy, or halogen substituted phenyloxy;

R⁴ is independently —H, alkyl of 1 to 6 carbons, or —F;

Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R² groups;

m is an integer having the values 0 to 3;

n is an integer having the values 0 to 4;

A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds, and

B is hydrogen, —COOH, —COOR⁸, —CONR⁹R¹⁰, —CH₂OH, —CH₂OR¹¹, —CH₂OCOR¹¹, —CHO, —CH(OR¹²)₂, —CHOR¹³O, —COR⁷, —CR⁷(OR¹²)₂, —CR⁷OR¹³O, or tri-lower alkylsilyl, where R⁷ is an alkyl, cycloalkyl or alkenyl group containing 1 to 5 carbons, R⁸ is an alkyl group of 1 to 10 carbons or trimethylsilylalkyl where the alkyl group has 1 to 10 carbons, or a cycloalkyl group of 5 to 10 carbons, or R⁸ is phenyl or lower alkylphenyl, R⁹ and R¹⁰ independently are hydrogen, an alkyl group of 1 to 10 carbons, or a cycloalkyl group of 5-10 carbons, or phenyl or lower alkylphenyl, R¹¹ is lower alkyl, phenyl or lower alkylphenyl, R¹² is lower alkyl, and R¹³ is divalent alkyl radical of 2-5 carbons, and pharmaceutically acceptable salts.

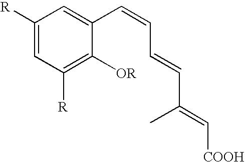

where the R groups attached directly to the phenyl ring are isopropyl or 1,1-dimethylpropyl and the R group attached to oxygen is methyl, ethyl, 2-fluoroethyl, 2,2-difluoroethyl, 2,2,2-trifluoroethyl, propyl or butyl.

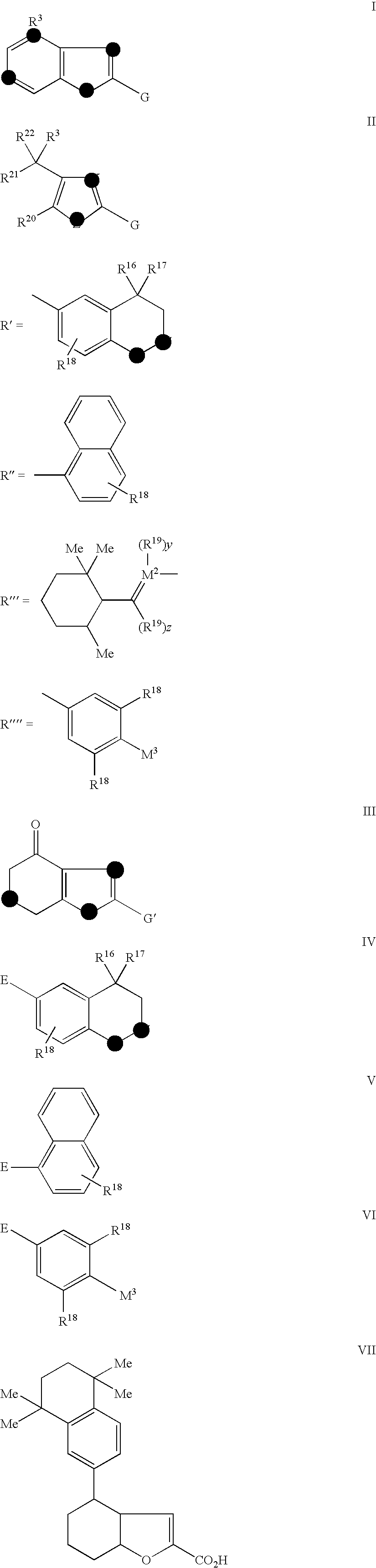

including salts, solvates, and physiologically functional derivatives thereof, where:

X is CR¹ or N, where R¹ is halogen, H, or CH₃;

Z is O, S, or NH;

M is N, C, or CR², when M is N, the ring in which M is located is non-aromatic, when M is C, the ring in which N is located is aromatic, when M is CR², then R² is H or Y(CH₂)ₙR⁶, and the A ring is non-aromatic;

Y is O or CH₂; when n is 0 to 6, R⁶ is —H, alkyl, or —CF³, but when n is 2 to 5, R⁶ is H, alkyl, —CF₃, —SO₂NR²³, —NHSO₂R²³, or —NR²³R²⁴, where R²³ is alkyl, aryl optionally substituted, heteroaryl optionally substituted or combined with R²⁴ to form a ring of 3-7 atoms; and R²⁴ is —H, alkyl, cycloalkyl or combined with R²³ to form a ring of 3 to 7 atoms;

G is —CO₂R⁷, —SOR⁷, —PO₃R⁷, —CONHOH, or

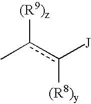

where the broken line represents an optional double bond; J is —CHO, —CO₂R⁷, —SO₃R⁷, —PO₃R⁷, —CONHOH, or J forms a thiazolidinedione ring with R⁸; R⁷ is —H or alkyl;

R⁸ and R⁹ are independently —H, halogen, alkyl, or —CF₃;

y and z are each 0, 1, or 2;

Q is CR⁴, CR⁴R⁵, O, NR, or S, where R⁴ and R⁵ are independently H or alkyl, provided that when Q is CR⁴, the A ring is aromatic;

R¹⁰ is alkyl, —COR¹¹, —CONHR¹¹, —CO₂R¹¹, —CONR¹¹R¹², —SO₂R¹¹, aryl, or cycloalkyl;

R¹¹ and R¹² are independently alkyl or cycloalkyl;

R³ is R′, wherein D is CR¹³R¹⁴, O, S, NR¹⁵, CHOH, CO, SO, SO₂, where R¹³ and R¹⁴ are independently H, alkyl, or cycloalkyl; and where R¹⁵ is H, alkyl, or cycloalkyl; D′ is (CH₂)ₘ; R¹⁶ and R¹⁷ independently are —H, C1-4 alkyl, cycloalkyl, or together form a carbocyclic ring having from 3 to 7 atoms; R¹⁸ is —H, —OR⁶, halogen, —CF₃, alkenyl, —SR¹⁶, C₁₋₄ alkyl, —CO₂R¹⁶, —COR¹¹, or —NR¹⁶R¹⁷, where R¹⁶ and R¹⁷ are as above defined; m is 0 or 1; or

R3 is R″, where R¹⁸ is as defined above; or

R³ is R′″, where M² is C or N, provided however that the optional double bond represented by the broken line is optionally present only when M² is C; each R¹⁹ is, independently, H or alkyl; y and z are as defined above; or

R³ is R″″, where each R¹⁸ is, independently, as defined above; and M³ is C(R¹⁶)₃ or N(R¹⁶)₂, when M³ is N(R¹⁶)₂, an R¹⁶ may combine with an R¹⁸ to form a 5- or 6-membered ring; and

G′ and E react to form a bond.

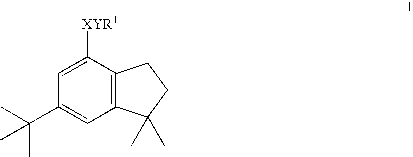

in which:

X represents:
(i) either a divalent radical of following formula:

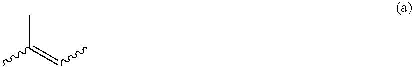

and Y then represents a divalent radical of following formula:

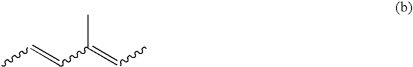

(ii) or a divalent radical of formula:

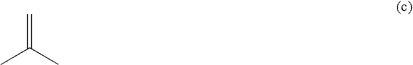

and Y then represents either a divalent radical corresponding to the divalent radical of formula (b) above or one of the divalent radicals of following formula:

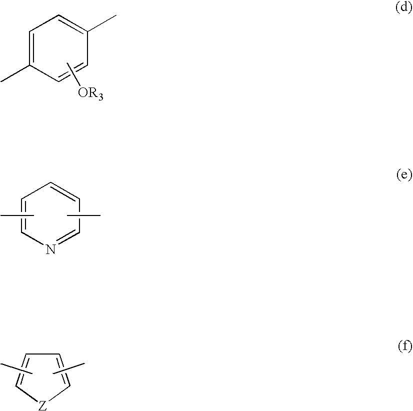

Z being —O—, —S— or >N—R₃;

R₁ represents —CH₃, —(CH₂)ₚ—OR₄, —(CH₂)ₚ—COR₅ or —S(O)ₜ—R₆, p being 0, 1, 2 or 3, t being 0, 1 or 2,

R₂ represents H or lower alkyl,

R₃ represents H, lower alkoxy or —OCOR₇,

R₄ represents H, lower alkyl, —COR₇, aryl, aralkyl, mono- or polyhydroxyalkyl, or a polyether radical,

R₅ represents H, lower alkyl, —OR₈ or —Nr′r″,

R₆ represents H or lower alkyl,

R₇ represents lower alkyl,

R₈ represents H, alkyl, alkenyl, alkynyl, aryl, aralkyl, mono- or polyhydroxyalkyl, a sugar residue or an amino acid residue,

r′ and r″, identical or different, represent H, lower alkyl, —COR₇, aryl, a sugar residue or an amino acid residue or r′ and r″, taken together, form a heterocycle, and the salts of the compounds of formula (I), when R₁ represents a carboxylic acid group, and the geometrical and optical isomers of the compounds of formula (I).

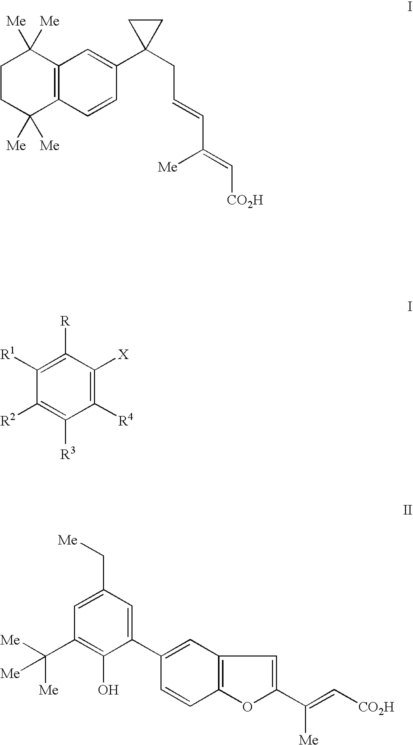

where the left hand compound corresponds to Structural Formula (LXII) above

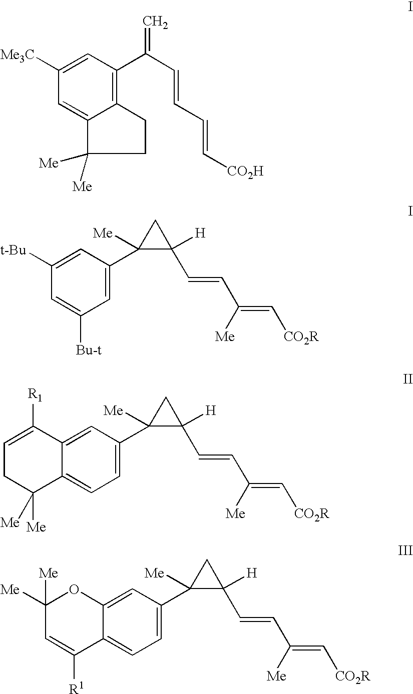

where R is —H, a salt of the carboxylic acid or lower alkyl; and R¹ is methyl, ethyl or n-propyl

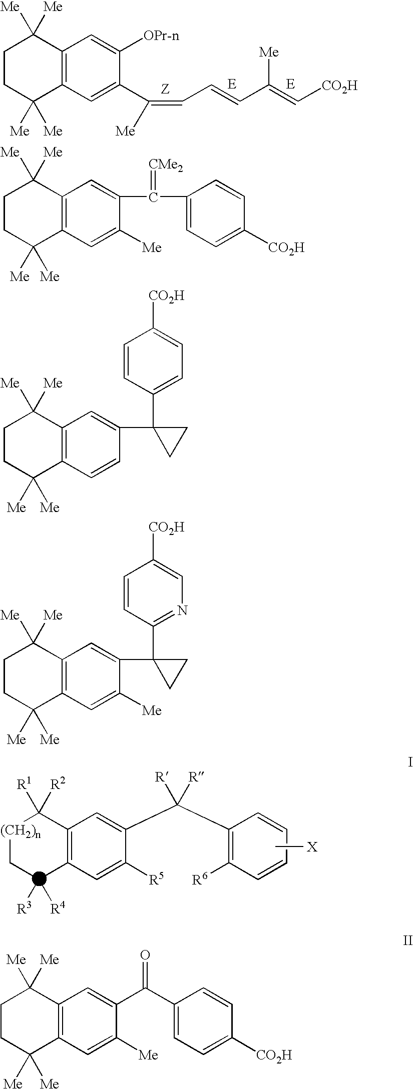

where:

R¹ and R², each independently, represent hydrogen or lower alkyl or acyl having 1-4 carbon atoms;

Y represents C, O, S, N, CHOH, CO, SO, SO₂, or a pharmaceutically acceptable salt;

R³ represents hydrogen or lower alkyl having 1-4 carbon atoms where Y is C or N;

R⁴ represents hydrogen or lower alkyl having 1-4 carbon atoms where Y is C, but R⁴ does not exist if Y is N, and neither R³ or R⁴ exist if Y is S, O, CHOH, CO, SO, or SO₂;

R′ and R″ represent hydrogen, lower alkyl or acyl having 1-4 carbon atoms, OH, alkoxy having 1-4 carbon atoms, thiol or thio ether, or amino,

or R′ or R″ taken together form an oxo (keto), methano, thioketo, HO—N═, NC—N═, (R₇R⁸)N—N═, epoxy, cyclopropyl, or cycloalkyl group and wherein the epoxy, cyclopropyl, and cycloalkyl groups can be substituted with lower alkyl having 1-4 carbons or halogen;

R⁵ represents hydrogen, a lower alkyl having 1-4 carbons, halogen, nitro, —OR⁷, —SR⁷, —NR⁷R⁸, or —(CF)ₙCF₃;

R⁶ represents hydrogen, a lower alkyl having 1-4 carbons, halogen, nitro, —OR⁷, —SR⁷, —NR⁷R⁸ or —(CF)ₙCF₃;

R⁷ represents hydrogen or a lower alkyl having 1-6 carbons;

R⁸ represents hydrogen or a lower alkyl having 1-6 carbons;

X is —COOH, tetrazole, —PO₃H, —SO₃H, —CHO, —CH₂OH, —CONH₂, —COSH, —COOR⁹, —COSR⁹, —CONHR⁹, or —COOW where R⁹ represents a lower alkyl having 1-4 carbons, phenyl, aromatic alkyl, or q-hydroxyphenyl, q-bromophenyl, q-chlorophenyl, q-fluorophenyl, or q-iodophenyl, where q=2-4, where W is a pharmaceutically acceptable salt; and

n=0-3.

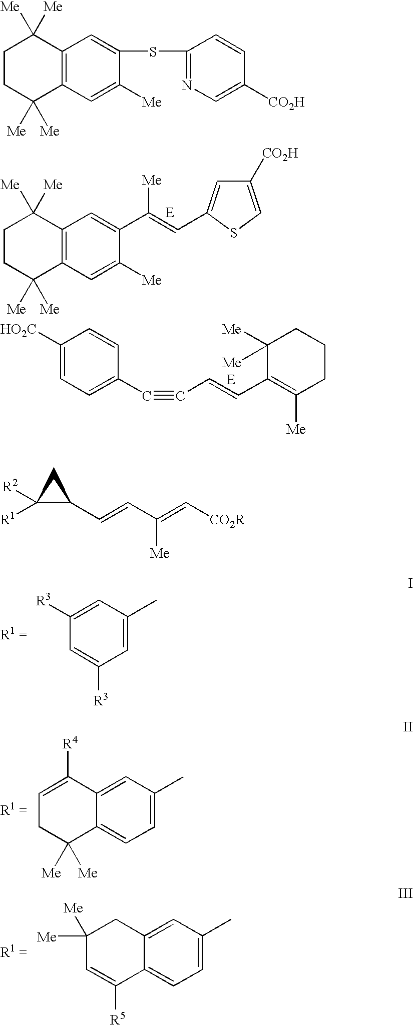

where:

R is —H, a carboxylic acid salt or lower alkyl;

R² is methyl, ethyl or propyl;

R³ is methyl, ethyl or propyl;

R⁴ is lower alkyl; and

R⁵ is lower alkyl.

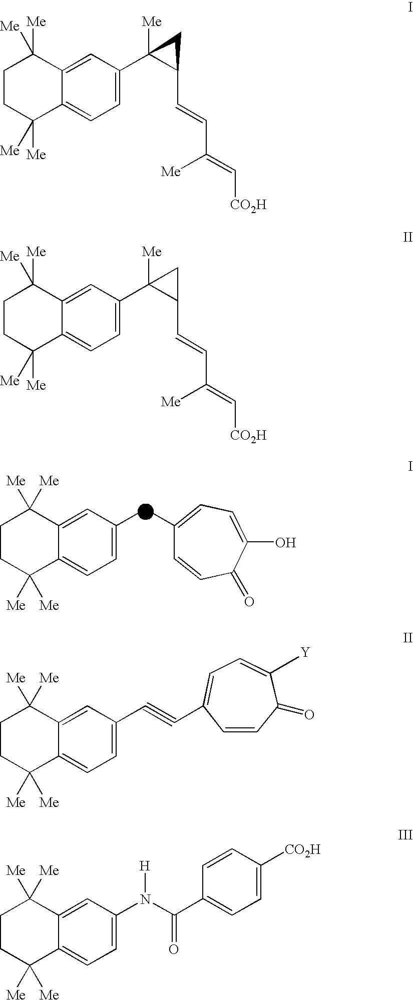

where Y is —OH, —OCH₃, —NHNH₂ or —H and Z is —C(O)NH—, —NHC(O)NH— or —N═N—.

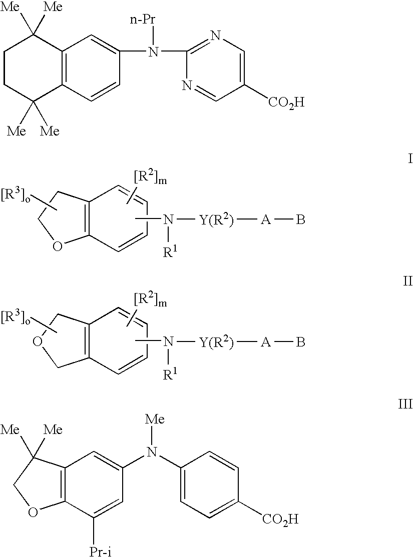

where:

R¹ is H, alkyl of 1 to 10 carbons, phenyl, heteroaryl, phenyl-C₁-C₆ alkyl, C₁-C₆-alkylphenyl, heteroaryl-C₁-C₆ alkyl, C₁-C₆-alkylheteroaryl where heteroaryl is selected from the group consisting of pyridyl, thienyl, furyl, pyridazinyl, pyrimidinyl, pyrazinyl, thiazolyl, oxazolyl, imidazolyl and pyrrazolyl;

R² is independently H, alkyl of 1 to 6 carbons, —F, —Cl, —Br, —I, —CF₃, fluoro substituted alkyl of 1 to 6 carbons, —OH, —SH, alkoxy of 1 to 6 carbons, or alkylthio of 1 to 6 carbons;

m is an integer having the values of 0 to 3;

R³ is independently —H, alkyl of 1 to 6 carbons, or —F;

o is in an integer having the values of 0 to 4;

Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups;

A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;

B is hydrogen, —COOH, —COOR⁸, —CONR⁹R¹⁰, —CH₂OH, —CH₂ OR¹¹, —CH₂OCOR¹¹, —CHO, —CH(OR¹²)₂, —CHOR¹³O, —COR⁷, —CR⁷(OR¹²)₂, —CR⁷OR¹³O, tri-lower alkylsilyl, —OH, —OR⁸ or —OCOR⁸ where R⁷ is an alkyl, cycloalkyl or alkenyl group containing 1 to 5 carbons, R⁸ is an alkyl group of 1 to 10 carbons or trimethylsilylalkyl where the alkyl group has 1 to 10 carbons, or a cycloalkyl group of 5 to 10 carbons, or R⁸ is phenyl or lower alkylphenyl, R⁹ and R¹⁰ independently are hydrogen, an alkyl group of 1 to 10 carbons, or a cycloalkyl group of 5-10 carbons, or phenyl or lower alkylphenyl, R¹¹ is lower alkyl, phenyl or lower alkylphenyl, R¹² is lower alkyl, and R¹³ is divalent alkyl radical of 2-5 carbons, and pharmaceutically acceptable salts thereof.

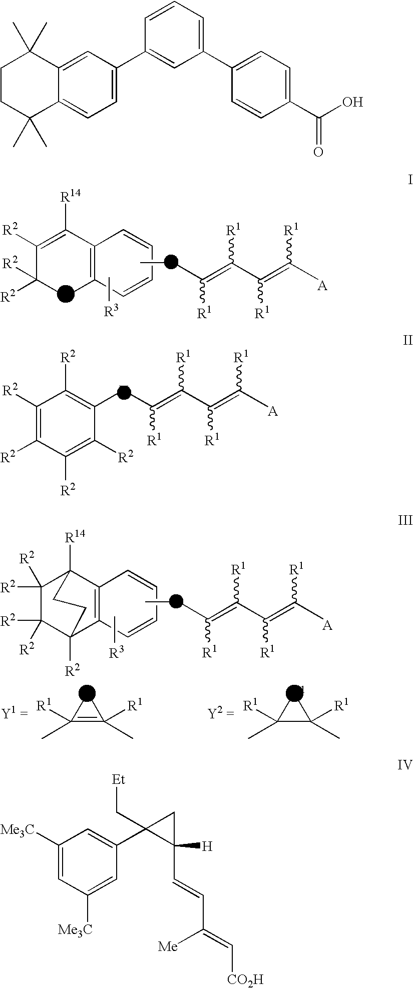

where:

X is O, S, or (CR¹R¹)ₙ where n is 0, 1 or 2;

Y is Y¹ or Y² where Z is (CR¹R¹), and o is an integer from 1 to 4, or Y is a bivalent aryl or 5 or 6 membered heteroaryl radical having 1 to 3 heteroatoms selected from N, S and O, said aryl or heteroaryl groups being unsubstituted, or substituted with 1 to 3 C₁₋₆ alkyl or with 1 to 3 C₁₋₆ fluoroalkyl groups;

X is O, S or NH;

R¹ is independently —H, lower alkyl of 1 to 6 carbons, or lower fluoroalkyl of 1 to 6 carbons;

R² is independently —H, lower alkyl of 1 to 6 carbons, OR¹, 1-adamantyl, or lower fluoroalkyl of 1 to 6 carbons, or the two R² groups jointly represent an oxo (═O) group;

R³ is hydrogen, lower alkyl of 1 to 6 carbons, OR¹, fluoro substituted lower alkyl of 1 to 6 carbons or halogen, —NO₂, —NH₂, —NHCO(C₁-C₆) alkyl, or —NHCO(C₁-C₆) alkenyl;

A is hydrogen, —COOH or a pharmaceutically acceptable salt thereof, —COOR⁸, —CONR⁹R¹⁰, —CH2OH, —CH₂OR¹¹, —CH₂OCOR¹¹, —CHO, —CH(OR¹²)₂, —CH(OR¹³O), —COR⁷, —CR⁷(OR¹²)₂, —CR⁷(OR¹³O), or —Si(C₁₋₆alkyl)₃, where R⁷ is an alkyl, cycloalkyl or alkenyl group containing 1 to 5 carbons, R⁸ is an alkyl group of 1 to 10 carbons or (trimethylsilyl) alkyl where the alkyl group has 1 to 10 carbons, or a cycloalkyl group of 5 to 10 carbons, or R⁸ is phenyl or lower alkylphenyl, R⁹ and R¹⁰ independently are hydrogen, an alkyl group of 1 to 10 carbons, or a cycloalkyl group of 5-10 carbons, or phenyl, hydroxyphenyl or lower alkylphenyl, R¹¹ is lower alkyl, phenyl or lower alkylphenyl, R¹² is lower alkyl, and R¹³ is divalent alkyl radical of 2-5 carbons, and R¹⁴ is alkyl of 1 to 10 carbons, fluoro-substituted alkyl of 1 to 10 carbons, alkenyl of 2 to 10 carbons and having 1 to 3 double bonds, alkynyl having 2 to 10 carbons and 1 to 3 triple bonds, carbocyclic aryl selected from the group consisting of phenyl, C₁-C₁₀-alkylphenyl, naphthyl, C₁-C₁₀-alkylnaphthyl, phenyl-C₁-C₁₀alkyl, naphthyl-C₁-C₁₀alkyl, C₁-C₁₀-alkenylphenyl having 1 to 3 double bonds, C₁-C₁₀-alkynylphenyl having 1 to 3 triple bonds, phenyl-C₁-C₁₀-alkenyl having 1 to 3 double bonds, phenyl-C₁-C₁₀-alkynyl having 1 to 3 triple bonds, hydroxy alkyl of 1 to 10 carbons, hydroxyalkenyl having 2 to 10 carbons and 1 to 3 double bonds, hydroxyalkynyl having 2 to 10 carbons and 1 to 3 triple bonds, acyloxyalkyl of 1 to 10 carbons, acyloxyalkenyl having 2 to 10 carbons and 1 to 3 double bonds, or acyloxyalkynyl of 2 to 10 carbons and 1 to 3 triple bonds where the acyl group is represented by COR⁸, or R¹⁴ is a 5 or 6 membered heteroaryl group having 1 to 3 heteroatoms, said heteroatoms being selected from a group consisting of O, S, and N, said heteroaryl group being unsubstituted or substituted with a C₁ to C₁₀ alkyl group, with a C₁ to C₁₀ fluoroalkyl group, or with halogen, and the dashed line in Y¹ represents a bond or absence of a bond.

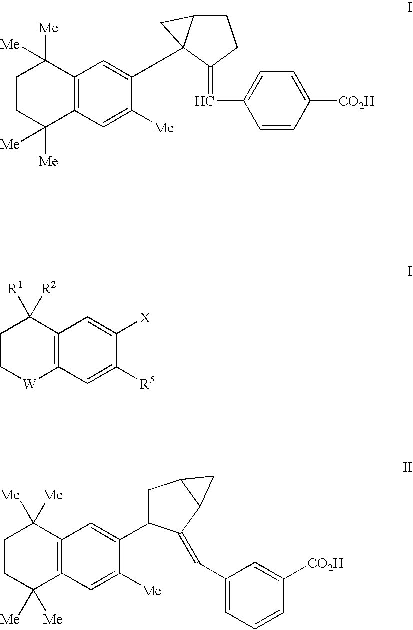

where:

R¹ and R² are independently hydrogen or C₁₋₆ alkyl;

W is C(R³)R⁴, O, NR³, S, SO or SO₂ wherein R³ and R⁴ are independently hydrogen or C₁₋₆ alkyl;

R₅ is hydrogen, C₁₋₆ alkyl, halogen, —OR¹¹, —SR¹¹, —OCOR¹¹, —NH₂, —NHR¹¹, —NR¹¹R¹², —NHCOR¹¹, —NR¹¹—COR¹² where R¹¹ and R¹² are independently C₁₋₆ alkyl, phenyl or alkyl phenyl;

X is

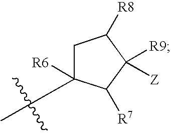

R⁶ is hydrogen, or taken together with R⁷ forms a double bond, or taken together with R⁷ is methylene to form a cyclopropyl ring;

R⁷ is hydrogen, or taken together with R⁶ forms a double bond, or taken together with R⁶ is methylene to form a cyclopropyl ring, or taken together with R⁹ forms a double bond, or taken together with R⁹ is methylene to form a cyclopropyl ring;

R⁸ is hydrogen, or taken together with R⁹ forms a double bond, or taken together with R⁹ is methylene to form a cyclopropyl ring;

R⁹ is hydrogen, hydroxy, —OR¹³, —OCOR¹³, or taken together with R⁷ forms a double bond, or taken together with R⁷ is methylene to form a cyclopropyl ring, or taken together with R⁸ forms a double bond, or taken together with R⁸ is methylene to form a cyclopropyl ring, where R¹³ is C₁₋₆ alkyl, phenyl or alkyl phenyl;

Z is —X—Y—R¹⁰, wherein X is a valence bond, phenyl or pyridyl, optionally substituted with C₁₋₃ alkyl, halogen, hydroxy, C₁₋₃ alkoxy, C₁₋₃ acyloxy, C₁₋₃ alkyl halide, thiol, C₁₋₃ substituted thiol, Y is C₁₋₆-alkyl, C₂₋₆ alkenyl or C₂₋₆ alkynyl and R¹⁰ is —CO₂H, tetrazole, —PO₃H, —SO₃H, —CO₂R¹⁵, —CONR¹⁶R¹⁷, —CH₂OH, —CHO, —CH₂OR¹⁸, —CH(OR¹⁹)₂, —HC(OR²⁰O), —COR²¹, —CR²⁰(OR¹⁹)₂, —CR²¹(OR²⁰O), wherein R¹⁵ is C₁₋₆ alkyl, phenyl or alkyl phenyl; or

Z is ═Y—R¹⁰, wherein Y is —CR¹⁴, —CR¹⁴—C₁₋₆ alkyl, —CR¹⁴phenyl, —CR¹⁴pyridyl, —CR¹⁴C₁₃alkylaryl, —CR¹⁴—C₂₋₅ alkenyl or —CR¹⁴—C₂₋₅ alkynyl, wherein R¹⁴ is H or C₁₋₃ alkyl and R¹⁰ is —CO₂H, tetrazole, —PO₃H, —SO₃H, —CO₂R¹⁵, —CONR¹⁶R¹⁷, —CH₂OH, —CHO, —CH₂OR¹⁸, —CH(OR¹⁹)₂, —HC(OR²⁰O), —COR²¹, —CR²⁰(OR¹⁹)₂, —CR²¹(OR²⁰O), wherein R¹⁵ is C₁₋₆ alkyl, phenyl or alkyl phenyl;

R¹⁶ and R¹⁷ are independently hydrogen, C₁₋₆-alkyl, C₅₋₈ cycloalkyl, phenyl or C₁₋₆-alkyl phenyl; R¹⁸ is C₁₋₆-alkyl, phenyl or C₁₋₆-alkyl phenyl; R¹⁹ is C₁₋₆ alkyl; R²⁰ is C₂₋₄ alkyl; R²¹ is C₁₋₆ alkyl phenyl or C₃₋₆ cycloalkyl;

and salts thereof with a pharmaceutically acceptable acid or base, or any optical isomer or mixture of optical isomers, including a racemic mixture, or any tautomeric forms.

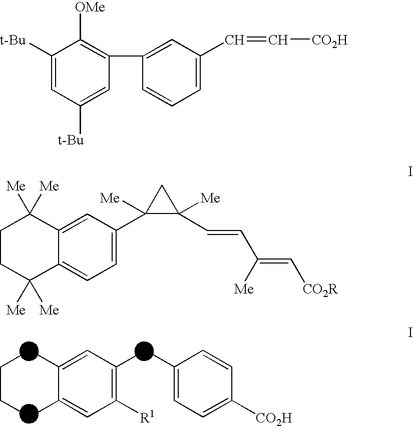

where:

Z is —C(═Q)— or

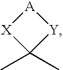

in which Q, X and Y are each independently O, S or CH₂;

A is —(CR₂)ₙ— where n is an integer of from 1 to 3;

T and T¹ are each independently O, S, CH₂, or C(CH₃)₂; and

R¹ is hydrogen or C₁-C₆ alkyl,

and pharmaceutically acceptable salts thereof.

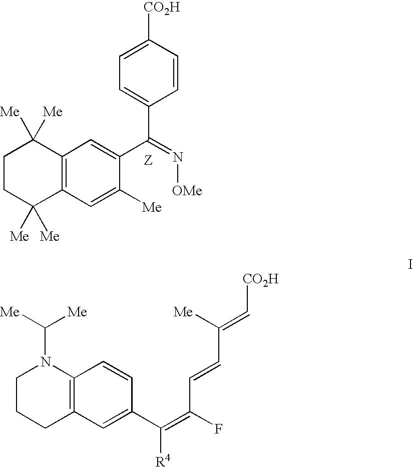

where R⁴ is methyl, ethyl, n-propyl or n-butyl.

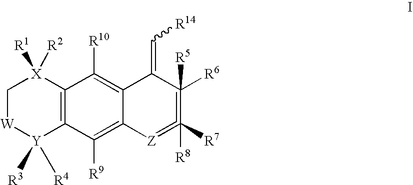

where:

R¹ through R⁴ each independently are hydrogen, a C₁-C₆ alkyl, or a C₇-C₁₅ arylalkyl;

R⁵ through R⁸ each independently are hydrogen, a C₁-C₆ alkyl, or at least two of R⁵ through R⁸ taken together are a C₃-C₆ cycloalkyl;

R⁹ and R¹⁰ each independently are hydrogen, a C₁-C₆ alkyl, —F, —Cl, —Br, —NR¹¹R¹², —NO₂ or —OR¹³, where R¹¹ and R¹² each independently are hydrogen, a C₁-C₈ alkyl, a C₇-C₁₅ arylalkyl, a C₁-C₈ acyl, provided that only one R¹¹ or R¹² can be acyl, or R¹¹ and R¹² taken together are a C₃-C₆ cycloalkyl, and where R¹³ is hydrogen or a C₁-C₈ alkyl or a C₇-C₁₅ arylalkyl;

R¹⁴ represents:

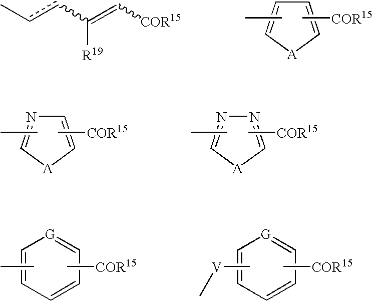

where R¹⁵ is —OR¹⁶ or —NR¹⁷R¹⁸, with R¹⁶ being hydrogen, a C₁-C₆ alkyl or a C₇-C₁₅ arylalkyl, and with R¹⁷ and R¹⁸ each independently being hydrogen, a C₁-C₆ alkyl, a C₇-C₁₅ arylalkyl, aryl, ortho-, meta-, or para-substituted hydroxyaryl, or taken together are a C₃-C₆ cycloalkyl, provided that R¹⁸ must be hydrogen when R¹⁷ is aryl or hydroxyaryl, R¹⁹ is a C₁-C₅ alkyl, and A is O, S or NR²⁰, where R²⁰ is a hydrogen, C₁-C₆ alkyl or a C₇-C₁₅ arylalky;

W is (CH₂)ₘ;

X and Y each independently represent C, O, S, N, SO or SO₂, provided, however, that when X or Y are O, S, SO or SO₂, then either R¹ and R² or R³ and R⁴ respectively do not exist, and further provided, that when X or Y is N, then one each of R¹ and R² or R³ and R⁴ respectively, do not exist;

Z is O, S, CR²²R²³ or NR²⁴, where R²² through R²⁴ each independently are hydrogen or a C₁-C₆ alkyl or R²² and R²³ taken together are a C₃-C₆ cycloalkyl;

V is C or N, provided, however, that when V is N, then no double bond exists adjacent to V;

G is C or N, provided G cannot be C when W is C;

m is 0 or 1 carbon atoms; and

n is 0, 1 or 2 carbon atoms;

the dashed lines in the structures represent optional double bonds, provided, however, that the double bonds cannot be contiguous, and further provided that when such optional double bonds exist then one each of R⁵ and R⁶ or R⁷ and R⁸ respectively do not exist; and the wavy lines represent olefin bonds that are either in the cis (Z) or trans (E) configuration.

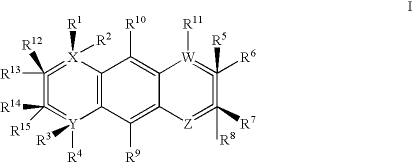

where:

R¹ through R⁴ each independently are hydrogen, a C₁-C₆ alkyl, or a C₇-C₁₅ arylalkyl;

R⁵ through R⁸ each independently are hydrogen, a C₁-C₆ alkyl, or at least two of R⁵ through R⁸ taken together are a C₃-C₆ cycloalkyl;

R⁹ and R¹⁰ each independently are hydrogen, a C₁-C₆ alkyl, —F, —Cl, —Br, —NR¹¹R¹², —NO₂ or —OR¹³, where R¹¹ and R¹² each independently are hydrogen, a C₁-C₈ alkyl, a C₇-C₁₅ arylalkyl, a C₁-C₈ acyl, provided that only one R¹¹ or R¹² can be acyl, or R¹¹ and R¹² taken together are a C₃-C₆ cycloalkyl, and where R¹³ is hydrogen or a C₁-C₈ alkyl or a C₇-C₁₅ arylalkyl;

R¹¹ represents:

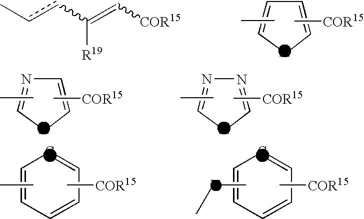

where R¹⁵ is —OR¹⁶ or —NR¹⁷R¹⁸, with R¹⁶ being hydrogen, a C₁-C₆ alkyl or a C₇-C₁₅ arylalkyl, and with R¹⁷ and R¹⁸ each independently being hydrogen, a C₁-C₆ alkyl, a C₇-C₁₅ arylalkyl, aryl, ortho-, meta-, or para-substituted hydroxyaryl, or taken together are a C₃-C₆ cycloalkyl, provided that R¹⁸ must be hydrogen when R¹⁷ is aryl or hydroxyaryl, R¹⁹ is a C₁-C₅ alkyl, and A is O, S or NR²⁰, where R²⁰ is a hydrogen, C₁-C₆ alkyl or a C₇-C₁₅ arylalky;

R¹² through R¹⁵ attached to the tricyclic ring each independently are hydrogen or a C₁-C₆ alkyl, or taken together then one each of R¹² and R¹³ or R¹⁴ and R¹⁵ respectively, form a carbonyl group;

X and Y each independently represent C, O, S, N, SO or SO₂, provided, however, that when X or Y are O, S, SO or SO₂, then either R¹ and R² or R³ and R⁴ respectively do not exist, and further provided, that when X or Y is N, then one each of R¹ and R² or R³ and R⁴ respectively, do not exist;

Z is O, S, CR²²R²³ or NR²⁴, where R²² through R²⁴ each independently are hydrogen or a C₁-C₆ alkyl or R²² and R²³ taken together are a C₃-C₆ cycloalkyl;

W is N or CR₂₅, where R₂₅ is hydrogen or a C₁-C₆ alkyl;

V is C or N, provided, however, that when V is N, then no double bond exists adjacent to V; and

G is C or N, provided G cannot be C when W is C;

the dashed lines in the structures represent optional double bonds, provided, however, that the double bonds cannot be contiguous, and further provided that when such optional double bonds exist then one each of R⁵ and R⁶ or R⁷ and R⁸ respectively do not exist; and the wavy lines represent olefin bonds that are either in the cis (Z) or trans (E) configuration.

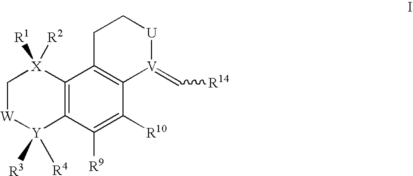

where:

R¹ through R⁴ each independently are hydrogen, a C₁-C₆ alkyl, or a C₇-C₁₅ arylalkyl;

R⁹ and R¹⁰ each independently are hydrogen, a C₁-C₆ alkyl, —F, —Cl, —Br, —NR¹¹R¹³, —NO₂ or —OR¹³, where R¹¹ and R¹² each independently are hydrogen, a C₁-C₈ alkyl, a C₇-C₁₅ arylalkyl, a C₁-C₈ acyl, provided that only one R¹¹ or R¹² can be acyl, or R¹¹ and R¹² taken together are a C₃-C₆ cycloalkyl, and where R¹³ is hydrogen or a C₁-C₈ alkyl or a C₇-C₁₅ arylalkyl;

R¹⁴ represents:

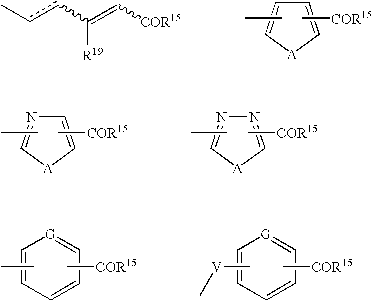

where R¹⁵ is —OR¹⁶ or —NR¹⁷R¹⁸, with R¹⁶ being hydrogen, a C₁-C₆ alkyl or a C₇-C₁₅ arylalkyl, and with R¹⁷ and R¹⁸ each independently being hydrogen, a C₁-C₆ alkyl, a C₇-C₁₅ arylalkyl, aryl, ortho-, meta-, or para-substituted hydroxyaryl, or taken together are a C₃-C₆ cycloalkyl, provided that R¹⁸ must be hydrogen when R¹⁷ is aryl or hydroxyaryl, R¹⁹ is a C₁-C₅ alkyl, and A is O, S or NR²⁰, where R²⁰ is a hydrogen, C₁-C₆ alkyl or a C₇-C₁₅ arylalky;

X and Y each independently represent C, O, S, N, SO or SO₂, provided, however, that when X or Y are O, S, SO or SO₂, then either R¹ and R² or R³ and R⁴ respectively do not exist, and further provided, that when X or Y is N, then one each of R¹ and R² or R³ and R⁴ respectively, do not exist;

U is (CH₂)ₙ where n is 0, 1 or 2 carbon atoms;

V is C or N, provided, however, that when V is N, then no double bond exists adjacent to V;

W is (CH₂)ₘ where m is 0 or 1 carbon atoms G is C or N, provided G cannot be C when W is C;

the dashed lines in the structures represent optional double bonds, provided, however, that the double bonds cannot be contiguous, and further provided that when such optional double bonds exist then one each of R⁵ and R⁶or R⁷ and R⁸ respectively do not exist; and the wavy lines represent olefin bonds that are either in the cis (Z) or trans (E) configuration.

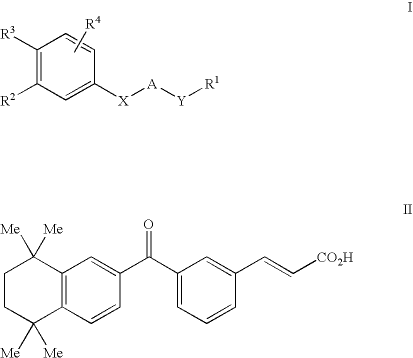

where:

R¹ represents:
(i) the radical —CH₃,
(ii) the radical —CH₂—O—R⁵,
(iii) the radical —O—R⁵,
(iv) the radical —CO—R⁶,

### R⁵ and R⁶ having the meanings given below,

Y represents a radical chosen from the radicals of formulae (a) and (b) below:

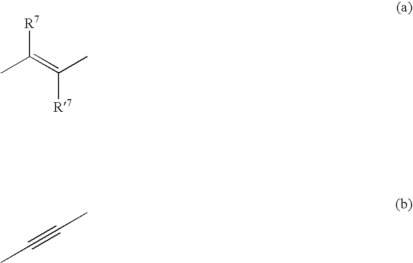

### R⁷ and R′⁷ having the meanings given below,

Ar represents a radical chosen from the radicals of formulae (c) to (f) below:

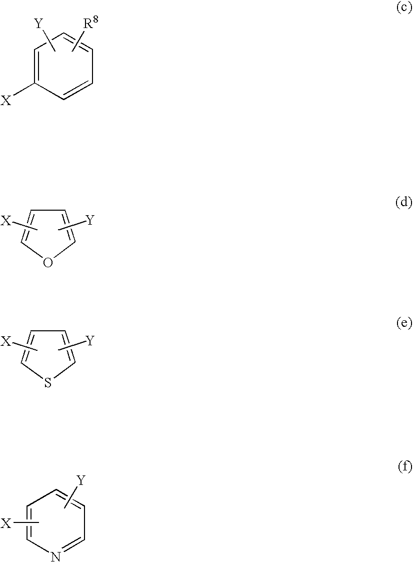

in which the radical Y is in an ortho or meta position relative to the radical X, X and Y of these formulae corresponding to X and Y represented in formula (I), R⁸ having the meaning given below,

X represents an oxygen or sulphur atom, a radical —SO—, —SO₂—, —N(R⁹)— or a radical chosen from the radicals of formulae (g) to (r) below:

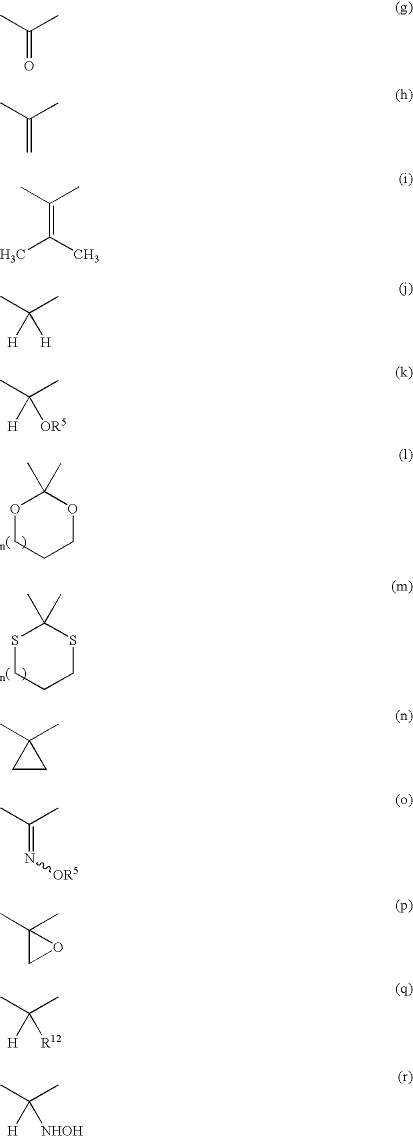

### R⁵, R⁹, R¹² and n having the meanings given below,

R² and R³, which may be identical or different, are chosen from the group consisting of:
(i) a hydrogen atom,
(ii) an alkyl radical having at least 3 carbon atoms, among which the carbon attached to the phenyl radical of formula (I) is substituted with at least two carbon atoms,
(iii) a linear or branched alkyl radical,
(iv) a radical —OR⁵,
(v) a radical —SR⁵,
(vi) a polyether radical,
   R⁵ having the meaning given below, it being understood that R² and R³, taken together, can form, with the adjacent aromatic ring, a 5- or 6-membered ring, optionally substituted with methyl groups and/or optionally interrupted by an oxygen or sulphur atom, it being understood that, when R² and R³ do not form a ring, at least one of the radicals R₂ and R³ has a meaning (ii) mentioned above,

R⁴ and R⁸, which may be identical or different, represent a hydrogen atom, a halogen atom, a linear or branched alkyl radical, or a radical —OR⁵, a polyether radical,

R⁵ represents a hydrogen atom, a lower alkyl radical or a radical —COR¹⁰, R¹⁰ having the meaning given below,

R⁶ represents:
(a) a hydrogen atom
(b) a lower alkyl radical
(c) a radical of formula —NR′R″, R′ and R″ having the meanings given below,
(d) a radical —OR¹¹, R¹¹ having the meaning given below,

R⁷, R′⁷ and R⁹, which may be identical or different, represent a hydrogen atom or a lower alkyl radical,

n is an integer equal to 0 or 1,

R¹⁰ represents a lower alkyl radical,

R¹¹ represents a hydrogen atom, a linear or branched alkyl radical, an alkenyl radical, a mono- or polyhydroxyalkyl radical, an aryl or aralkyl radical, optionally substituted, a sugar residue or an amino acid or peptide residue,

R¹² represents a lower alkyl radical,

R′ and R″, which may be identical or different, represent a hydrogen atom, a lower alkyl radical, a mono- or polyhydroxyalkyl radical, an optionally substituted aryl radical or an amino acid, peptide or sugar residue, or alternatively, taken together, form a heterocycle,

and the optical and geometrical isomers of the said compounds of formula (I), as well as their salts.

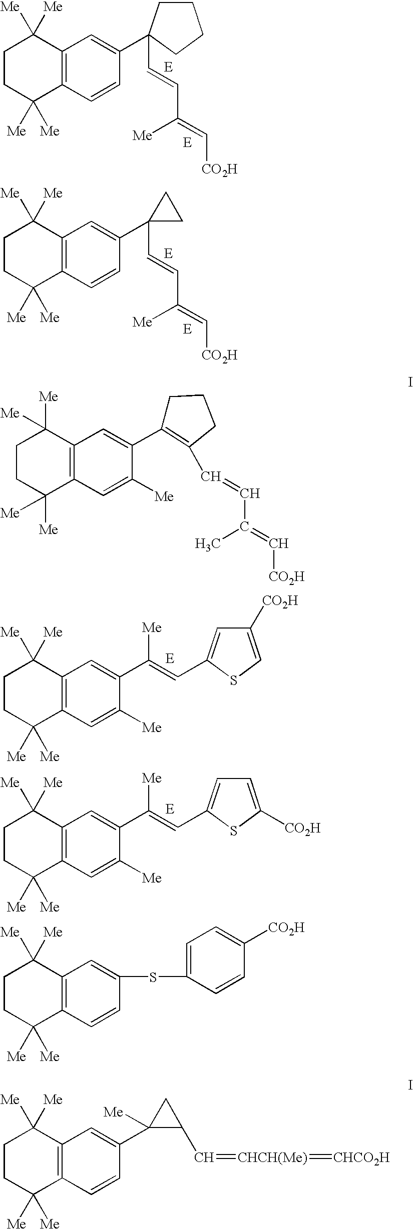

### Treatment

Treating, as used herein, refers to a reduction in (alleviation of) at least one symptom of cachexia in a patient suffering from (in need of treatment for) cachexia. Treating, as used herein, also refers to preventing the onset of at least one symptom of cachexia in a subject at risk of developing cachexia (e.g., a subject suffering from one or more of the diseases, disorders or conditions named above). Treating, as used herein, further refers to inhibiting the progression of at least one symptom of cachexia in a subject. Preferably, as with any multisymptom disorder, a reduction in or inhibition or prevention of more than one symptom is desired. The symptoms of cachexia can include loss of appetite, loss of body weight, elevation of resting energy expenditures, glucose intolerance, insulin resistance, increased fat oxidation rates, increased whole body protein turnover, decreased quality of life (e.g., decreased mobility, energy and/or stamina) and decreased life span. As such, treating of cachexia can include prevention or inhibition of appetite loss or return of appetite, prevention or inhibition of loss of body weight or an increase in body weight (e.g., as a result of preservation or restoration of lean body mass and the energy store of fat and glycogen), improvement in the patients quality of life and increased life span.

Quality of Life can be assessed by objective measurements which include nutritional and metabolic endpoints, physical function (muscle strength) and endurance (exercise tolerance). Quality of Life can also be evaluated by completing patient and caregiver questionnaires, which include standard forms such as the functional living index-cancer (FLIC), functional assessment of cancer therapy index (FACT) and the European Organization for Research and Treatment of Cancer (RORTC). The questionnaires are designed to give information regarding the effect of the drug product from a patient's and caregiver's perspective.

For the prevention or treatment of cachexia (e.g., cachexia resulting from a cancerous condition or other malignancies) it is likely that a compound of the invention is to be administered systemically. Suitable routes of administration include, but are not limited to, orally, intraperitoneally, subcutaneously, intramuscularly, intradermally, transdermally, rectally, sublingually, intravenously, buccally or via inhalation. For intravenous or intraperitoneal administration, the compound will be prepared as a solution or suspension capable of being administered by injection. In certain cases, it may be useful to formulate these compounds in suppository form or as extended release formulation for deposit under the skin or intramuscular injection. Oral admininistration of a compound in accordance with the present invention is presently preferred.

Forms suitable for oral administration include powders, pills, tablets, troches, capsules, elixirs, suspensions, syrups, wafers, chewing gum or the like prepared by art recognized procedures. The amount of active compound in such therapeutically useful compositions or preparations is such that a suitable dosage will be obtained.

The pharmaceutical compositions of the invention preferably contain a pharmaceutically acceptable carrier or diluent suitable for rendering the compound or mixture administrable orally, parenterally, intravenously, intradermally, intramuscularly or subcutaneously, rectally, via inhalation or via buccal administration, or transdermally. The active ingredients may be admixed or compounded with a conventional, pharmaceutically acceptable carrier or diluent. It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 18th ed. (1990), the disclosure of which is incorporated herein by reference.

The formulations of the present invention for use in a subject comprise the agent, together with one or more acceptable carriers or diluents therefor and optionally other therapeutic ingredients. The carriers or diluents must be “acceptable” in the sense of being compatible with the other ingredients of the formulation and not deleterious to the recipient thereof. The formulations can conveniently be presented in unit dosage form and can be prepared by any of the methods well known in the art of pharmacy. All methods include the step of bringing into association the agent with the carrier or diluent which constitutes one or more accessory ingredients. In general, the formulations are prepared by uniformly and intimately bringing into association the agent with the carriers and then, if necessary, dividing the product into unit dosages thereof.

Formulations suitable for parenteral administration conveniently comprise sterile aqueous preparations of the agents that are preferably isotonic with the blood of the recipient. Suitable carrier solutions include phosphate buffered saline, saline, water, lactated ringers or dextrose (5% in water). Such formulations can be conveniently prepared by admixing the agent with water to produce a solution or suspension, which is filled into a sterile container and sealed against bacterial contamination. Preferably, sterile materials are used under aseptic manufacturing conditions to avoid the need for terminal sterilization.

Such formulations can optionally contain one or more additional ingredients, which can include preservatives such as methyl hydroxybenzoate, chlorocresol, metacresol, phenol and benzalkonium chloride. Such materials are of special value when the formulations are presented in multidose containers.

Buffers can also be included to provide a suitable pH value for the formulation. Suitable buffer materials include sodium phosphate and acetate. Sodium chloride or glycerin can be used to render a formulation isotonic with the blood.

If desired, a formulation can be filled into containers under an inert atmosphere such as nitrogen and can be conveniently presented in unit dose or multi-dose form, for example, in a sealed ampoule.

Those skilled in the art will be aware that the amounts of the various components of the compositions of the invention to be administered in accordance with the method of the invention to a subject will depend upon those factors noted above.

The compositions of the invention when given orally or via buccal administration can be formulated as syrups, tablets, capsules and lozenges. A syrup formulation will generally consist of a suspension or solution of the compound or salt in a liquid carrier, for example, ethanol, glycerine or water, with a flavoring or coloring agent. Where the composition is in the form of a tablet, one or more pharmaceutical carriers routinely used for preparing solid formulations can be employed. Examples of such carriers include magnesium stearate, starch, lactose and sucrose. Where the composition is in the form of a capsule, the use of routine encapsulation is generally suitable, for example, using the aforementioned carriers in a hard gelatin capsule shell. Where the composition is in the form of a soft gelatin shell capsule, pharmaceutical carriers routinely used for preparing dispersions or suspensions can be considered, for example, aqueous gums, celluloses, silicates or oils, and are incorporated in a soft gelatin capsule shell.

A typical suppository formulation includes the conjugate or a pharmaceutically acceptable salt thereof which is active when administered in this way, with a binding and/or lubricating agent, for example, polymeric glycols, gelatins, cocoa-butter or other low melting vegetable waxes or fats.

Typical transdermal formulations include a conventional aqueous or non-aqueous vehicle, for example, a cream, ointment, lotion or paste or are in the form of a medicated plastic, patch or membrane.

Typical compositions for inhalation are in the form of a solution, suspension or emulsion that can be administered in the form of an aerosol using a conventional propellant such as dichlorodifluoromethane or trichlorofluoromethane.

A “subject” is typically a human, but can also be an animal in need of treatment, e.g., companion animals (e.g., dogs, cats, and the like), farm animals (e.g., cows, pigs, horses, sheep, goats and the like) and laboratory animals (e.g., rats, mice, guinea pigs and the like).

The therapeutically effective amount of a compound of the invention depends, in each case, upon several factors, e.g., the health, age, gender, size and condition of the subject to be treated, the intended mode of administration, and the capacity of the subject to incorporate the intended dosage form, among others. A therapeutically effective amount of an active agent is an amount sufficient to have the desired effect for the condition being treated. Desired treatment effects are discussed in detail above. A useful therapeutic or prophylactic concentration may vary with the severity of the condition being treated and the patient's susceptibility to treatment. Accordingly, no single concentration will be uniformly useful, but will require modification depending on the particularities of the disease being treated. Such concentrations can be arrived at through routine experimentation.

A suitable dose for mammals (e.g., humans or mammals other than humans) can range from about 0.01 to about 100 mg per kg of body weight per day, such as from about 0.1 to about 75 mg per kg of body weight per day, for example, from about 1 to about 50 mg per kg of body weight per day. More preferably, the daily dose can be from about 2 to about 25 mg per kg body weight of the mammal. In a preferred embodiment, the subject is a human and a suitable dose is about 10 to about 4000 mg per day per subject, such as about 20 to about 2000 mg per day per subject, for example, about 50 to about 1000 mg per day per subject, assuming an average human of about 70 kg. More preferably, a suitable amount is in the range from about 100 to about 500 mg per day per subject.

The method of the invention can further comprise administering an additional therapeutic agent. Preferably, the additional therapeutic agent does not diminish the effects of the primary agent(s) and/or potentiates the effect of the primary agent(s).

In one embodiment, the additional therapeutic agent can be one that is useful for treating cachexia. For example, the additional therapeutic agent can be an anticachetic agent that has a primary mechanism of action which is different from the RXR agonists described herein. Suitable anticachetic agents include, but are not limited to, progesterone derivatives (e.g., megestrol acetate and medroxyprogesterone acetate), growth hormone (e.g,. Serostim®), growth hormone secretagogues (e.g., ghrelin, GHRP-1, GHRP-2, GHRP-6, NN703, Ipamorelin, Campromorelin, MK-677 and those described in U.S. Pat. Nos. 6,303,620, 6,576,648, 5,977,178, 6,566,337, 6,083,908, 6,274,584 and published International Application No. WO 00/01726), cannabinoids (e.g., dronabinol), anabolic steroids (e.g., oxandrolone), corticosteroids (e.g., dexamethasone), monoclonal antibodies (e.g., entanercept (ENBREL® and REMICADE®)), β-Adrenergic blockers, NSAIDS, anticytokines (e.g., β-2 agonist such as clenbuterol, omega-3 fatty acids, melatonin and thalidomide), metoclopramide, insulin-like growth factor-1 (see WO 96/37216), tumor necrosis factor converting enzyme inhibitors, matrix metalloproteinase inhibitors (see WO 03/090777), appetite stimulants, melanocortin receptors, serotonin receptor inhibitors and hydrazine sulfate.

In another embodiment, the additional therapeutic agent can reduce side effects associated with the administration of the RXR agonist. For example, the additional therapeutic agent can be an antihyperlipidemic agent. Suitable antihyperlipidemic agents include, but are not limited to, bile acid sequestrants (e.g., WELCHOL®, Cholestryramine, Colestipol and Polidexide), Fibrates (e.g., Beclobrate, Bezafibrate, Binifibrate, Ciprofibrate, Clinofibrate, Clofibrate, Clofibric Acid, Etofibrate, Fenofibrate, Genfibrozil, Nicofibrate, Pirifibrate, Ronifibrate, Simfibrate and Theofibrate), HMG CoA Reductase Inhibitors (e.g., Atorvastatin, Fluvastatin, Lovastatin, Provastatin and Simvastatin), Nicotinic acid and derivatives (e.g., Acipimox, Aluminum Nicotinate, Niceritrol, Nicoclonate, Nicomol and Oxiniacic Acid), Thyroid Hormone/Analogs (e.g., Etiroxate, Thyropropic Acid and Thyroxine), and others agents such as, Acitran, Azacosterol, Benfluorex, β-Benzalbutyramide, Carnitine, Chondroitin Sulfate, Clomestrone, Detaxtran, Dextran Sulfate Sodium, 5,8,11,14,17-Eicosapentaenoic Acid, Eritadenine, Furazabol, Meflutol, Melinamide, Mytatrienediol, Ornithine, γ-Oryzanol, Pantethine, Pentaerythritol Tetraacetate, α-Phenylbutyramide, Pirozadil, Probucol, β-Sitosterol, Sultosilic Acid (Piperazine Salt), Tiadenol, Cholesterol Absorption Inhibitors (Zetia or ezetimibe) Triparanol and Xenbucin.

The term alkyl refers to and covers any and all groups which are known as normal alkyl, branched-chain alkyl and cycloalkyl. The term alkenyl refers to and covers normal alkenyl, branch chain alkenyl and cycloalkenyl groups having one or more sites of unsaturation. Similarly, the term alkynyl refers to and covers normal alkynyl, and branch chain alkynyl groups having one or more triple bonds.

Lower alkyl means the above-defined broad definition of alkyl groups having 1 to 6 carbons in case of normal lower alkyl, and as applicable 3 to 6 carbons for lower branch chained and cycloalkyl groups. Lower alkenyl is defined similarly having 2 to 6 carbons for normal lower alkenyl groups, and 3 to 6 carbons for branch chained and cyclo-lower alkenyl groups. Lower alkynyl is also defined similarly, having 2 to 6 carbons for normal lower alkynyl groups, and 4 to 6 carbons for branch chained lower alkynyl groups.

The term “ester” as used here refers to and covers any compound falling within the definition of that term as classically used in organic chemistry. It includes organic and inorganic esters.

Unless stated otherwise in this application, preferred esters are derived from the saturated aliphatic alcohols or acids of ten or fewer carbon atoms or the cyclic or saturated aliphatic cyclic alcohols and acids of 5 to 10 carbon atoms. Particularly preferred aliphatic esters are those derived from lower alkyl acids and alcohols. Also preferred are the phenyl or lower alkyl phenyl esters.

Amide has the meaning classically accorded that term in organic chemistry. In this instance it includes the unsubstituted amides and all aliphatic and aromatic mono- and di-substituted amides. Unless stated otherwise in this application, preferred amides are the mono- and di-substituted amides derived from the saturated aliphatic radicals of ten or fewer carbon atoms or the cyclic or saturated aliphatic-cyclic radicals of 5 to 10 carbon atoms. Particularly preferred amides are those derived from substituted and unsubstituted lower alkyl amines. Also preferred are mono- and disubstituted amides derived from the substituted and unsubstituted phenyl or lower alkylphenyl amines. Unsubstituted amides are also preferred.

Acetals and ketals include the radicals of the formula —CK where K is (—OR)₂. Here, R is lower alkyl. Also, K may be —OR₇O— where R₇ is lower alkyl of 2-5 carbon atoms, straight chain or branched.

A pharmaceutically acceptable salt may be prepared for any compounds in this invention having a functionality capable of forming such salt, for example an acid functionality. A pharmaceutically acceptable salt is any salt which retains the activity of the parent compound and does not impart any deleterious or untoward effect on the subject to which it is administered and in the context in which it is administered. Pharmaceutically acceptable salts may be derived from organic or inorganic bases. The salt may be a mono or polyvalent ion. Of particular interest are the inorganic ions, sodium, potassium, calcium, and magnesium. Organic salts may by be made with amines, particularly ammonium salts such as mono-, di- and trialkyl amines or ethanol amines. Salts may also be formed with caffeine, tromethamine and similar molecules. Where there is a nitrogen sufficiently basic as to be capable of forming acid addition salts, such may be formed with any inorganic or organic acids or alkylating agent such as methyl iodide. Preferred salts are those formed with inorganic acids such as hydrochloric acid, sulfuric acid or phosphoric acid. Any of a number of simple organic acids such as mono-, di- or tri-acid may also be used.

Certain compounds of the present invention have trans and cis (E and Z) isomers. In addition, the compounds of the present invention may contain one or more chiral centers and therefore may exist in enantiomeric and diastereomeric forms. The scope of the present invention is intended to cover all such isomers per se, as well as mixtures of cis and-trans isomers, mixtures of diastereomers and racemic mixtures of enantiomers (optical isomers) as well. In the present application when no specific mention is made of the configuration (cis, trans or R or S) of a compound (or of an asymmetric carbon) then a mixture of such isomers, or either one of the isomers is intended. In a similar vein, when in the chemical structural formulas of this application a straight line representing a valence bond is drawn to an asymmetric carbon, then isomers of both R and S configuration, as well as their mixtures are intended. A straight horizontal single line or a wavy single line drawn to a carbon with a double bond denotes either cis or trans or both orientations of the substituent on the double bond. Specific orientation of substituents relative to a double bond is indicated in the name of the respective compound, and/or by specifically showing in the structural formula the orientation of the substituents relative to the double bond.

### Exemplification

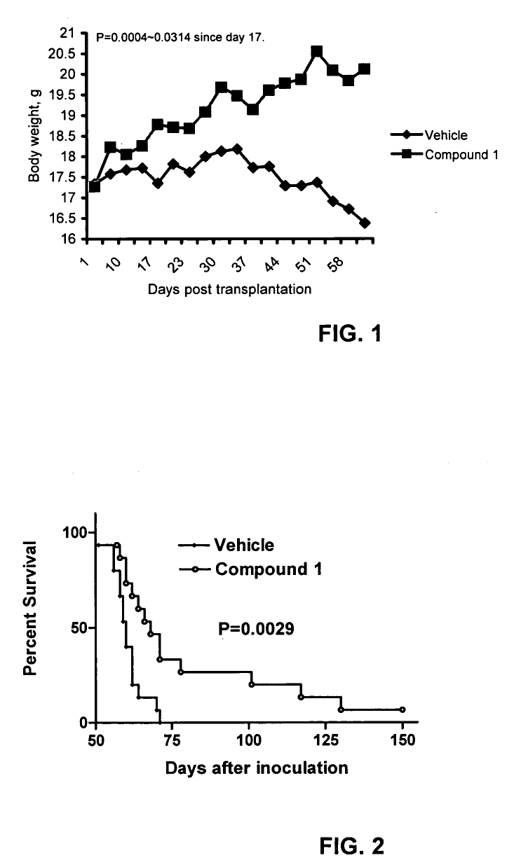

FIGS. 1-5 comprise charts or graphs disclosing the results of tests obtained with experimental animals that have been inoculated with a xenograft of non-small cell lung cancer cells H292 or with small cell lung cancer cells H446, and which were then orally administered the RXR agonist Compound 1 referred to above.

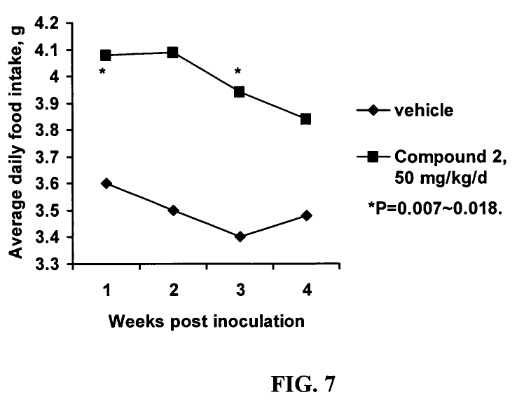

FIGS. 6 and 7 disclose results of tests obtained with experimental animals that have been inoculated with a xenograft of non-small cell lung cancer cells H292 and which were then orally administered the RXR agonist Compound 2 referred to above.

Specifically, in the experiment shown in FIG. 1 nude mice were subcutaneously transplanted with non-small cell lung cancer cells H292. A group of the animals was given a daily oral dose of 10 mg per kilogram body weight of Compound 1 in a suitable pharmaceutically acceptable vehicle. A group of the control animals was given the vehicle only. The graph shows the body weight of the animals in grams. It can be seen that the animals treated with Compound 1 have significantly greater body weights than the animals which received the vehicle only.

FIG. 2 shows the percentage of survival of nude mice from a similar experiment as the one described in connection with FIG. 1, and demonstrates significantly better survival rate for the animals that received Compound 1 in a daily oral dose of 10 mg per kg body weight of the animal.

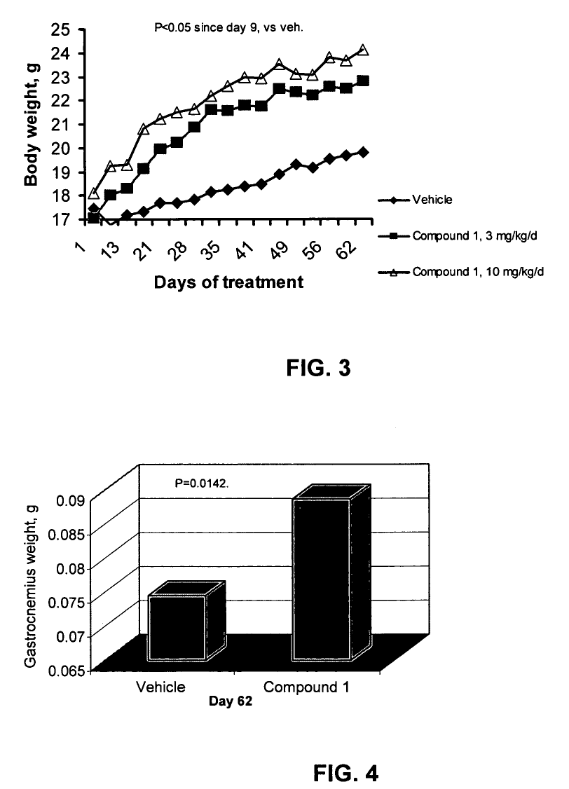

In the experiment shown in FIG. 3, SCID mice were subcutaneously transplanted with small cell lung cancer cells H446. A first group of the animals was given a daily oral dose of 3 mg per kilogram body weight of Compound 1 in a suitable pharmaceutically acceptable vehicle, and a second group was given a daily oral dose of 10 mg per kilogram body weight in the same vehicle. A group of the control animals was given the vehicle only. The graph shows the body weight of the animals in grams. It can be seen that the animals treated with Compound 1 have significantly greater body weights than the animals that received the vehicle only.

In the experiment shown in FIG. 4 the right gastrocnemius muscle of the control animals and of the animals treated with Compound 1 in a daily dose of 10 mg/kg, as described in connection with FIG. 1, was weighed after the animals had been sacrificed. It can be seen that treatment in accordance with the invention prevents muscle wasting.

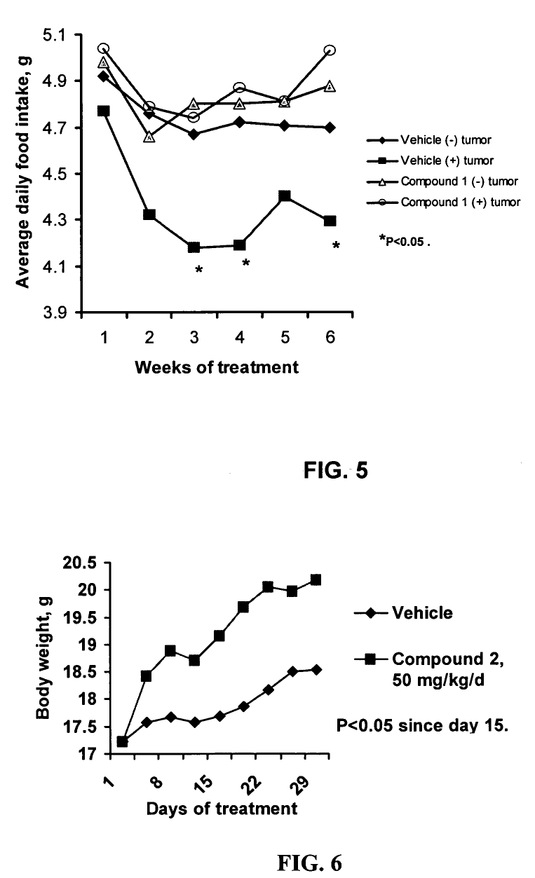

In the experiment shown in FIG. 5, the food intake of nude mice with and without H292 xenografts was evaluated. Mice with H292 xenografts had reduced appetite compared with normal control. Mice treated with Compound 1 in a daily dose of 10 mg/kg body weight had equal amount of food intake as normal mice. Therefore, adminstration of Compound 1 reverses poor appetite in cachectic animals.

In the experiment shown in FIG. 6, nude mice were subcutaneously transplanted with non-small cell lung cancer cells H292. A group of the animals was given a daily oral dose of 50 mg per kilogram body weight of Compound 2 in a suitable pharmaceutically acceptable vehicle. A group of the control animals was given the vehicle only. The graph shows the body weight of the animals in grams. It can be seen that the animals treated with Compound 2 have significantly greater body weights than the animals which received the vehicle only.

In the experiment shown in FIG. 7, the food intake of nude mice bearing H292 xenografts was evaluated. Mice treated with Compound 2 in a daily dose of 50 mg/kg body weight had significantly larger food intake than the tumor bearing mice which received only vehicle. Therefore, adminstration of Compound 2 significantly increases the appetite of tumor bearing animals.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating cachexia in a subject in need thereof, the method comprising administering to said subject a therapeutically effective amount of a compound represented by Structural Formula (I): 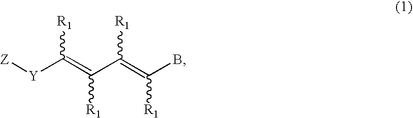
wherein:
Z is represented by Structural Formula (II) or Structural Formula (III) 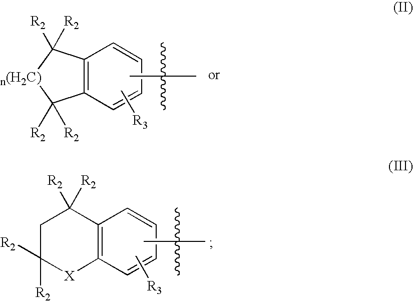
Y is cycloalkyl of 3 to 8 carbons or cycloalkenyl of 5 to 8 carbons optionally substituted with one or two R₄ groups, or Y is selected from phenyl, pyridyl, thienyl, furyl, pyrrolyl, pyridazinyl, pyrimidinyl, pyrazinyl, thiazolyl, oxazolyl, and imidazolyl, said groups being optionally substituted with one or two R₄ groups, and wherein Y is substituted by the Z and —CR₁═CR₁—CR₁═CR₁— groups on adjacent carbons;
X is S, O, or NR₅;
n is 1 or 2;
R₁ and R₂ independently are —H, lower alkyl or fluoroalkyl;
R₃ is hydrogen, lower alkyl, alkylamino, dialkylamino, cyano, —Cl or —Br;
R₄ is lower alkyl, fluoroalkyl or halogen;
R₅ is H or lower alkyl;
B is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂ or —CR₇OR₁₃O;
R₇ is an alkyl, cycloalkyl or alkenyl group containing 1 to 5 carbons;
R₈ is an alkyl group of 1 to 10 carbons, a cycloalkyl group of 5 to 10 carbons or trimethylsilylalkyl, where the alkyl group has 1 to 10 carbons, or R₈ is phenyl or lower alkylphenyl;
R₉ and R₁₀ independently are hydrogen, an alkyl group of 1 to 10 carbons, or a cycloalkyl group of 5 to 10 carbons, or phenyl or lower alkylphenyl;
R₁₁ is lower alkyl, phenyl or lower alkylphenyl;
R₁₂ is lower alkyl; and
R₁₃ is divalent alkyl radical of 2 to 5 carbons.

2. The method of claim 1, wherein Y is cyclopropyl, phenyl, pyridyl, thienyl or furyl.

3. The method of claim 2, wherein Y is cyclopropyl or phenyl.

4. The method of claim 3, wherein Y is

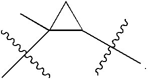

5. The method of claim 1, wherein R₁ is H or methyl.

6. The method of claim 1, wherein B is —COOH or a pharmaceutically acceptable salt thereof, —COOR₈ or —CONR₉R₁₀.

7. The method of claim 1, wherein Z is represented by Structural Formula (II) and n is 2.

8. The method of claim 1, wherein Z is represented by Structural Formula (III) and X is S or O.

9. The method of claim 1, wherein the cachexia is associated with cancer.

10. The method of claim 9, wherein the cancer is lung cancer, colorectal cancer, pancreatic cancer, gastrointestinal cancer, liver cancer, biliary cancer, breast cancer, esophageal cancer or leukemia.

11. The method of claim 1, wherein the cachexia is associated with one or more diseases, disorders or conditions selected from the group consisting of cancer, AIDS, liver cirrhosis, diabetes mellitus, chronic renal failure, chronic obstructive pulmonary disease, chronic cardiac failure, immune system diseases, tuberculosis, cystic fibrosis, gastrointestinal disorders, Parkinson's disease, anorexia nervosa, dementia, major depression, an aged condition and sarcopenia.

12.-19. (canceled)

20. The method of claim 1, wherein the compound is represented by Structural Formula (IV): 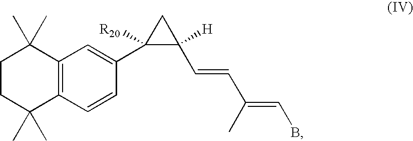
wherein:
R₂₀ is alkyl of 1 to 6 carbons;
B is —COOH, or —COOR₂₁; and
R₂₁ is alkyl of 1 to 6 carbons,
or a pharmaceutically acceptable salt of said compound.

21.-29. (canceled)

30. The method of claim 20, wherein the compound is represented by the formula: 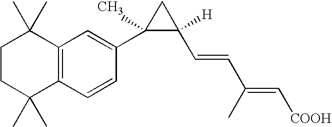
or a pharmaceutically acceptable salt of said compound.

31. The method of claim 20, wherein the compound is represented by the formula: 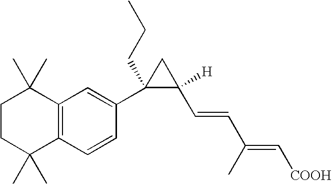
or a pharmaceutically acceptable salt of said compound.

32. The method of claim 1 the compound is represented by Structural Formula (V): 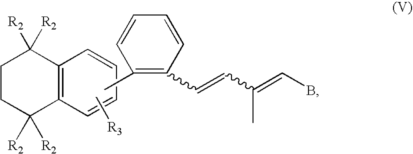
wherein:
R₂ is hydrogen or lower alkyl; and
R₃ is hydrogen or lower alkyl.

33.-41. (canceled)

42. The method of claim 1, wherein the compound is represented by Structural Formula (VI): 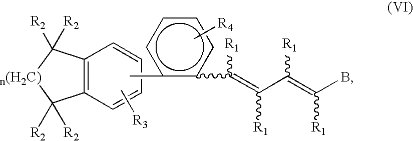
wherein:
R₃ is hydrogen, lower alkyl, —Cl or —Br; and
R₄ is H, lower alkyl, trifluoromethyl or halogen.

43.-51. (canceled)

52. The method of claim 1, wherein the compound is represented by Structural Formula (VII): 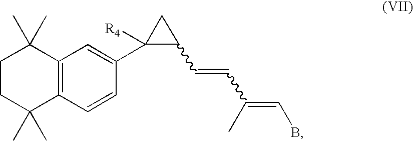
wherein:
R₄ is lower alkyl of 1 to 6 carbons;
B is —COOH or —COOR₈; and
R₈ is lower alkyl of 1 to 6 carbons; and
the configuration about the cyclopropane ring is cis, and the configuration about the double bonds in the pentadienoic acid or ester chain attached to the cyclopropane ring is trans in each of said double bonds, or a pharmaceutically acceptable salt of said compound.

53.-61. (canceled)

62. The method of claim 1,
wherein:
Y is selected from pyridyl, pyrrolyl, pyridazinyl, pyrimidinyl, pyrazinyl, thiazolyl, oxazolyl, and imidazolyl, said groups being optionally substituted with one or two R₄ groups, and wherein Y is substituted by the Z and —CR₁═CR₁—CR₁═CR₁— groups on adjacent carbons; and
X is NR₅.

63.-71. (canceled)

72. The method of claim 1,
wherein:
Z is represented by Structural Formula (III) 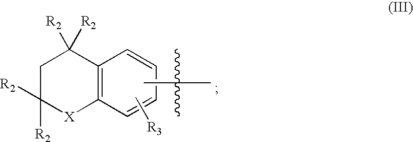
Y is thienyl or furyl, said thienyl or furyl groups being optionally substituted with one or two R₄ groups, and wherein Y is substituted by the Z and —CR₁═CR₁—CR₁═CR₁— groups on adjacent carbons; and
X is NR₅.

73.-91. (canceled)

92. The method of claim 1, wherein the compound is represented by Structural Formula (VIII): 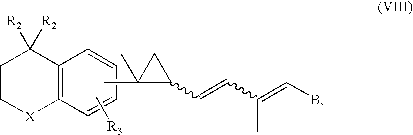
wherein:
X is S or O;
R₂ is hydrogen or lower alkyl; and
R₃ is hydrogen or lower alkyl.

93.-101. (canceled)

102. The method of claim 1,
wherein:
Z is represented by Structural Formula (II) 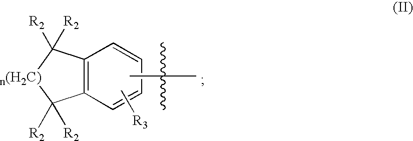
Y is selected from thienyl or furyl, said groups being optionally substituted with one or two R₄ groups, and wherein Y is substituted by the Z and —CR₁═CR₁—CR₁═CR₁— groups on adjacent carbons.

103.-111. (canceled)

112. The method of claim 1,
wherein:
Z is represented by Structural Formula (III) 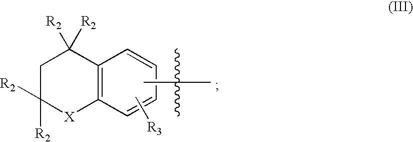
Y is cycloalkyl of 3 to 8 carbons or cycloalkenyl of 5 to 8 carbons optionally substituted with one or two R₄ groups, or Y is phenyl, said groups being optionally substituted with one or two R₄ groups, and wherein Y is substituted by the Z and —CR₁═CR₁—CR₁═CR₁— groups on adjacent carbons; and
X is NR₅.

113.-121. (canceled)

122. The method of claim 1,
wherein:
Z is represented by Structural Formula (III) 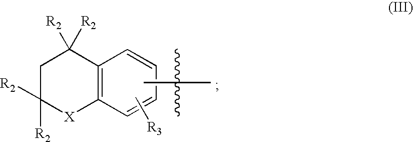
Y is cyclopropyl, said Y group being optionally substituted with one or two R₄ groups, and wherein Y is substituted by the Z and —CR₁═CR₁—CR₁═CR₁— groups on adjacent carbons;
X is NR₅; and
R₇ is an alkyl of 1 to 5 carbons, cycloalkyl of 3 to 5 carbons or alkenyl group containing 2 to 5 carbons.

123.-131. (canceled)

132. The method of claim 1, wherein the compound is represented by Structural Formula (VIII): 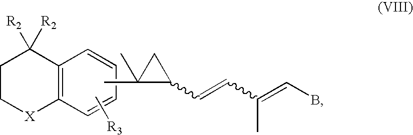
wherein:
X is NR₅;
R₂ is hydrogen or lower alkyl;
R₃ is hydrogen or lower alkyl; and
R₇ is an alkyl of 1 to 5 carbons, cycloalkyl of 3 to 5 carbons or alkenyl group containing 2 to 5 carbons.

133.-141. (canceled)

142. The method of claim 1, wherein the compound is represented by Structural Formula (IX), (X) or (XI): 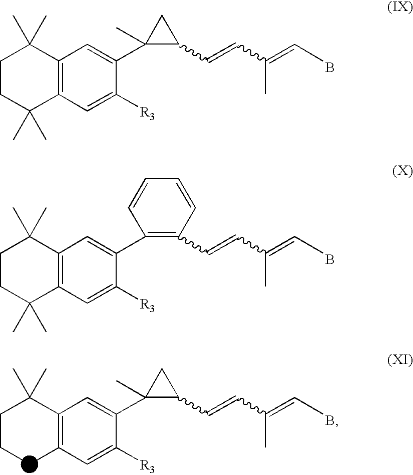
wherein:
B is —COOH or —COOR₈;
R₃ is hydrogen, lower alkyl, Cl or Br;
X is S or O.

143.-151. (canceled)

152. The method of claim 142, wherein the compound is represented by Structural Formula (IX), R₃ is H or methyl and B is —COOH or —COOCH₂CH₃.

153. The method of claim 142, wherein the compound is represented by Structural Formula (X), R₃ is H and B is —COOH or —COOCH₂CH₃.

154. The method of claim 142, wherein the compound is represented by Structural Formula (XI), R₃ is H, B is —COOH or —COOCH₂CH₃ and X is O or S.

155. The method of claim 1, wherein the compound is represented by Structural Formula (XII): 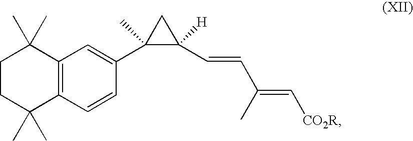
wherein R is hydrogen or lower alkyl of 1 to 6 carbons, or a pharmaceutically acceptable salt thereof.

156.-164. (canceled)

165. A method of treating cachexia in a subject in need thereof, the method comprising administering to said subject a therapeutically effective amount of a compound represented by Structural Formula (XIII), (XIV) or (XV): 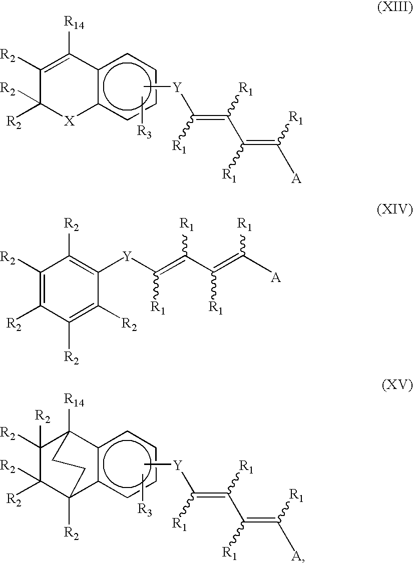
wherein:
X is O, S, or (CR₁R₁)ₙ;
n is 0, 1 or 2;
Y is a bivalent radical having Structural Formula (XVI) or Structural Formula (XVII) where p is an integer from 1 to 4: 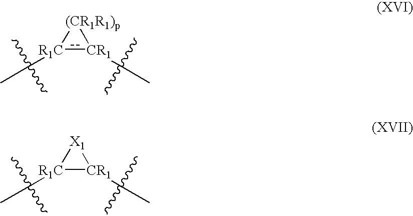
or Y is a bivalent aryl or 5 or 6 membered heteroaryl radical having 1 to 3 heteroatoms selected from N, S and O, said aryl or heteroaryl groups being unsubstituted, or substituted with 1 to 3 C₁₋₆ alkyl or with 1 to 3 C₁₋₆ fluoroalkyl groups;
X is O, S or NH;
R₁ is independently H, lower alkyl of 1 to 6 carbons, or lower fluoroalkyl of 1 to 6 carbons;
R₂ is independently —H, lower alkyl of 1 to 6 carbons, —OR₁, 1-adamantyl, or lower fluoroalkyl of 1 to 6 carbons, or the two R₂ groups jointly represent an oxo group;
R₃ is hydrogen, lower alkyl of 1 to 6 carbons, —OR₁, fluoro substituted lower alkyl of 1 to 6 carbons or halogen, —NO₂, —NH₂, —NHCO(C₁-C₆)alkyl, or —NHCO(C₁-C₆)alkenyl;
A is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CH(OR₁₃O), —COR₇, —CR₇(OR₁₂)₂, —CR₇(OR₁₃P), or —Si(C₁₋₆ alkyl)₃;
R₇ is an alkyl, cycloalkyl or alkenyl group containing 1 to 5 carbons;
R₈ is an alkyl group of 1 to 10 carbons or (trimethylsilyl)alkyl where the alkyl group has 1 to 10 carbons, or a cycloalkyl group of 5 to 10 carbons, or R₈ is phenyl or lower alkylphenyl;
R₉ and R₁₀ independently are hydrogen, an alkyl group of 1 to 10 carbons, or a cycloalkyl group of 5-10 carbons, or phenyl, hydroxyphenyl or lower alkylphenyl;
R₁₁ is lower alkyl, phenyl or lower alkylphenyl;
R₁₂ is lower alkyl;
R₁₃ is divalent alkyl radical of 2-5 carbons; and
R₁₄ is alkyl of 1 to 10 carbons, fluoro-substituted alkyl of 1 to 10 carbons, alkenyl of 2 to 10 carbons and having 1 to 3 double bonds, alkynyl having 2 to 10 carbons and 1 to 3 triple bonds, carbocyclic aryl selected from the group consisting of phenyl, C₁-C₁₀-alkylphenyl, naphthyl, C₁-C₁₀-alkylnaphthyl, phenyl-C₁-C₁₀ alkyl, naphthyl-C₁-C₁₀ alkyl, C₁-C₁₀-alkenylphenyl having 1 to 3 double bonds, C₁-C₁₀-alkynylphenyl having 1 to 3 triple bonds, phenyl-C₁-C₁₀ alkenyl having 1 to 3 double bonds, phenyl-C₁-C₁₀ alkynyl having 1 to 3 triple bonds, hydroxy alkyl of 1 to 10 carbons, hydroxyalkenyl having 2 to 10 carbons and 1 to 3 double bonds, hydroxyalkynyl having 2 to 10 carbons and 1 to 3 triple bonds, acyloxyalkyl of 1 to 10 carbons, acyloxyalkenyl having 2 to 10 carbons and 1 to 3 double bonds, or acyloxyalkynyl of 2 to 10 carbons and 1 to 3 triple bonds where the acyl group is represented by COR₈, or R₁₄ is a 5 or 6 membered heteroaryl group having 1 to 3 heteroatoms, said heteroatoms being selected from a group consisting of O, S, and N, said heteroaryl group being unsubstituted or substituted with a C₁ to C₁₀ alkyl group, with a C₁ to C₁₀ fluoroalkyl group, or with halogen, and the dashed line in Structural Formula (XVI) represents a bond or absence of a bond.

166.-174. (canceled)

175. A method of treating cachexia in a subject in need thereof, the method comprising administering to said subject a therapeutically effective amount of a compound represented by Structural Formula (XVIII): 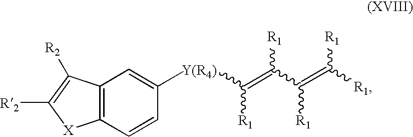
wherein:
X is O, NR′ or S;
R′ is alkyl of 1 to 6 carbons;
Y is a bivalent cyclopropyl radical optionally substituted with one or two R₄ groups, or Y is a bivalent aryl or 5 or 6 membered heteroaryl radical having 1 to 3 heteroatoms selected from N, S and O, said aryl or heteroaryl groups optionally substituted with 1 to 4 R₄ groups;
R₁ is independently H, alkyl of 1 to 6 carbons, or fluoroalkyl of 1 to 6 carbons;
R₂ is alkyl of 1 to 8 carbons, or fluoroalkyl of 1 to 8 carbons;
R′₂ is alkyl of 1 to 8 carbons, or fluoroalkyl of 1 to 8 carbons;
R₃ is hydrogen, alkyl of 1 to 6 carbons, fluoro substituted alkyl of 1 to 6 carbons, halogen, alkoxy of 1 to 8 carbons, or alkylthio of 1 to 6 carbons, —NO₂, —NH₂, —NHCO(C₁-C₆)alkyl, —NHCO(C₁-C₆)alkenyl, —NR₁H or —N(R₁)₂, benzyloxy or C₁-C₆ alkyl-substituted benzyloxy;
R₄ is —H or alkyl of 1 to 6 carbons, or fluoro substituted alkyl of 1 to 6 carbons;
m is an integer having the values of 0 to 3, and
B is —COOH or a pharmaceutically acceptable salt thereof, —COOR₈, —COOCH₂COR₇, —CONR₉R₁₀, —CH₂OH, —CH₂OR₁₁, —CH₂OCOR₁₁, —CHO, —CH(OR₁₂)₂, —CH(OR₁₃O), —COR₇, —CR₇(OR₁₂)₂, —CR₇(OR₁₃O),
R₇ is an alkyl, cycloalkyl or alkenyl group containing 1 to 5 carbons;
R₈ is an alkyl group of 1 to 10 carbons or (trimethylsilyl)alkyl where the alkyl group has 1 to 10 carbons, or a group of 5 to 10 phenyl or lower alkylphenyl;
R₉ and R₁₀ independently are hydrogen, an alkyl group of 1 to 10 carbons, or a cycloalkyl group of 5-10 carbons, or phenyl, hydroxyphenyl or lower alkylphenyl;
R₁₁ is lower alkyl, phenyl or lower alkylphenyl;
R₁₂ is lower alkyl; and
R₁₃ is divalent alkyl radical of 2-5 carbons.

176.-184. (canceled)

185. A method of treating cachexia in a subject in need thereof, the method comprising administering to said subject a therapeutically effective amount of a compound represented by Structural Formula (XIX): 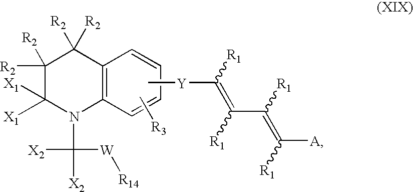
wherein:
Y is a bivalent radical having Formula (a) or Formula (b): 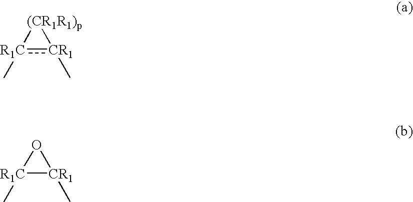
or Y is a bivalent aryl or 5 or 6 membered heteroaryl radical having 1 to 3 heteroatoms selected from N, S and O, said aryl or heteroaryl groups being unsubstituted, or substituted with 1 to 3 C₁₋₆ alkyl or with 1 to 3 C₁₋₆ fluoroalkyl groups;
p is an integer from 1 to 4;
the two X₁ groups jointly represent an oxo or thione function, or X₁ is independently selected from H or alkyl of 1 to 6 carbons;
the two X₂ groups jointly represent an oxo or a thione function, or X₂ is independently selected from H or alkyl of 1 to 6 carbons, with the proviso that one of the joint X₁ grouping or of the joint X₂ grouping represents an oxo or a thione function;
W is H, O, C(R₁)₂, phenyl, naphthyl, or 5 or 6 membered heteroaryl group having 1 to 3 heteroatoms, said heteroatoms being selected from a group consisting of O, S, and N, said phenyl, naphthyl or heteroaryl groups being unsubstituted or substituted with a C₁ to C₁₀ alkyl group, with a C₁ to C₁₀ fluoroalkyl group, or with halogen;
R₁ is independently H, lower alkyl of 1 to 6 carbons, or lower fluoroalkyl of 1 to 6 carbons;
R₂ is independently H, lower alkyl of 1 to 6 carbons, or lower fluoroalkyl of 1 to 6 carbons;
R₃ is hydrogen, lower alkyl of 1 to 6 carbons, —OR₁, fluoro substituted lower alkyl of 1 to 6 carbons or halogen, —NO₂, —NH₂, —NHCO(C₁-C₆) alkyl, or NHCO(C₁-C₆)alkenyl;
A is hydrogen, —COOH or a pharmaceutically acceptable salt thereof, —COOR₈, —CONR₉R₁₀, —CH₂OH, —CH₂OR₁₁, —CH₂OCOR₁₁, —CHO, —CH(OR₁₂)₂, —CH(OR₁₃O), —COR₇, —CR₇(OR₁₂)₂, —CR₇(OR₁₃O), or —Si(C₁₋₆ alkyl)₃;
R₇ is an alkyl, cycloalkyl or alkenyl group containing 1 to 5 carbons,
R₈ is an alkyl group of 1 to 10 carbons or (trimethylsilyl)alkyl where the alkyl group has 1 to 10 carbons, or a cycloalkyl group of 5 to 10 carbons, or R₈ is phenyl or lower alkylphenyl;
R₉ and R₁₀ independently are hydrogen, an alkyl group of 1 to 10 carbons, or a cycloalkyl group of 5-10 carbons, or phenyl, hydroxyphenyl or lower alkylphenyl;
R₁₁ is lower alkyl, phenyl or lower alkylphenyl;
R₁₂ is lower alkyl;
R₁₃ is divalent alkyl radical of 2-5 carbons;
R₁₄ is H, alkyl of 1 to 10 carbons, fluoro-substituted alkyl of 1 to 10 carbons, alkenyl of 2 to 10 carbons and having 1 to 3 double bonds, alkynyl having 2 to 10 carbons and 1 to 3 triple bonds, carbocyclic aryl selected from the group consisting of phenyl, C₁-C₁₀-alkylphenyl, naphthyl, C₁-C₁₀-alkylnaphthyl, phenyl-C₁-C₁₀ alkyl, naphthyl-C₁-C₁₀-alkyl, C₁-C₁₀-alkenylphenyl having 1 to 3 double bonds, C₁-C₁₀-alkynylphenyl having 1 to 3 triple bonds, phenyl-C₁-C₁₀ alkenyl having 1 to 3 double bonds, phenyl-C₁-C₁₀ alkynyl having 1 to 3 triple bonds, hydroxy alkyl of 1 to 10 carbons, hydroxyalkenyl having 2 to 10 carbons and 1 to 3 double bonds, hydroxyalkynyl having 2 to 10 carbons and 1 to 3 triple bonds, acyloxyalkyl of 1 to 10 carbons, acyloxyalkenyl having 2 to 10 carbons and 1 to 3 double bonds, or acyloxyalkynyl of 2 to 10 carbons and 1 to 3 triple bonds where the acyl group is represented by COR₈, or R₁₄ is a 5 or 6 membered heteroaryl group having 1 to 3 heteroatoms, said heteroatoms being selected from a group consisting of O, S, and N, said carbocyclic aryl and heteroaryl groups being unsubstituted or substituted with a C₁ to C₁₀ alkyl group, with a C₁ to C₁₀ fluoroalkyl group, or with halogen;
and the dashed line in Formula (a) represents a bond or absence of a bond, provided that when the dashed line represents a bond then there are no R₁ substituents on the carbons connected by said bond.

186.-194. (canceled)

195. A method of treating cachexia in a subject in need thereof, the method comprising administering to said subject a therapeutically effective amount of a compound represented by Structural Formula (XX): 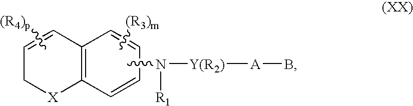
wherein:
X is O, S, or C(R)₂;
R is H or alkyl of 1 to 6 carbons;
R₁ is H, alkyl of 1 to 10 carbons, alkenyl of 2 to 6 carbons, phenyl-C₁-C₆ alkyl, or C₁-C₆-alkylphenyl;
R₂ is H, alkyl of 1 to 6 carbons, —F, —Cl, —Br, —I, —CF₃, fluoro substituted alkyl of 1 to 6 carbons, alkoxy of 1 to 6 carbons, or alkylthio of 1 to 6 carbons;
R₃ is independently alkyl of 1 to 6 carbons, —F, —Cl, —Br, —I, —CF₃, fluoro substituted alkyl of 1 to 6 carbons, OH, SH, alkoxy of 1 to 6 carbons, fluoroalkoxy of 1 to 6 carbons, alkylthio of 1 to 6 carbons, benxyloxy, C₁-C₆ alkyl substituted benzyloxy, halogen substituted benzyloxy, phenyloxy, C₁-C₆ alkyl substituted phenyloxy, or halogen substituted phenyloxy;
R₄ is independently —H, alkyl of 1 to 6 carbons, or —F;
Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups; m is an integer having the values 0 to 3;
p is an integer having the values 0 to 4;
A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;
B is hydrogen, —COOH, —COOR₈, —CONR₉R₁₀, —CH₂OH, —CH₂OR₁₁, —CH₂OCOR₁₁, —CHO, —CH(OR₁₂)₂, —CHOR₁₃O, —COR₇, —CR₇(OR₁₂)₂, —CR₇OR₁₃O, or tri-lower alkylsilyl;
R₇ is an alkyl, cycloalkyl or alkenyl group containing 1 to 5 carbons,
R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl;
R₉ and R₁₀ independently are hydrogen, an alkyl group of 1 to 10 carbons, or a cycloalkyl group of 5-10 carbons, or phenyl or lower alkylphenyl;
R₁₁ is lower alkyl, phenyl or lower alkylphenyl;
R₁₂ is lower alkyl; and
R13 is divalent alkyl radical of 2-5 carbons,
or a pharmaceutically acceptable salt thereof.

196.-204. (canceled)

205. A method of treating cachexia associated with one or more diseases, disorders or conditions selected from the group consisting of cancer, AIDS, liver cirrhosis, chronic renal failure, chronic obstructive pulmonary disease, chronic cardiac failure, immune system diseases, tuberculosis, cystic fibrosis, gastrointestinal disorders, Parkinson's disease, anorexia nervosa, dementia, major depression, an aged condition and sarcopenia in a subject in need thereof, the method comprising administering to said subject a therapeutically effective amount of an RXR agonist compound.

206.-218. (canceled)

219. A method of treating cachexia associated with one or more diseases, disorders or conditions selected from the group consisting of cancer, AIDS, liver cirrhosis, chronic renal failure, chronic obstructive pulmonary disease, chronic cardiac failure, immune system diseases, tuberculosis, cystic fibrosis, gastrointestinal disorders, Parkinson's disease, anorexia nervosa, dementia, major depression, an aged condition and sarcopenia in a subject in need thereof, the method comprising administering to said subject a therapeutically effective amount of a compound represented by Structural Formula (XXI), (XXII), (XXIII), (XXIV), (XXV), (XXVI), (XXVII), (XXVIIa) or (XXVIIb): 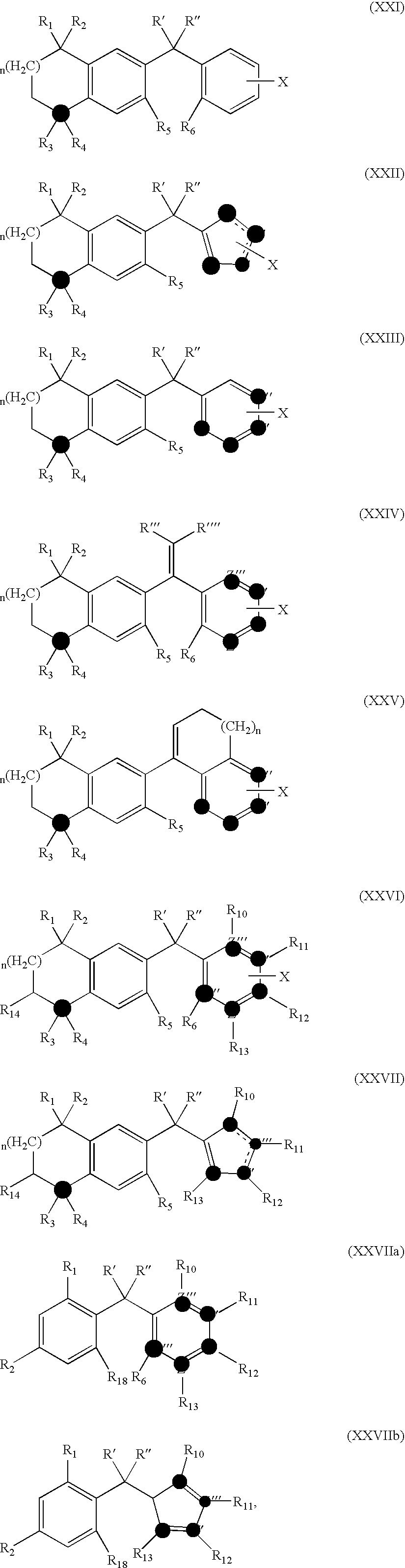
wherein:
R₁ and R₂ each independently is hydrogen or lower alkyl or acyl having 1-4 carbon atoms;
Y is C, O, S, N, CHOH, CO, SO, SO₂, or a pharmaceutically acceptable salt;
R₃ is hydrogen or lower alkyl having 1-4 carbon atoms where Y is C or N;
R₄ is hydrogen or lower alkyl having 1-4 carbon atoms when Y is C, R₄ does not exist if Y is N, or neither R₃ or R₄ exist if Y is S, O, CHOH, CO, SO, or SO₂;
R′ and R″ are hydrogen, lower alkyl or acyl having 1-4 carbon atoms, OH, alkoxy having 1-4 carbon atoms, thiol or thioether, or amino, or R′ or R″ taken together form an oxo(keto), methano, thioketo, HO—N═, NC—N═, (R₇R₈)N—N═, R₁₇O—N═, R₁₇N═, epoxy, cyclopropyl, or cycloalkyl group and wherein the epoxy, cyclopropyl, and cycloalkyl groups are optionally substituted with lower alkyl having 1-4 carbons or halogen;
R′″ and R″″ are hydrogen, halogen, lower alkyl or acyl having 1-4 carbon atoms, alkylamino, or R′″ and R″″ taken together form a cycloalkyl group having 3-10 carbons, and wherein the cycloalkyl group can be substituted with lower alkyl having 1-4 carbons or halogen;
R₅ is hydrogen, a lower alkyl having 1-4 carbons, halogen, nitro, —OR₇, —SR₇, —NR₇R₈, or —(CF)ₙCF₃, but R₅ is not hydrogen if R₆, R₁₀, R₁₁, R₁₂ and R₁₃ are all hydrogen, Z, Z′, Z″, Z′″, and Z″″ are all carbon, and R′ and R″ represent —H, —OH, C₁-C₄ alkoxy or C₁-C₄ acyloxy or R′ and R″ taken together form an oxo, methano, or hydroxyimino group;
R₆, R₁₀, R₁₁, R₁₂ and R₁₃ each independently represent hydrogen, a lower alkyl having 1-4 carbons, halogen, nitro, —OR₇, —SR₇, —NR₇R₈ or —(CF)ₙCF₃, and exist only if the Z, Z′, Z″, Z′″, or Z″″ from which R₆, R₁₀, R₁₁, R₁₂ or R₁₃ originates is C, or R₆, R₁₀, R₁₁, R₁₂ and R₁₃ each independently represent hydrogen or a lower alkyl having 1-4 carbons if the Z, Z′, Z′″, Z″, or Z″″ from which R₆, R₁₀, R₁₁, R₁₂ or R₁₃ originates is N, and where one of R₆, R₁₀, R₁₁, R₁₂ or R₁₃ is X;
R₇ represents hydrogen or a lower alkyl having 1-6 carbons;
R₈ represents hydrogen or a lower alkyl having 1-6 carbons;
R₉ represents a lower alkyl having 1-4 carbons, phenyl, aromatic alkyl, or q-hydroxyphenyl, q-bromophenyl, q-chlorophenyl, q-florophenyl, or q-iodophenyl, where q=2-4;
R₁₄ represents hydrogen, a lower alkyl having 1-4 carbons, oxo, hydroxy, acyl having 1-4 carbons, halogen, thiol, or thioketone;
R₁₇ is hydrogen, lower alkyl having 1-8 carbons, alkenyl optionally substituted with halogen, acyl, —OR₇ or —SR₇, —R₉, alkyl carboxylic acid optionally substituted with halogen, acyl, —OR₇ or —SR₇, alkenyl carboxylic acid optionally substituted with halogen, acyl, —OR₇ or —SR₇, alkyl amine optionally substituted with halogen, acyl, —OR₇ or —SR₇, or alkenyl amine optionally substituted with halogen, acryl, —OR₇ or —SR₇;
R₁₈ represents hydrogen, a lower alkyl having 1-4 carbons, halogen, nitro, —OR₇, —SR₇, —NR₇R₈, or —(CF)ₙCF₃;
X is —COOH, tetrazole, —PO₃H, —SO₃H, —CHO, —CH₂OH, —CONH₂, —COSH, —COOR₉, —COSR₉, —CONHR₉, or —COOW where W is a pharmaceutically acceptable salt, and wherein X can originate from any C or N on the ring;
Z, Z′, Z″, Z′″ and Z″″ each independently is C, S, O, N, or a pharmaceutically acceptable salt, provided that one or more of Z, Z′, Z″, Z′″ and Z″″ are not O or S if Z, Z′, Z″, Z′″ or Z″″ is attached by a double bond to one of Z, Z′, Z″, Z′″ or Z″″ or if one or more of Z, Z′, Z″, Z′″ or Z″″ is attached to one of Z, Z′, Z″, Z′″ or Z″″ that is O or S, and provided that one or more of Z, Z′, Z″, Z′″ and Z″″ are not N if one of Z, Z′, Z″, Z′″ and Z″″ is attached by a single bond to one of Z, Z′, Z″, Z′″ and Z″″ that is N;
n is 0 to 3; and
the dashed lines are optional double bonds.

220. The method of claim 219, wherein the RXR agonist compound is represented by the formula: 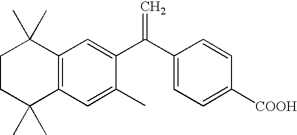
or a pharmaceutically acceptable salt of said compound.

221. The method of claim 219, wherein the cachexia is associated with cancer.

222. The method of claim 221, wherein the cancer is lung cancer, colorectal cancer, pancreatic cancer, gastrointestinal cancer, liver cancer, biliary cancer, breast cancer, esophageal cancer or leukemia.

223. The method of claim 219, wherein the cachexia is associated with one or more diseases, disorders or conditions selected from the group consisting of cancer, AIDS, liver cirrhosis, chronic renal failure, chronic obstructive pulmonary disease, chronic cardiac failure, immune system diseases, tuberculosis, cystic fibrosis, gastrointestinal disorders, Parkinson's disease, anorexia nervosa, dementia, major depression, an aged condition and sarcopenia.

224.-229. (canceled)
